(19) United States
(12) Patent Application Publication
(10) Pub. No.: US 2024/0368224 A1
(43) Pub. Date: Nov. 7, 2024

(54) BICYCLIC PEPTIDYL PAN-RAS INHIBITORS

(71) Applicant: Ohio State Innovation Foundation, Columbus, OH (US)

(72) Inventors: Dehua PEI, Columbus, OH (US); Marina Buyanova, Columbus, OH (US)

(21) Appl. No.: 18/572,885

(22) PCT Filed: Jun. 22, 2022
(86) PCT No.: PCT/US2022/034507

§ 371 Date: Dec. 21, 2023

Related U.S. Application Data
(60) Provisional application No. 63/213,417, filed on Jun. 22, 2021.

(51) Int. Cl. C07K 7/50; A61K 38/00; A61P 35/00

(52) U.S. Cl. CPC C07K 7/50; A61P 35/00; A61K 38/00

(57) ABSTRACT

Disclosed are Ras inhibiting bicyclic peptides. Also provided herein are methods of treating cancer in a subject, including human subject, and animal. The methods include administering to a subject an effective amount of one or more of the compounds or compositions described. The disclosed methods can optionally include identifying a patient who is or can be in need of treatment of a cancer.

### CROSS-REFERENCE TO RELATED APPLICATIONS

This application is a U.S. National Stage application filed under 35 U.S.C. § 371 of PCT/US2022/034507 filed Jun. 22, 2022, which claims the benefit of U.S. Provisional Appl. Ser. No. 63/213,417, filed Jun. 22, 2021, each of which is incorporated by reference as if fully set forth herein.

### ACKNOWLEDGEMENT OF GOVERNMENT SUPPORT

This invention was made with government support under grant/contract numbers R35 GM122459 and R01 CA234124 awarded by the National Institutes of Health. The government has certain rights in the invention.

### SEQUENCE LISTING

A Sequence Listing conforming to the rules of WIPO Standard ST.25 is hereby incorporated by reference. Said Sequence Listing has been filed as an electronic document via the USPTO patent electronic filing system in ASCII formatted text. The electronic document, created on Jan. 22, 2024, is entitled “103361-107WO1_ST25.txt”, and is 1,979 bytes in size.

### INCORPORATION BY REFERENCE OF SEQUENCE LISTING PROVIDED AS A TEXT FILE

A Sequence Listing is provided herewith as a text file, “2244605.txt” created on Jun. 6, 2022 and having a size of 1,607 bytes. The contents of the text file are incorporated by reference herein in their entirety.

### FIELD OF THE INVENTION

The invention is directed to cyclic peptides with therapeutic properties. In some embodiments, the compounds are bicyclic peptides. In some embodiments, the cyclic peptides can be used as Ras inhibitors, for example in the treatment of cancer, including solid tumors.

### BACKGROUND

The Ras subfamily of small GTPases consists of four isoforms, HRas, NRas, KRas4A and KRas4B. The four isoforms share 100% sequence identity in the effector lobe (residues 1-86), which engages effector proteins via their Switch I and Switch II motifs, and ˜86% sequence similarity within the allosteric lobe (residues 87-172) but diverge in the C-terminal hypervariable region (HVR). All isoforms possess a C-terminal CaaX motif which is farnesylated, leading to Ras accumulation on the plasma membrane. KRas4A and 4B also contain a polylysine stretch that interacts with the negatively charged phospholipids on the inner leaflet of the plasma membrane. The different C-terminal structures of the four isoforms result in differential membrane association and isoform-specific trafficking, impacting the biological activity of Ras isoforms.

The Ras GTPases function as molecular switches during receptor signaling in mammalian cells and cycle between a GTP-bound “On” state and a GDP-bound “Off” state.

Activation of Ras by an extracellular signal is mediated by guanine nucleotide exchange factors (GEFs), which enhance the rate of GDP dissociation by 10⁴-fold, enabling the incorporation of GTP, as GTP is present inside the cell at a 10-fold higher concentration than GDP. The activated Ras proteins interact with downstream effector proteins, including Raf kinases and phosphoinositide 3-kinase (PI3K), and activate their signaling cascades. Subsequent hydrolysis of the bound GTP to GDP, which is accelerated by 10³-fold in the presence of GTPase activating proteins (GAPs), inactivates the Ras proteins and terminates the signaling events.

Mutations of Ras active-site residues (e.g., Gly₁₂, Gly₁₃, and Gln₆₁) frequently slow down the GTP hydrolysis by reducing the intrinsic GTPase activity of Ras proteins and/or inhibiting the binding of GAPs to Ras proteins. This results in an excessive Ras-GTP population, leading to uncontrolled cell proliferation and survival, which are hallmarks of cancers. Indeed, mutations in Ras (including KRas, HRas, and NRas mutations) are found in ˜30% all human cancers, making Ras one of the most compelling anticancer drug targets. Unfortunately, Ras is also one of the most challenging targets, because it is intracellular and its surface has no major binding pocket for small molecules to bind. Nevertheless, small-molecule direct Ras inhibitors have recently been developed. Best results have, so far, been obtained with irreversible inhibitors that covalently modify (and inhibit) the KRas G₁₂C mutant, with one of the inhibitors (Lumakras) recently approved for treatment of non-small cell lung cancer (NSCLC) by the Food and Drug Administration. However, while the G₁₂C-specific inhibitors validate direct inhibition of Ras as a viable approach to treating Ras mutant cancers, the covalent inhibition approach is unlikely to be effective for other Ras mutants (e.g., G₁₂V, G₁₂S, G₁₂D, G₁₃D, and Q₆₁H).

Because of their larger sizes, proteins and peptides are capable of recognizing flat binding sites and have been exploited as Ras inhibitors. For example, several antibodies, monobodies, and an engineered Ras-binding domain (RBD) of CRAF have been developed as potent and highly specific Ras inhibitors in vitro; however, intracellular delivery of these large biomolecules remains a major challenge. A peptide inhibitor, cyclorasin B3, bound to GTP- or GppNHp-bound KRas with Kp values of 1.2 and 1.6 μM, respectively, and blocked the Ras-Raf interaction in vitro. Interestingly, cyclorasin B3 bound KRas-GDP with 8-fold lower affinity (KD=9.3 μM). Unfortunately (though not surprisingly), mono- and bicyclic peptides have poor membrane permeability and showed no to little cellular activity.

The remains a need for improved anti-cancer agents. There remains a need for improved Ras inhibitors. There remains a need for Ras inhibitors with increased cell permeability. There remains a need for Ras inhibitors with improved affinity for Ras-GTP relative to Ras-GDP.

### SUMMARY

In accordance with the purposes of the disclosed materials and methods, as embodied and broadly described herein, the disclosed subject matter, in one aspect, relates to compounds, compositions and methods of making and using compounds and compositions. In specific examples, the compounds have the formula:

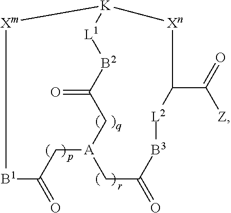

wherein
A is selected from N, aryl, C₃₋₈cycloalkyl, C₁₋₈ heterocyclyl, and C₁₋₈ heteroaryl;
p, q, and r are independently selected from 0, 1, and 2;
B¹, B², and B³ are independently selected from null, O and NR¹;
   wherein R¹ comprises H, or substituted or unsubstituted C₁-C₅ alkyl;
L¹ is selected from substituted or unsubstituted C₁-C₆ alkyl;
L² is selected from substituted or unsubstituted C₁-C₆ alkyl;
X^{m} and Xⁿ independently comprise a sequence of 1-10 amino acids;
K has a structure represented by formula:

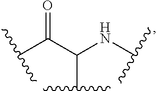 [SMILES: *NC(*)C(*)=O]

wherein each wavy indicates a point of attachment to one of X″ “, X”, and L¹; and Z represents is a sequence of 3-8 amino acids.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIGS. 1A and 1B depict the binding affinity of B4-27 to KRas. (FIG. 1A) HTRF assay showing inhibition of the interaction between KRasG12V-GppNHp (50 nM) and CRAF RBD (50 nM) by B4-27. (FIG. 1B) Binding of B4-27^{FAM} (100 nM) to KRasG12V loaded with GTPγS, GppNHp, or GDP as monitored by FP. Data reported are presented as mean±SD of 3 independent experiments.

FIGS. 2A-2D depict the cellular entry and intracellular distribution of B4-27. Flow cytometry analysis of HeLa cells after treatment with 1 (FIG. 2A) or 5 μM fluorescein-labeled CPP12 or B4-27 (FIG. 2B) for 2 h in the presence of 1% FBS. Values reported represent the mean±SD from 3 independent experiments (n=3) and relative to that of CPP12 (100%). (FIG. 2C) Live-cell confocal microscopic images of H358 cells after treatment with 3 μM B4-27^{FAM} for 2 h in the presence of 1% FBS. (FIG. 2D) Co-localization of B4-27^{TMR} (red) with GFP-KRasG12V (green) in MDCK cells stably expressing GFP-KRasG12V after treatment with 5 μM B4-27^{TMR} for 2 h in the presence of 1% FBS. White arrows, putative endosomes; yellow arrows, plasma membrane; scale bars, 5 μm.

FIGS. 3A and 3B depict the inhibition of Ras signaling by B4-27. (FIG. 3A) Dose-dependent inhibition of the interaction between KRasG12V or KRasG12D and CRAF RBD by B4-27 in HEK293T cells as monitored by the BRET2 assay. The BRET signals reported are relative to that of vehicle (DMSO)-treated cells (100%). (FIG. 3B) Western blots showing the effect of B4-27 on the phosphorylation levels of signaling proteins downstream of Ras (Akt, MEK). GAPDH was used as a loading control. Representative image from 4 independent experiments is shown.

FIGS. 4A-4C depict how B4-27 causes apoptotic death of Ras mutant cancer cell lines. (FIG. 4A) Effect of B4-27 on the viability of various cancer and non-cancerous cell lines as monitored by the Cell-Titer Glo™ 2.0 Viability Assay. Cells were incubated with indicated concentrations of B4-27 for 96 h in the presence of 10% FBS. (FIG. 4B) Annexin V/PI staining of H358 cells after treatment with indicated concentrations of B4-27 for 4 h in the presence of 10% FBS. Cytometry data were gated so that Q1 contained necrotic cells, Q2 contained late apoptotic cells, Q3 represented early apoptotic cells and Q4 were live cells. (FIG. 4C) Percentage of annexin V-positive cells (in Q2 and Q3) as a function of B4-27 concentration. Data shown represent the mean±SD from at least three independent experiments.

FIGS. 5A-5D depict the suppression of tumor growth by B4-27 in A549 mouse xenografts (n=6). (FIG. 5A) Tumor volume of B4-27- and vehicle-treated groups over 9 days. (FIG. 5B) The average tumor weight of B4-27- and vehicle-treated groups on the day of harvest. (FIG. 5C) Macroscopic images of the tumors resected from mice on the day of harvest. (FIG. 5D) The average mouse body weight of B4-27- and vehicle-treated groups over 9 days.

FIGS. 6A and 6B depict representative images of H&E staining, and immunohistochemical staining for p-ERK and Ki-67 in A549 (FIG. 6A) and H358 xenografts (FIG. 6B).

FIGS. 7A and 7B depict binding of B4-27^{FAM} (100 nM) to five arbitrarily selected proteins as monitored by FP. (FIG. 7A) BSA; (FIG. 7B) PNP, PTP1B, MDM2 and GST-RBD. Data shown represent the mean±SD of 3 independent experiments.

FIGS. 8A and 8B depicts live-cell confocal microscopic images of H358 (FIG. 8A), A549 (FIG. 8B), and HeLa cells (FIG. 8C) after treatment with 5 μM B4-27^{FAM} for 2 h in the presence of 1% FBS. I, FAM fluorescence; II, DIC. Scale bars, 20 μm.

FIGS. 9A-9C depicts live-cell confocal microscopic images of H358 cells after treatment with 5 M B4-27^{FAM} (FIG. 9A), B3-4^{FAM} (FIG. 9B), or B4^{FAM} (FIG. 9C) for 2 h in the presence of 1% FBS. I, FAM fluorescence; II, DIC. Scale bars, 20 μm.

FIG. 10 depicts Co-localization of B4-27^{TMR} (red) with GFP-KRasG12V (green) in MDCK cells stably expressing GFP-KRasG12V after treatment with 5 μM B4-27^{TMR} for 2 h in the presence of 1% FBS. I, GFP fluorescence; II, TMR fluorescence; III, Merge of I and II; IV, DIC. Scale bars, 20 μm.

FIG. 11A depicts lactate dehydrogenase (LDH) release assay. H358 cells were treated with increasing concentrations of B4-27 (0-20 μM), vehicle (RPMI+10% FBS), or cell lysis solution for 45 min and the amount of LDH activity in the growth medium was quantitated. (FIG. 11B) Stability of B4-27 in human serum. B4-27 (100 μM) was incubated in 25% human serum in DPBS (v/v) at 37° C. and 50-μL aliquots were withdrawn at the indicated time points, mixed with 15% TCA in MeOH: MeCN, and analyzed by RP-HPLC. The amount of intact B4-27 remaining (relative to that at 0 h; 100%) was plotted as a function of incubation time. Data reported represent the mean±SD of three independent experiments.

FIGS. 12A-12D depicts suppression of tumor growth in H358 mice xenografts by B4-27 (n=7). (FIG. 12A) Tumor volume of vehicle- and B4-27-treated groups over 9 days. (FIG. 12B) The average tumor weight of vehicle- and B4-27-treated groups on the day of harvest. (FIG. 12C) Representative macroscopic images of the tumors resected from mice on the day of harvest. (FIG. 12D) The average mice body weight of vehicle- and B4-27-treated groups over 9 days.

FIG. 13 depicts the chemical structure of B4-27.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes ¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

A residue of a chemical species, as used herein, refers to a derivative of a moiety that is present in a particular product. To form the product, at least one atom of the moiety is replaced by a bond to a second moiety, such that the product contains a derivative of a moiety. For example, in some embodiments, an aromatic residue in a product may refer to one or more-(C₆H5), units present in a cyclic peptide described herein. Similarly, an amino acid residue in a product may refer to cyclic peptide described herein having an amino acid incorporated therein through formation of one or more peptide bonds, and such residues may be referred to interchangeably herein as an amino acid or an amino acid residue.

As used herein, the term “chirality” refers to the “D” and “L” isomers of amino acids or amino acid residues. In generic form, an α-L-amino acid has the following configuration:

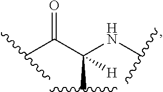 [SMILES: *N[C@@H](*)C(*)=O]

while an α-D-amino acid will have the opposite configuration.

As used herein, the term “non-aromatic hydrophobic” refers to a moiety that is not soluble in water and which does not comprise an aromatic ring. Generally, neutral moieties and/or non-polar moieties, or moieties that are predominately neutral and/or non-polar are hydrophobic.

Hydrophobicity can be measured by one of the methods disclosed herein below. Non-aromatic hydrophobic residues include saturated and unsaturated carbocyclyl and heterocyclyl groups which are not aromatic, as well as alkyl, alkenyl, and alkynyl. In some embodiments, the term “non-aromatic hydrophobic” can include groups in which a hydrophobic residue to attached to rest of the molecule through a bonding group which otherwise could be considered to be polar, such as acyl and alkylcarboxamidyl groups as defined below.

As used herein “aromatic” refers to an unsaturated cyclic molecule having 4n+2 π electrons, wherein n is any integer. The term “non-aromatic” refers to any unsaturated cyclic molecule which does not fall within the definition of aromatic.

The term “acyl” refers to groups-C(O)R, where R is hydrogen, alkyl, alkenyl, alkynyl, carbocyclyl, or heterocyclyl, as defined herein. Unless stated otherwise specifically in the specification, acyl can be optionally substituted.

“Alkyl” or “alkyl group” refers to a fully saturated, straight or branched hydrocarbon chain radical having from one to forty carbon atoms, and which is attached to the rest of the molecule by a single bond. Alkyls comprising any number of carbon atoms from 1 to 20 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈ alkyl. A C₁-C₈ alkyl includes C₅ alkyls, C₄ alkyls, C₃ alkyls, C₂ alkyls and C₁ alkyl (i.e., methyl). A C₁-C₆ alkyl includes all moieties described above for C₁-C₈ alkyls but also includes C₆ alkyls. A C₁-C₁₀ alkyl includes all moieties described above for C₁-C₈ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Alkenyl group comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes Cn and C₁₂ alkenyls. Non-limiting examples of C₂-C₁₂ alkenyl include ethenyl(vinyl), 1-propenyl, 2-propenyl(allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” refers to the group —OR, where R is alkyl, alkenyl, alkynyl, cycloalkyl, or heterocyclyl as defined herein. Unless stated otherwise specifically in the specification, alkoxy can be optionally substituted.

“Alkylcarbamoyl” refers to the group —O—C(O)—NRₐR_{b}, where Rₐ and R_{b} are the same or different and independently an alkyl, alkenyl, alkynyl, aryl, heteroaryl, cycloalkyl, or heterocyclyl group, as defined herein, or RₐR_{b} can be taken together to form a heterocyclyl group, as defined herein. Unless stated otherwise specifically in the specification, alkylcarbamoyl can be optionally substituted.

“Alkylcarboxamidyl” refers to the group —C(O)—NRₐR_{b}, where Rₐ and R_{b} are the same or different and independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, alkylcarboxamidyl can be optionally substituted.

“Alkoxycarbonyl” refers to the group —C(O) OR, where R is alkyl, alkenyl, alkynyl, aryl, heteroaryl, cycloalkyl, cycloalkenyl, cycloalkynyl, or heterocyclyl group, as defined herein. Unless stated otherwise specifically in the specification, alkoxycarbonyl can be optionally substituted.

“Alkylthio” refers to the —SR or —S(O)ₙ₌₁₋₂—R, where R is alkyl, alkenyl, alkynyl, cycloalkyl, cycloalkenyl, cycloalkynyl, or hetereocyclyl, as defined herein. Unless stated otherwise specifically in the specification, alkylthio can be optionally substituted.

“Arylthio” refers to the —SR or —S(O)ₙ₌₁₋₂—R, where R is aryl or hetereoaryl, as defined herein. Unless stated otherwise specifically in the specification, arylthio can be optionally substituted.

“Alkynyl” or “alkynyl group” refers to a straight or branched hydrocarbon chain radical having from two to forty carbon atoms, and having one or more carbon-carbon triple bonds. Each alkynyl group is attached to the rest of the molecule by a single bond. Alkynyl group comprising any number of carbon atoms from 2 to 40 are included. An alkynyl group comprising up to 40 carbon atoms is a C₂-C₄₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain, having from two to forty carbon atoms, and having one or more carbon-carbon triple bonds. Non-limiting examples of C₂-C₄₀ alkynylene include ethynylene, propargylene and the like. Unless stated otherwise specifically in the specification, an alkynylene chain can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Unless stated otherwise specifically in the specification, the carbocyclyl can be a monocyclic, bicyclic, tricyclic or tetracyclic ring system, which can include fused or bridged ring systems Carbocyclic rings include cycloalkyl, cycloalkenyl, and cycloalkynyl as defined herein. In some embodiments, the carbocyclyl is monovalent and is attached to the rest of molecule through a single bond. In some embodiments, the carbocyclyl is divalent and is independently attached to two moieties through single bonds. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Heterocyclyl,” “heterocyclic ring” or “heterocycle” refers to a stable 3- to 20-membered non-aromatic ring radical, which consists of two to fourteen carbon atoms and from one to eight heteroatoms selected from the group consisting of nitrogen, oxygen and sulfur. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl is monovalent and is attached to the rest of molecule through a single bond. In some embodiments, the heterocyclyl is divalent and is independently attached to two moieties through single bonds. Unless stated otherwise specifically in the specification, a heterocyclyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aryloxy” refers to groups-OAr, where Ar is an aryl or heteroaryl group as defined herein. Unless otherwise stated specifically in the specification, the aryloxy group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Aralkyl” refers to a radical of the formula —R_{b}—R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)OR, —NR_{g}SO₂Rₕ, —OC(═O) NR_{g}Rₕ, —OR_{g}, —SR_{g}, —SOR_{g}, —SO₂R_{g}, —OSO₂R_{g}, —SO₂OR_{g}, ═NSO₂R_{g}, and ═SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Unless stated to the contrary, a substituent drawn without explicitly specifying the point of attachment indicates that the substituent may be attached at any possible atom. For example, in a benzofuran depicted as:

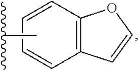

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

As used herein, the term “about” or “approximately” refers to a quantity, level, value, number, frequency, percentage, dimension, size, amount, weight or length that varies by acceptable levels in the art. In some embodiments, the amount of variation may be as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

A numerical range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

The terms “peptide”, “polypeptide”, and “protein” are used interchangeably herein, and refer to a polymeric form of amino acids of any length, which can include coded and non-coded amino acids, chemically or biochemically modified or derivatized amino acids, and polypeptides having modified peptide backbones. The term “modified” refers to a substance or compound (e.g., a cell, a polynucleotide sequence, and/or a polypeptide sequence) that has been altered or changed as compared to the corresponding unmodified substance or compound.

The term “non-natural amino acid” (or “unnatural amino acid”) refers to an organic compound that is a congener of a natural amino acid in that it has an amine (—NH₂) group on one end and a carboxylic acid (—COOH) group on the other end but the side chain or backbone is modified. The resulting moiety has a structure and reactivity that is similar but not identical to a natural amino acid. Non-limiting examples of such modifications include elongation of the side chain by one or more methylene groups, replacing one atom with another, and increasing the size of an aromatic ring. The non-natural amino acid can be a modified amino acid, and/or amino acid analog, that is not one of the 20 common naturally occurring amino acids or the rare natural amino acids selenocysteine or pyrrolysine. For example, an analog of arginine may have one more or one few methylene group on the side chain. Non-natural amino acids can also be the D-isomer of the natural amino acids. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, naphthylalanine, phenylalanine, proline, pyroglutamic acid, serine, threonine, tryptophan, tyrosine, valine, a derivative, or combinations thereof.

Amino acids can be designated by a three or one letter code, for example as shown in the table below:

| Amino Acid | Abbreviations* |
|---|---|
| alanine | Ala (A) |
| allosoleucine | Alle |
| arginine | Arg (R) |
| asparagine | Asn (N) |
| aspartic acid | Asp (D) |
| cysteine | Cys (C) |
| glutamic acid | Glu (E) |
| glutamine | Gln (Q) |
| glycine | Gly (G) |
| histidine | His (H) |
| isoleucine | Ile (I) |
| leucine | Leu (L) |
| lysine | Lys (K) |
| Methionine | Met (M) |
| napthylalanine | Nal(Q) |
| phenylalanine | Phe (F) |
| proline | Pro (P) |
| selenocysteine | Sec (U) |
| serine | Ser (S) |
| threonine | Thr (T) |
| tyrosine | Tyr (Y) |
| tryptophan | Trp (W) |
| valine | Val (V) |
| Phenylglycine | Phg |
| Propargylglycine | Pra |

As used herein, an amino acid code with a capital letter refers to the L-amino acid. A D-amino acid is described when the code is present as a lower case letter, or as a three letter code composed entirely of lower case letters.

Disclosed herein are peptidyl compounds, pharmaceutical formulations of such compounds, and methods of using such compounds as Ras inhibitors. By selectively binding Ras-GTP (over Ras-GDP), interaction with effector proteins is blocked, inducing apoptosis of Ras mutant cancer cells in vitro, and suppressing tumor growth.

Disclosed herein are compounds having the formula:

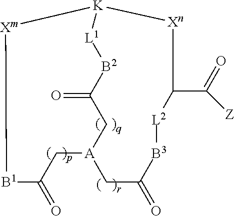

wherein
A is selected from N, aryl, C₃₋₈cycloalkyl, C₁₋₈ heterocyclyl, and C₁₋₈ heteroaryl;
p, q, and r are independently selected from 0, 1, and 2;
B¹, B², and B³ are independently selected from null, O and NR¹;
   wherein R¹ comprises H, or substituted or unsubstituted C₁-C₅ alkyl;
L¹ is selected from substituted or unsubstituted C₁-C₆ alkyl;
L² is selected from substituted or unsubstituted C₁-C₆ alkyl;
X^{m} and Xⁿ independently comprise a sequence of 1-10 amino acids;
K has a structure represented by formula:

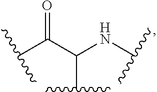 [SMILES: *NC(*)C(*)=O]

wherein each wavy indicates a point of attachment to one of X^{m}, Xⁿ, and L¹; and
   Z represents a sequence of 3-8 amino acids, OR^{π}, or NHR^{π},
   wherein R™ is selected from H, —(CH₂CH₂O)ₛH, and s is 1-100.
   In some examples Z can be an amino acid having the sequence:

(SEQ ID NO: 1)
*NH-X⁷-X⁶-X⁵-X⁴-X³-X²-X¹-X⁰-C(O)-X^{z},

wherein the asterisk represents the point of attachment to the compound of Formula 1,
X^{z} is O R^{π} or NHR^{π};
X⁷ is selected from null, a natural, or non-natural amino acid;
X⁶ is selected from null, a natural, or non-natural amino acid;
X⁵ is selected from null, a natural, or non-natural amino acid;
X⁴ is selected from null, a natural, or non-natural amino acid;
X³ is selected from a natural, or non-natural amino acid;
X² is selected from a natural, or non-natural amino acid;
X¹ is selected from a natural, or non-natural amino acid; and X⁰ is selected from null, a natural, or non-natural amino acid,
wherein R^{π} is as defined above.

In certain embodiments, X⁰ is selected from null, glycine, D-alanine, L-alanine, D-lysine, or L-lysine.

In certain embodiments, X¹ is selected from glycine, D-alanine, L-alanine, D-threonine, L-threonine, D-homoalanine, L-homaalanine, D-valine, L-valine, D-leucine, L-leucine, D-isoleucine, L-isoleucine, β-alanine, D-tert-butyl-alanine, L-tert-butyl-alanine, D-tert-butyl-glycine, L-tert-butyl-glycineine, D-methyleucine, and L-methylleucine.

In certain embodiments, X² is selected from glycine, D-alanine, L-alanine, L-phenylalanine, D-phenylalanine, L-homophenyl alanine, D-homophenyl alanine, L-3-chlorophenylalanine, D-3-chlorophenylalanine, L-phenylglycine, D-phenylglycine, L-3,4-difluorophenylalanine, L-3-cyclohexylalanine, L-3-(2-pyridyl)-alanine, D-3,4-difluorophenylalanine, D-3-cyclohexylalanine, D-3-(2-pyridyl)-alanine, L-arginine, or D-arginine.

In certain embodiments, X³ is selected from L-arginine, D-arginine, L-homoarginine, or D-homoarginine.

In certain embodiments, X⁴ is selected from D-ornithine, L-ornithine, D-lysine, L-lysine, D-arginine, L-arginine, D-asparagine, L-asparagine, D-glutamine, L-glutamine.

In certain examples, Z is an amino acid sequence having the formula:

(SEQ ID NO: 2)
*NH-X⁴-X³-X²-X¹-X⁰-C(O)-X^{z},

wherein the asterisk represents the point of attachment to the compound of Formula 1;
X² is OH or NH₂;
X⁴ is selected from L-ornithine, D-ornithine, L-lysine, or D-lysine;
X³ is D-arginine;
X² is selected from L-phenylalanine, D-phenylalanine, L-phenylglycine, or D-phenylglycine; and
X¹ is selected from L-alanine, D-alanine, L-threonine, D-threonine, or glycine.

In certain examples, Z is an amino acid sequence having the formula:

(SEQ ID NO: 3)
*NH-X⁴-X³-X²-X¹-C(O)-X^{z},

wherein the asterisk represents the point of attachment to the compound of Formula 1;
X² is OH or NH₂;
X⁴ is L-ornithine;
X³ is D-arginine;
X² is L-phenylalanine; and
X¹ is D-alanine, i.e., a sequence having the formula:

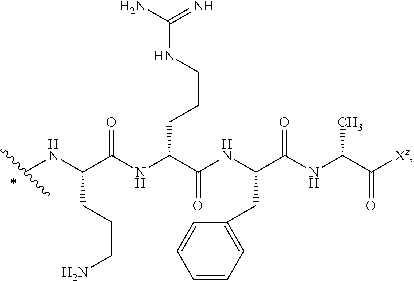

In other embodiments X⁴ is L-ornithine, X³ is D-arginine, X² is L-phenylalanine, and X¹ is D-threonine.

Exemplary A groups include phenyl, cyclohexyl, cyclopropyl, cyclopentyl, pyridinyl, pyrimidinyl, and imidazolyl. Non limiting examples include:

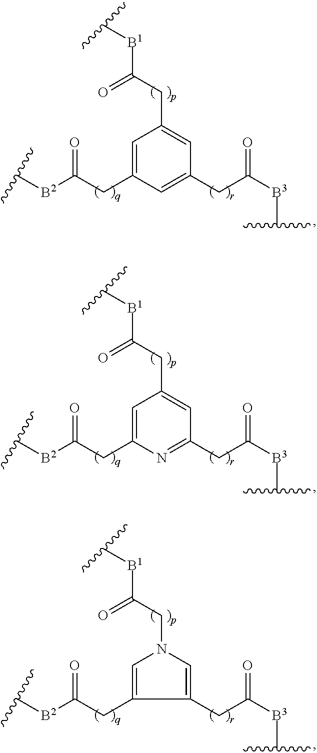

In certain embodiments, the compound of Formula I has the formula:

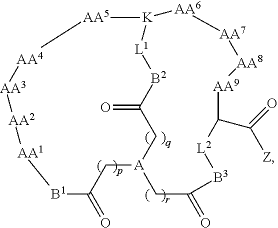

wherein A, p, q, r, B¹, B², B³, L¹, L², K, and Z are as defined above, and AA¹, AA², AA³, AA⁴, AA⁵, AA⁶, AA⁷, AA⁸, and AA⁹ are each independently selected from a natural or non-natural amino acid.

In certain embodiments, AA¹ is selected from D-naphtylalanine, L-naphtylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphtylglycine, L-naphtylglycine, D-cyclohexylalanine, and L-cyclohexylalanine.

In certain embodiments, AA² is selected from D-serine, L-serine, D-threonine, L-threonine, D-cysteine, L-cysteine, D-methionine, and L-methionine.

In certain embodiments, AA³ is selected from D-glutamine, L-glutamine, D-asparagine, and L-asparagine.

In certain embodiments, AA⁴ is selected from D-naphtylalanine, L-naphtylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphtylglycine, L-naphtylglycine, D-cyclohexylalanine, and L-cyclohexylalanine.

In certain embodiments, AA⁵ is selected from L-phenylglycine, D-phenylglycine, D-naphtylalanine, L-naphtylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphtylglycine, L-naphtylglycine, D-cyclohexylalanine, L-cyclohexylalanine, D-tert-butylglycine, L-tert-butylglycine, D-valine, L-valine, D-leucine, L-leucine, D-alanine, L-alanine, D-isoleucine, L-isoleucine.

In certain embodiments, AA⁶ is L-phenylglycine, D-phenylglycine, D-naphtylalanine, L-naphtylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphtylglycine, L-naphtylglycine, D-cyclohexylalanine, L-cyclohexylalanine, D-tert-butylglycine, L-tert-butylglycine, D-valine, L-valine, D-leucine, L-leucine, D-alanine, L-alanine, D-isoleucine, L-isoleucine.

In certain embodiments, AA⁷ is selected from D-glutamine, L-glutamine, D-homoglutamine, L-homoglutamine, D-asparagine, and L-asparagine.

In certain embodiments, AA⁸ is selected from D-arginine, L-arginine, D-histidine, L-histadine, D-lysine, L-lysine, D-ornithine, L-ornithine, D-2,3-diaminopropionic acid, L-2,3-diaminopropionic acid, D-homoarginine, L-homoarginine, D-homolysine, L-homolysine, D-2,4-diaminobutyric acid, and L-2,4-diaminobutyric acid.

In certain embodiments, AA⁹ is selected from D-arginine, L-arginine, D-histidine, L-histadine, D-lysine, L-lysine, D-ornithine, L-ornithine, D-2,3-diaminopropionic acid, L-2,3-diaminopropionic acid, D-homoarginine, L-homoarginine, D-homolysine, L-homolysine, D-2,4-diaminobutyric acid, and L-2,4-diaminobutyric acid.

In some embodiments, each of AA¹, AA², AA³, AA⁴, and AA⁵ are L-amino acids. In certain embodiments, AA⁶ and AA⁸ are L-amino acids. In some embodiments, each of AA¹, AA², AA³, AA⁴, AA⁵, AA⁶, and AA⁸ are L-amino acids. In certain embodiments, AA⁷ and AA⁹ are D-amino acids.

In certain embodiments, AA¹ is selected from L-naphtylalanine, L-phenylalanine, L-4-fluorophenylalanine, L-3,4-difluorophenylalanine, L-2,4-difluorophenylalanine, L-cyclohexylglycine, L-naphtylglycine, and L-cyclohexylalanine. In further embodiments, AA¹ is selected from L-naphtylalanine, and L-naphtylglycine.

In certain embodiments, AA² is selected from L-serine, L-threonine, L-cysteine, and L-methionine. In certain embodiments, AA² is selected from L-serine and L-threonine,

In certain embodiments, AA³ is selected from L-glutamine and L-asparagine.

In certain embodiments, AA⁴ is selected from L-naphtylalanine, L-phenylalanine, L-4-fluorophenylalanine, L-3,4-difluorophenylalanine, L-2,4-difluorophenylalanine, L-cyclohexylglycine, L-naphtylglycine, and L-cyclohexylalanine. In further embodiments, AA⁴ is selected from L-naphtylalanine, and L-naphtylglycine.

In certain embodiments, AA⁵ is selected from L-phenylglycine, L-phenylalanine, L-cyclohexylglycine, L-cyclohexylalanine.

In certain embodiments, AA⁶ is D-phenylalanine.

In certain embodiments, AA⁷ is selected from D-glutamine, D-homoglutamine, and D-asparagine.

In certain embodiments, AA⁸ is selected from L-arginine, L-histadine, and L-homoarginine.

In certain embodiments, AA⁹ is selected from D-arginine, D-histidine, D-lysine, D-ornithine, D-2,3-diaminopropionic acid, D-homoarginine, D-homolysine, and D-2,4-diaminobutyric acid.

In certain embodiments, L¹ is an unsubstituted n-butylene chain and L² is an unsubstituted methylene chain. In other embodiments, L¹ is an unsubstituted n-butylene chain and L² is an unsubstituted n-butylene chain. In further embodiments, L¹ is an unsubstituted methylene chain and L² is an unsubstituted n-butylene chain. In other embodiments, L¹ is an unsubstituted methylene chain and L² is an unsubstituted methylene chain.

In some examples the compound of Formula 1 has the formula:

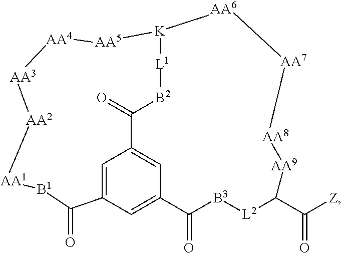

wherein B¹, B², B³, L¹, L², K, Z, AA¹, AA², AA³, AA⁴, AA³, AA⁶, AA⁷, AA⁸, and AA⁹ are as defined above.

In some examples the compound of Formula 1 has the formula:

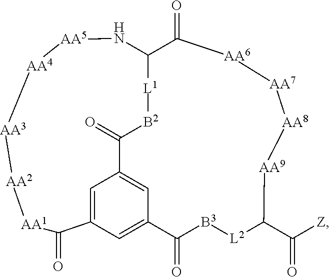

wherein B², B³, L¹, L², Z, AA¹, AA², AA³, AA¹, AA³, AA⁶, AA⁷, AA⁸, and AA⁹ are as defined above.

In some examples the compound of Formula 1 has the formula:

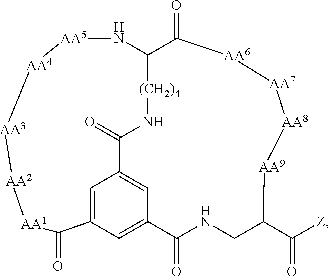

wherein Z, AA¹, AA², AA³, AA⁴, AA⁵, AA⁶, AA⁷, AA⁸, and AAP are as defined above.

In some examples the compound of Formula 1 has the formula:

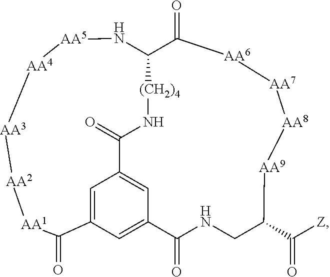

wherein Z, AA¹. AA², AA³, AA⁴, AA⁵, AA⁶, AA⁷. AA⁸, and AA⁹ are as defined above.

In certain examples of Formula I, the compounds are of Formula 1-C:

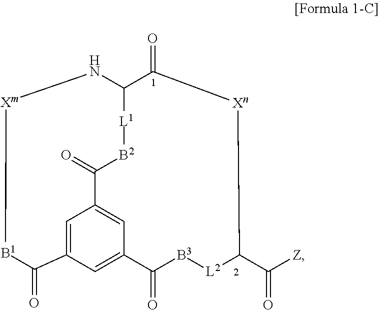

wherein Xⁿ has the formula:

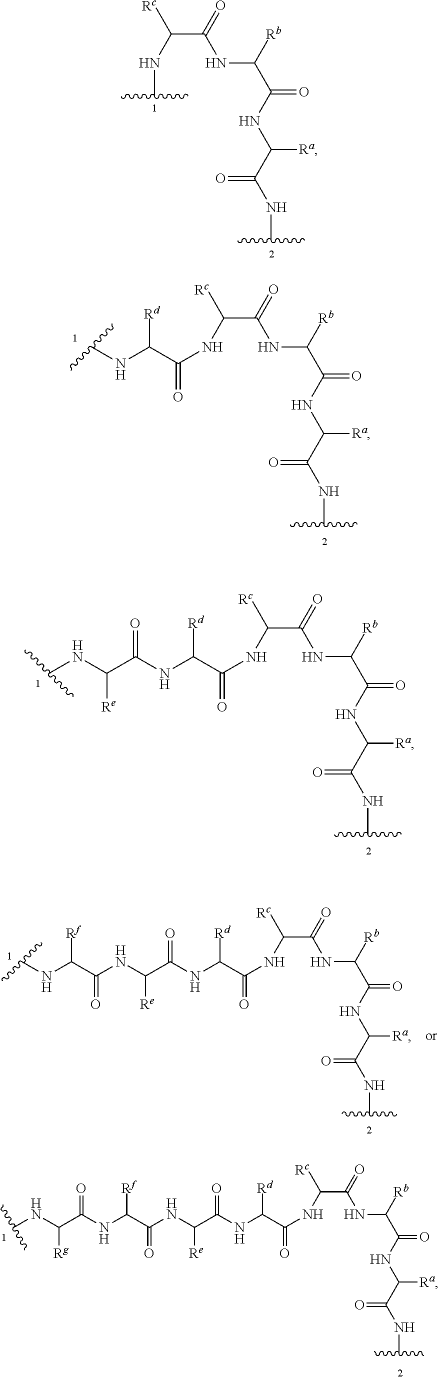

wherein wavy line I indicates the point of attachment to carbon 1 in Formula 1-C, wavy line 2 indicates the point of attachment to carbon 2 in Formula 1-C.

R^{a} has the formula:

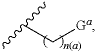

wherein n(a) is from 0-6, and G^{a} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, or CH(OH)CH₃.

In some instances G^{a} is methyl, isopropyl, n-butyl, n-propyl, 3-methylprop-1-yl, 2-methylprop-1-yl, 1-napthyl, phenyl, 2-napthyl, 3-napthyl, or cyclohexyl.

In some instances G^{a} has the formula:

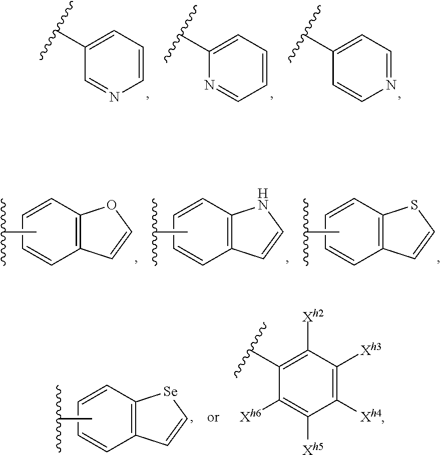

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In some embodiments, n(a) is 1, 2, or 3, and G^{a} is CONH₂, NH₂, NHC(═NH)NH₂, preferably NHC(═NH)NH₂.

R_{b} has the formula:

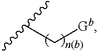

wherein n(b) is from 0-6, and G^{b} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{b} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{b} has the formula:

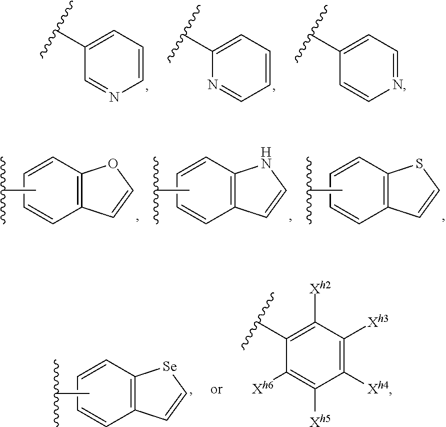

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In some embodiments, n(b) is 1, 2, or 3, and G^{b} is CONH₂, NH₂, NHC(═NH)NH₂, preferably NHC(═NH)NH₂.

R^{c} has the formula:

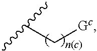

wherein n(c) is from 0-6, and G^{c} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{c} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{c} has the formula:

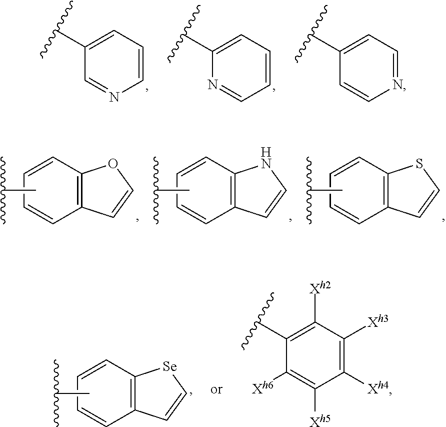

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain embodiments, n(c) is 1, 2, or 3, and G^{b} is CONH₂, NH₂, NHC(═NH)NH₂, preferably CONH₂.

R^{d} has the formula:

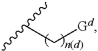

wherein n(d) is from 0-6, and G^{d} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{d} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{d} has the formula:

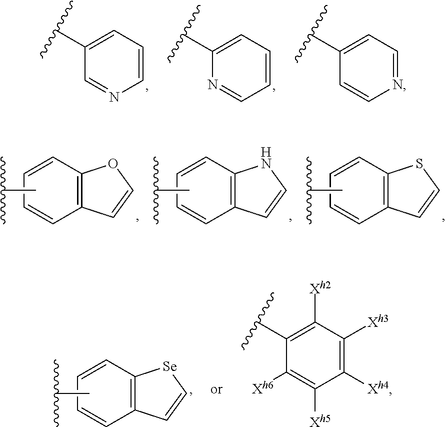

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain embodiments, n(d) is 0 or 1, and G^{d} is has the formula:

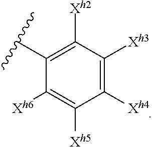

In some embodiments, X^{h2}, X^{h3}, X^{h4}, X^{h5}, and X^{h6} are each hydrogen. In other embodiments, X^{h4} is F or Cl, and X^{h2}, X^{h3}, X^{h5}, and X^{h6} are each hydrogen. In other embodiments, X^{h3} is F or Cl, X^{h4} is F or Cl, and X^{h2}, X^{h5}, and X^{h6} are each hydrogen.

R^{c} has the formula:

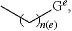

wherein n(e) is from 0-6, and G^{e} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{e} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{e} has the formula:

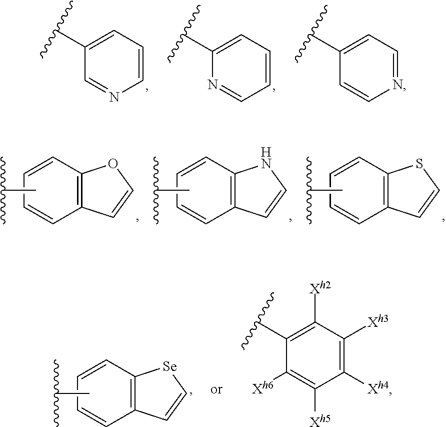

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

R^{f} has the formula:

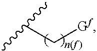

wherein n(b) is from 0-6, and G^{f} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{f} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{f} has the formula:

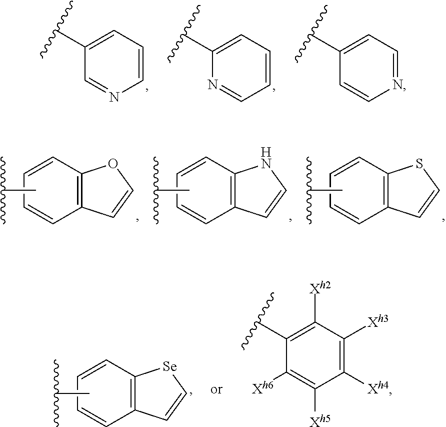

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

R⁸ has the formula:

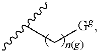

wherein n(g) is from 0-6, and G^{g} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{g} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{g} has the formula:

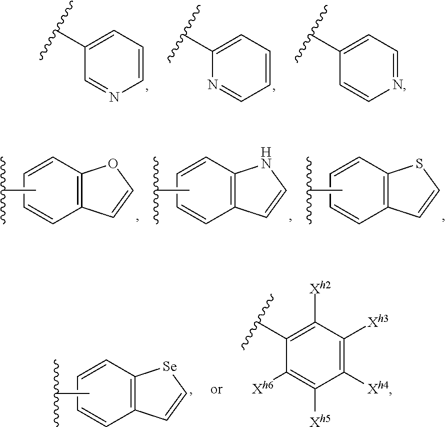

wherein:
X^{h2} is selected from H, OH, F, or Cl;
X^{h3} is selected from H, OH, F. or Cl;
X^{h4} is selected from H, OH, F, or Cl;
X^{h5} is selected from H, OH, F, or Cl;
X^{h6} is selected from H, OH, F, or Cl.

In certain examples of Formula I, the compounds are of Formula 1-D:

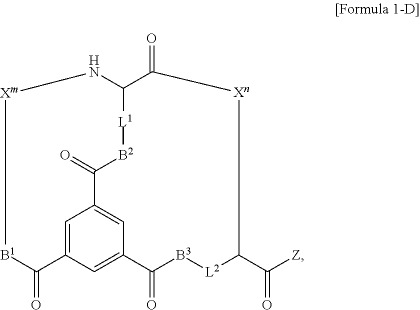

wherein X^{m} has the formula:

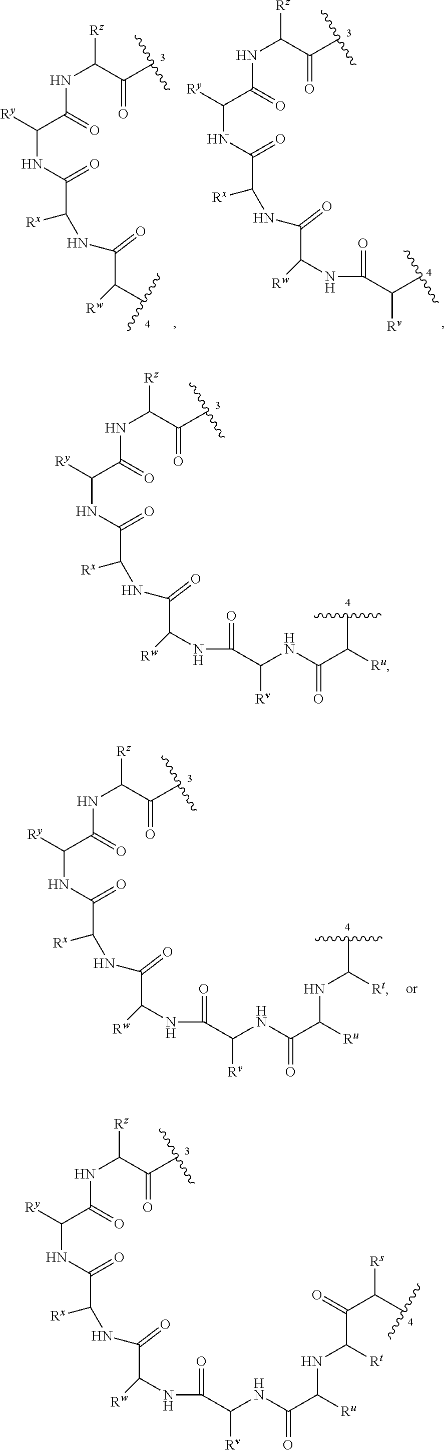

wherein wavy line 3 indicates the point of attachment to nitrogen 3 in Formula 1-D, wavy line 4 indicates the point of attachment to B¹,

R^{z} has the formula:

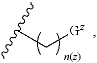

wherein n(z) is from 0-6, and G^{z} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, Or CH(OH)CH₃.

In some instances G^{z} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{z} has the formula:

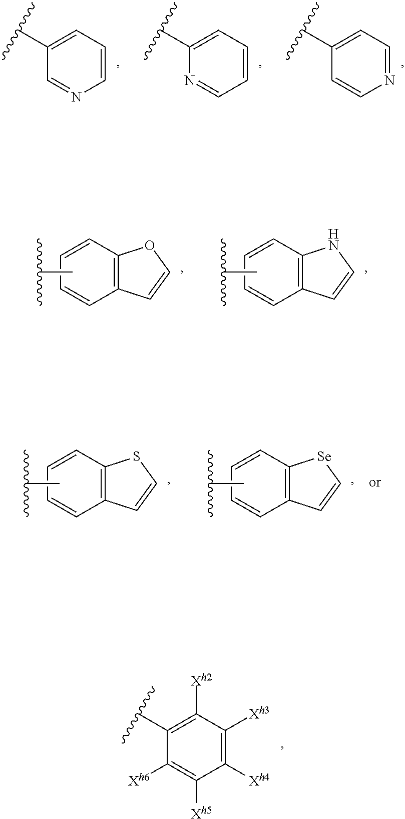

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain embodiments, n(z) is 0 or 1, and G^{z} is has the formula:

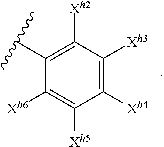

In some embodiments, X^{h2}, X^{h3}, X^{h4}, X^{h5}, and X^{h6} are each hydrogen. In other embodiments, X^{h4} is F or Cl, and X^{h2}, X^{h3}, X^{h5}, and X^{h6} are each hydrogen. In other embodiments, X^{h3} is F or Cl, X^{h4} is F or Cl, and X^{h2}, X^{h5}, and X^{h6} are each hydrogen.

R^{y} has the formula:

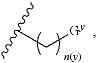

wherein n(y) is from 0-6, and G^{y} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{y} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{y} has the formula:

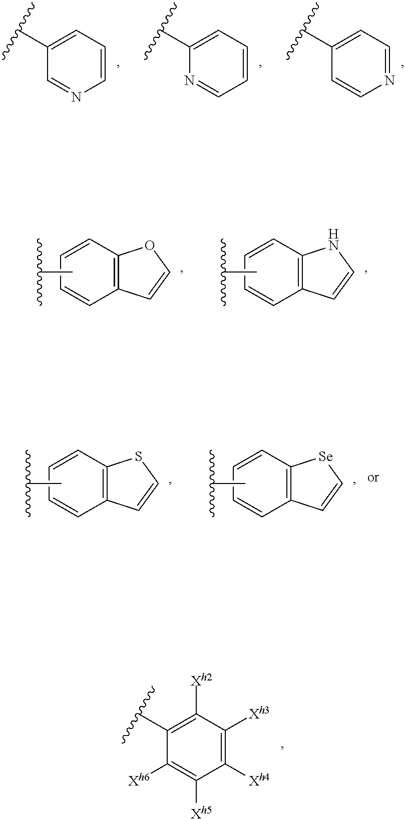

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain embodiments, n(y) is 0 or 1. In further embodiments, G^{y} has the formula:

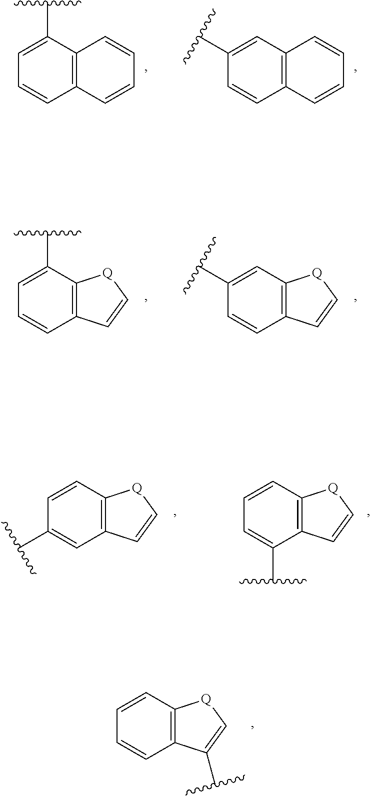

wherein Q is O, NH, S, or Se.

R^{x} has the formula:

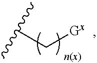

wherein n(x) is from 0-6, and G^{x} is hydrogen, C₁₋₆alkyl, C₃-cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{x} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{x} has the formula:

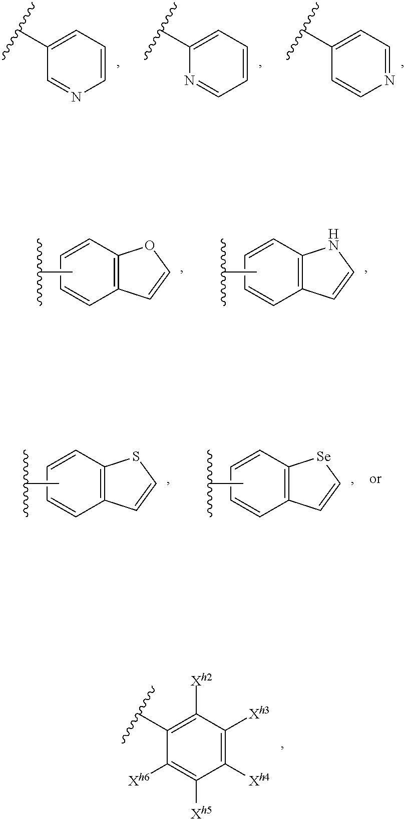

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain embodiments, n(c) is 1, 2, or 3, and G^{e} is COOH, C(O) NH₂, or NHC(═NH)NH₂.

R″ has the formula:

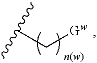

wherein n(w) is from 0-6, and G^{w} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{w} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{w} has the formula:

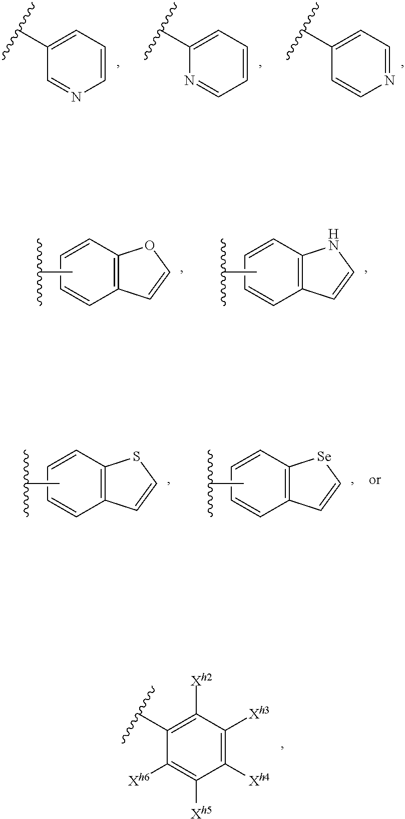

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain embodiments, n(w) is 0 and G^{w} is CH(CH₃)OH. In other embodiments, N (w) is 1, and G^{w} is OH.

R^{v} has the formula:

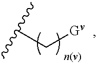

wherein n(v) is from 0-6, and G^{v} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{v} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{v} has the formula:

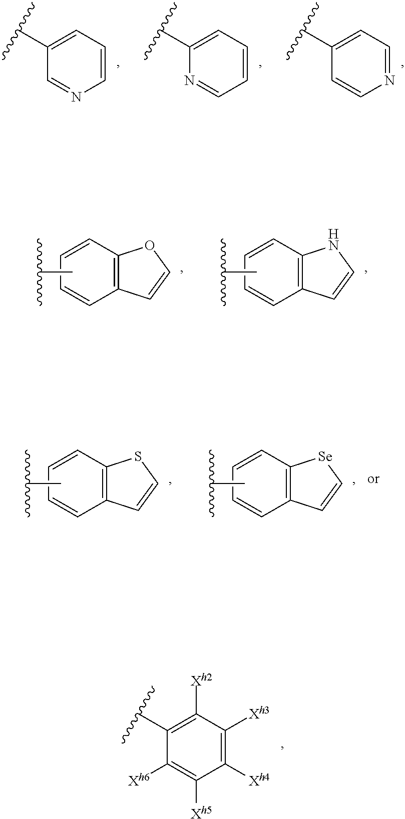

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain embodiments, n(v) is 0 or 1. In further embodiments, G′ has the formula:

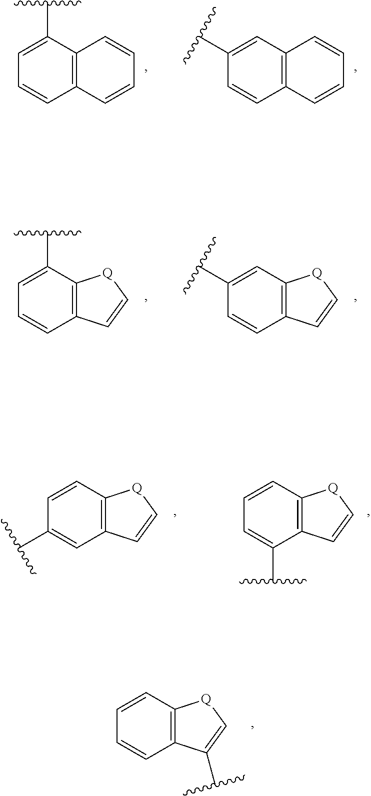

wherein Q is O, NH, S, or Se.

R″ has the formula:

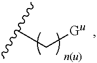

wherein n(u) is from 0-6, and G^{u} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{u} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{u} has the formula:

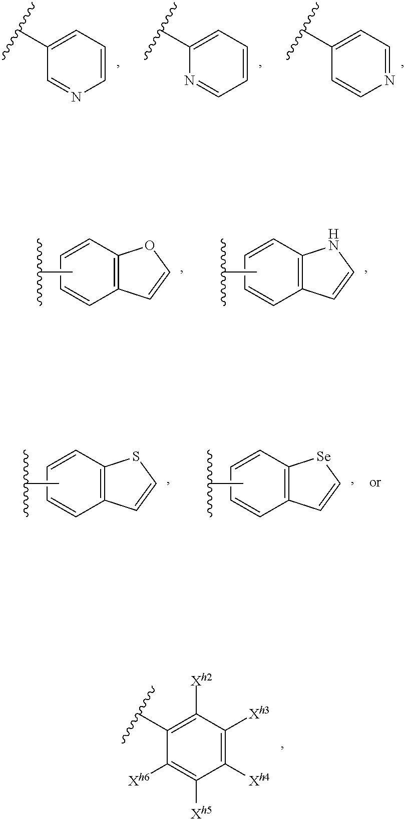

wherein:
X¹² is selected from H, OH, F, or Cl;
X¹³ is selected from H, OH, F, or Cl;
X^{h4} is selected from H, OH, F, or Cl;
X^{h5} is selected from H, OH, F, or Cl;
X^{h6} is selected from H, OH, F, or Cl.

R^{t} has the formula:

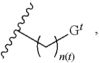

wherein n(t) is from 0-6, and G^{t} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{t} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{t} has the formula:

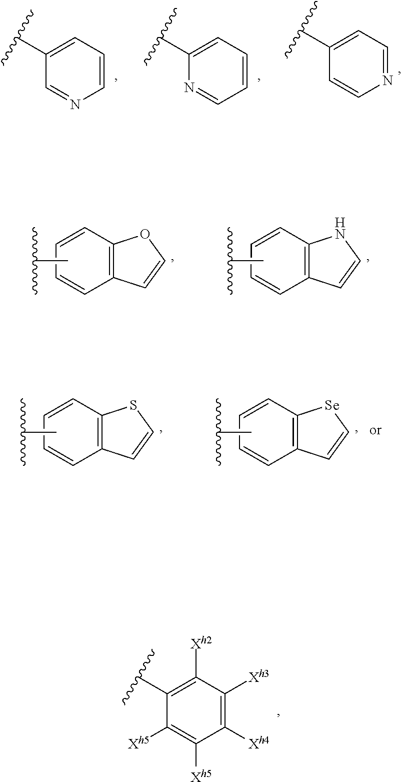

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

R^{s} has the formula:

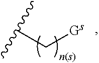

wherein n(s) is from 0-6, and G^{s} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

In some instances G^{s} is methyl, isopropyl, n-butyl, n-propyl, 3-methylprop-1-yl, 2-methylprop-1-yl, 1-napthyl, 2-napthyl, 3-napthyl, or cyclohexyl.

In some instances G^{s} has the formula:

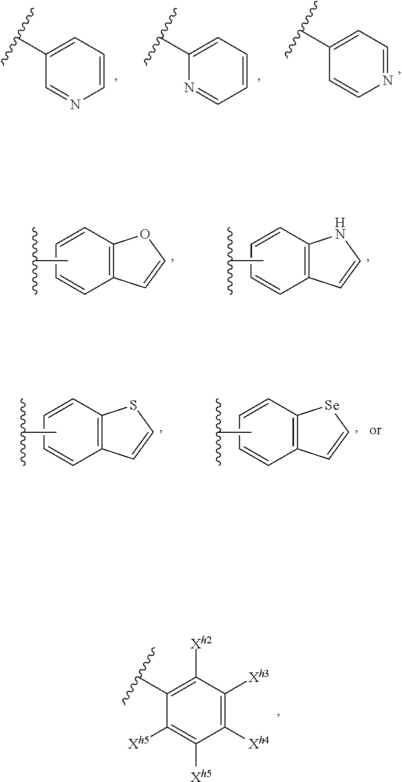

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

In certain examples of Formula I, the compounds are of Formula 1-D:

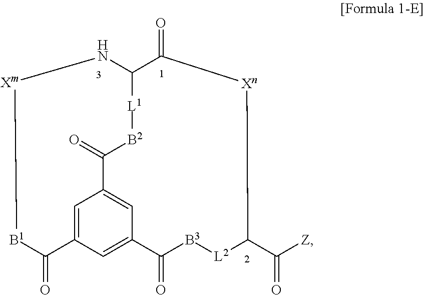

wherein Xⁿ and X^{m} are as defined above.

In some instances, X^{m} is a four amino acid sequence having the formula:

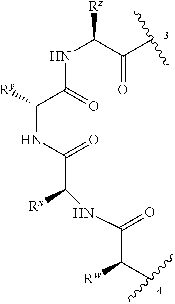

or a five amino acid sequence having the formula:

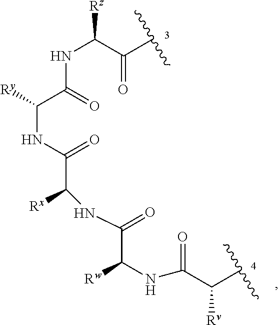

or a six amino acid sequence having the formula:

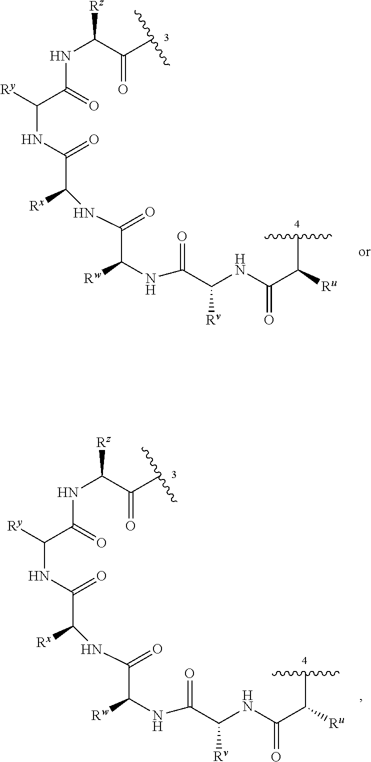

wherein wavy line 3, wavy line 4, r^{u}, r^{v}, r^{w}, r^{x}, r^{y}, and r^{z} are as defined above.

In certain embodiments, X^{m} has the formula:

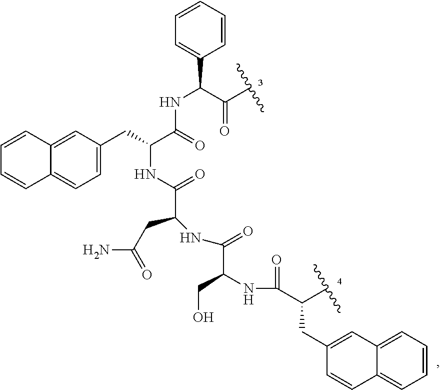 [SMILES: *C(=O)[C@@H](NC(=O)[C@@H](Cc1ccc2ccccc2c1)NC(=O)[C@H](CC(N)=O)NC(=O)[C@H](CO)NC(=O)[C@H](*)Cc1ccc2ccccc2c1)c1ccccc1]

wherein in wavy line 3 and wavy line 4 have the meanings given above.

In some embodiments, Xⁿ is a three amino acid sequence having the formula:

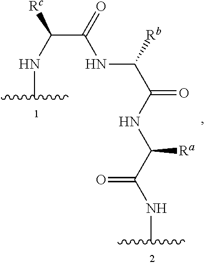

a four amino acid sequence having the formula:

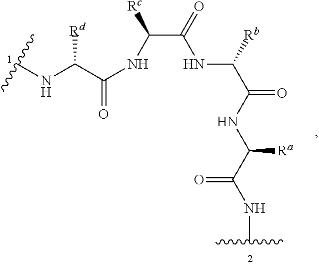

or
a five amino acid sequence having the formula:

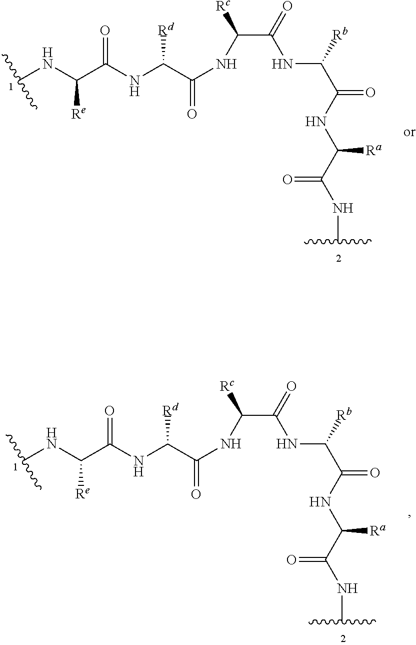

wherein wavy line 1, wavy line 2. R^{a}, R^{b}, R^{c}, R^{d}, and R^{e} are as defined above.

In some embodiments, Xⁿ has the formula:

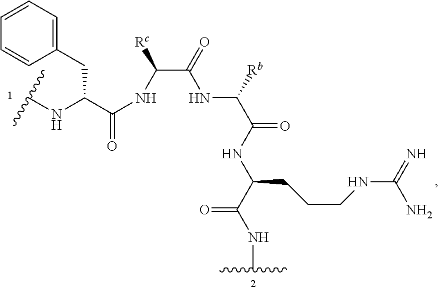

wherein wavy line 1, wavy line 2, R^{c} and R^{d} are as defined above.

In certain embodiments, the compound of Formula 1 has the formula:

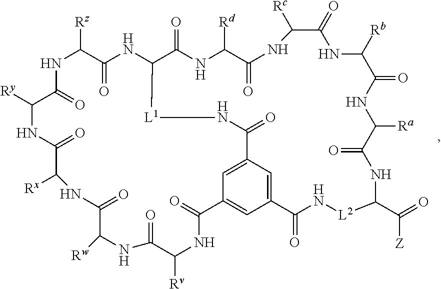

wherein Z, L¹, L², R^{a}, R^{b}, R^{c}, R^{d}, R^{v}, R^{w}, R^{x}, R^{y}, and R^{z} are as defined above. In some preferred embodiments, L¹ is n-butylene.

In some embodiments, the compound of Formula 1 has the formula:

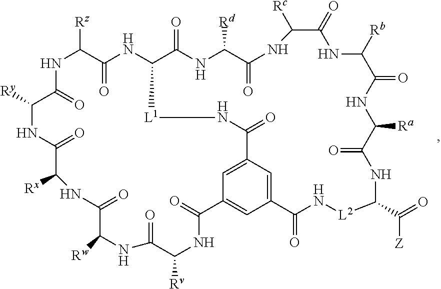

wherein Z, L¹, R^{a}, R^{b}, R^{c}, R^{d}, R^{v}, R^{w}, R^{x}, R^{y}, and R^{z} are as defined above. In some preferred embodiments, L¹ is n-butylene.

In some embodiments, the compound of Formula 1 has the formula:

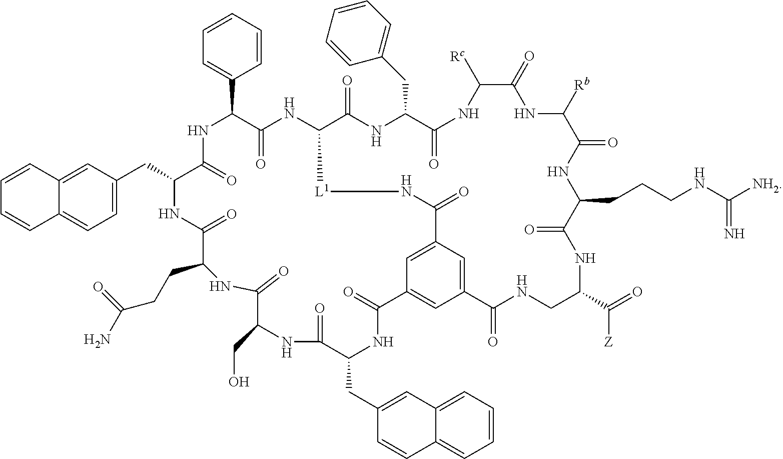

wherein Z, L¹, R^{b}, and R^{c} are as defined above. In some preferred embodiments, L¹ is n-butylene.

In some examples the compound of Formula 1 has the formula:

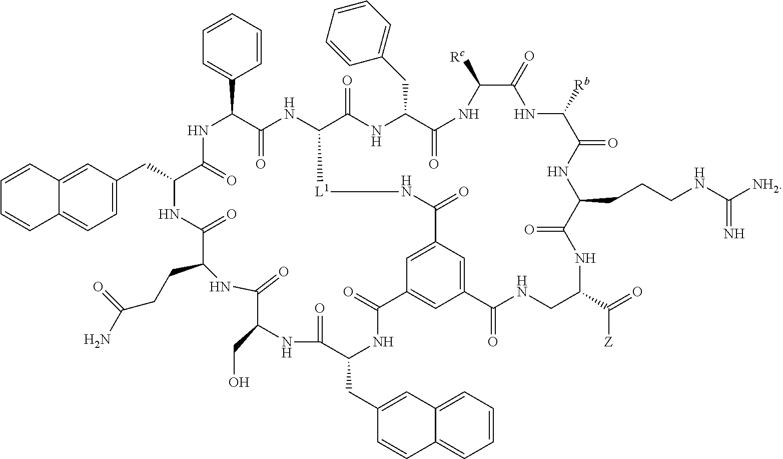

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl).

In the solid phase peptide synthesis method, the α—C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α—C-terminal carboxy peptides is 4-hydroxymethylphenoxymethyl-copoly (styrene-1% divinylbenzene) or 4-(2′,4′-dimethoxyphenyl-Fmoc-aminomethyl) phenoxyacetamidoethyl resin available from Applied Biosystems (Foster City, Calif.). The α—C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCI), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl) phenoxy-acetamidoethyl resin, the Fmoc group is cleaved with a secondary amine, preferably piperidine, prior to coupling with the α—C-terminal amino acid as described above. One method for coupling to the deprotected 4 (2′,4′-dimethoxyphenyl-Fmoc-aminomethyl) phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thioanisole, water, ethanedithiol and trifluoroacetic acid. In cases wherein the α—C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein.

Also provided herein are methods of treating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of killing a tumor cell in a subject. The method includes contacting the tumor cell with an effective amount of a compound or composition as described herein, and optionally includes the step of irradiating the tumor cell with an effective amount of ionizing radiation. Additionally, methods of radiotherapy of tumors are provided herein. The methods include contacting the tumor cell with an effective amount of a compound or composition as described herein, and irradiating the tumor with an effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. An effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane: sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### Examples

The following examples are for the purpose of illustration of the invention only and are not intended to limit the scope of the present invention in any manner whatsoever.

Materials. Reagents for peptide synthesis were purchased from Chem-Impex (Wood Dale, IL), NovaBiochem (La Jolla, CA), or Anaspec (San Jose, CA). Rink amide resin (100-200 mesh, 0.43 mmol/g) was from Chem-Impex (Wood Dale, IL). All solvents and other chemical reagents were obtained from Sigma-Aldrich, Fisher Scientific (Pittsburgh, PA), or VWR (West Chester, PA) and were used without further purification unless noted otherwise. Dynabeads M-280 streptavidin were purchased from Invitrogen (Carlsbad, CA). Cell culture media, fetal bovine serum (FBS), penicillin-streptomycin, 0.25% trypsin-EDTA, DPBS, 100× non-essential amino acids, sodium pyruvate solution, isopropyl β-D-1-thiogalactopyranoside (IPTG), guanosine 5′-[β,γ-imido]triphosphate (GppNHp), guanosine 5′-O-(3-thiotriphosphate) (GTPγS), guanosine 5′-diphosphate (GDP), 2,2,2-trifluoroethanol (TFE), streptavidin-alkaline phosphatase (SA-AP), protease inhibitor cocktail tablets, ampicillin, 5 (6)-carboxyfluorescein, and 5 (6)-carboxytetramethylrhodamine were purchased from Sigma-Aldrich (St. Louis, MO). Biotin-(PEG) 4-NHS ester, His-Pur™ Cobalt Resin, RIPA lysis buffer, CyQUANT™ LDH cytotoxicity assay, Lipofectamine 2000, and Alexa Fluor® 488 Annexin V/Dead Cell apoptosis kit were purchased from Thermo Fisher Scientific. Micro Bio-Spin™ 6 desalting columns were purchased from Bio-Rad (Hercules, CA). Anti-GST-Tb cryptate and anti-HA-d2 monoclonal antibodies were purchased from CisBio (Bedford, MA). The cell proliferation kit (MTT) was purchased from Roche (Indianapolis, IN). The Cell-Titer Glo® reagent was purchased from Promega (Madison, WI).

H358, NIH 3T3, A549, DLD-1, H1915 and HeLa cell lines were purchased from American Type Culture Collection (Manassas, VA). HEK293T cell line was a generous gift from Dr. Kotaro Nakanishi's group (The Ohio State University). H1299 cell line was a generous gift from Dr. Qi-En Wang's group (The Ohio State University). MDCK cell line stably expressing GFP-KRasG12V was a generous gift from Profs. John F. Hancock and Yong Zhou (University of Texas, Houston, TX). All cell lines were maintained in a humidified chamber with 5% CO₂ at 37° C. Other antibodies used are listed in the table below.

| Target Protein | Company | Catalog No. |
|---|---|---|
| GAPDH | Cell Signaling Technology | 5174 |
| Phospho-Akt (T308) | Cell Signaling Technology | 9275 |
| Phospho-Akt (S473) | Cell Signaling Technology | 9271 |
| Phospho-MEK1/2 | Cell Signaling Technology | 9121 |
| (Ser217/221) | | |
| Akt | Cell Signaling Technology | 9272 |
| MEK1/2 | Cell Signaling Technology | 9122 |
| Anti-rabbit IRDye® | LI-COR | 926-32211 |
| 800CW | | |
| Anti-mouse IRDye® | LI-COR | 926-32210 |
| 800CW | | |

Protein Expression and Purification. His₆-KRasG12V (1-186)-HA was expressed in Escherichia coli BL21 (DE3) cells in 2 L of LB containing 50 μg/mL kanamycin sulfate. The cells were grown in a shaker at 37° C. to an OD600 of 0.6 and protein expression was induced by the addition of 0.3 mM IPTG at 30° C. for 5 h. The cells were pelleted by centrifugation at 5000 rpm, and the cell pellets were stored at −80° C. For lysis, the cell pellet was thawed at RT and re-suspended in 50 mL of lysis buffer (50 mM Tris pH 7.5, 300 mM NaCl, 10 mM imidazole, 5 mM MgCl₂, 5 mM β-mercaptoethanol, 2 cOmplete™ protease inhibitor tablets (Roche), and lysozyme (100 μg/mL)) and stirred at 4° C. for 10 min. Protamine sulfate (250 mg) was added to precipitate nucleic acids, and lysis solution was stirred at 4° C. for 20 min. For complete lysis, the mixture was then sonicated at 70% amplitude on ice for 1 min (2-s pulses with an 8-s pause in between to maintain low temperature). The lysed solution was centrifuged at 15000 rpm for 30 min yielding a clear supernatant which was directly loaded onto 2 mL of pre-equilibrated His-Pur™ Cobalt resin (ThermoFisher) and incubated at 4° C. for 1 h to ensure complete binding. Pre-equilibration of the resin was performed by washing with 50 mM Tris pH 7.5, 300 mM NaCl, 10 mM imidazole, 5 mM MgCl₂, and 5 mM β-mercaptoethanol. The flow-through was discarded, and the resin was washed with 50 mL of 50 mM Tris, pH 7.5, 300 mM NaCl, 20 mM imidazole, 5 mM MgCl₂, and 5 mM β-mercaptoethanol and 100 mL of the same buffer with 30 mM imidazole. Elution was performed by adding 15 mL of 50 mM HEPES, pH 7.4, 150 mM NaCl, 150 mM imidazole, 2 mM β-mercaptoethanol to the resin and collecting the eluted protein by gravity filtration after 5 min of stationary incubation. After elution the protein was dialyzed into HTRF assay buffer (50 mM HEPES, pH 7.4, 150 mM NaCl, 5 mM MgCl₂, and 2 mM β-mercaptoethanol) using Amicon Ultra-15 centrifugal filter units (MWCO: 10 kDa) and concentrated to approximately 5 mg/mL. The concentration of the protein was determined using the Bradford assay (Bio-Rad) and the protein was aliquoted and frozen at −80° C. after the addition of 20% glycerol.

GST-RBD was expressed in E. coli BL21 (DE3) cells in 2 L of LB containing 50 μg/mL ampicillin. The cells were grown in a shaker at 37° C. to an OD600 of 0.6 and protein expression was induced by the addition of 0.1 mM IPTG at 30° C. for 5 h. The cells were pelleted by centrifugation at 5000 rpm, and the cell pellets were stored at −80° C. The cell pellet was thawed at RT and re-suspended in 50 mL lysis buffer (1× PBS buffer, pH 7.4, 137 mM NaCl, 2.7 mM KCl, 10 mM Na₂HPO₄) supplemented with 5 mM β-mercaptoethanol, 5 mM EDTA, 0.1% Triton-X-100, 200 μg/mL lysozyme, and 2 cOmplete™ protease inhibitor tablets (Roche). This mixture was stirred at 4° C. for 30 min and sonicated at 70% amplitude on ice for 2 min (in 2-s pulses with 8-s pauses in between to maintain low temperature). After sonication, 500 mg of protamine sulfate was added to the lysate and the solution was stirred for an additional 15 min at 4° C. The lysate was centrifuged at 15000 rpm at 4° C. for 30 min to yield a clear supernatant, which was directly loaded onto 2 mL of pre-equilibrated glutathione-Sepharose 4B resin (GE Healthcare) and incubated at 4° C. for 1 h to ensure complete binding. Pre-equilibration of the resin was performed by washing with 1× PBS buffer (pH 7.4) supplemented with 5 mM β-mercaptoethanol and 5 mM EDTA. The flow-through was discarded and the resin was washed with 100 mL of 1× PBS buffer, pH 7.4 supplemented with 5 mM β-mercaptoethanol, 5 mM EDTA and 0.5% Triton-X-100 and 100 mL of the same buffer without Triton-X-100. The bound protein was eluted from the column with 10 mM glutathione in 1×PBS, pH 7.4 containing 5 mM β-mercaptoethanol, dialyzed into 1×PBS, pH 7.4, 5 mM β-mercaptoethanol using Amicon Ultra-15 centrifugal filter units (MWCO: 10 kDa) and concentrated to approximately 10 mg/mL. The concentration of the protein was determined using the Bradford assay (Bio-Rad) and the protein was aliquoted and frozen at −80° C. after the addition of 20% glycerol.

Protein Biotinylation. To biotinylate His₆-KRasG12V (1-186)-HA, the freshly thawed protein solution (˜200 μM) was incubated with 3 eq. of biotin-(PEG) 4-NHS (20 mM stock) for 2 h on ice. The mixture was desalted using Micro Bio-Spin™ 6 Desalting Columns (Bio-Rad, USA, MWCO: 6 kDa) to remove any excess biotin. The protein concentration was re-measured using Bradford reagent and the protein was stored at −80° C. after the addition of 20% glycerol.

Nucleotide Exchange of Ras Protein. Nucleotide exchange was performed by incubating purified KRas (˜100 μM) with 200 eq. EDTA and 40 eq. GppNHp, GTPγS, or GDP for 1 h on ice. 34 After 1 h, the reaction was quenched by the addition of 800 eq. of MgCl₂ and incubating the solution for 15 min. This mixture was desalted using Micro Bio-Spin™ 6 Desalting Columns (Bio-Rad, USA, MWCO: 6 kDa) preequilibrated with the proper assay buffer. Protein concentration was re-measured using Bradford reagent and the protein was stored at −80° C. after the addition of 20% glycerol.

Peptide synthesis. Bicyclic peptides were synthesized manually on Rink Amide resin (50-100 mesh, 0.43 mmol/g, Chem-Impex). Typical Fmoc deprotection was performed using 20% piperidine in DMF (v/v) twice for 5 min at RT while mixing. The typical coupling reaction involved 5 eq. of Fmoc-amino acid, 5 eq. HATU and 10 eq. diisopropylethylamine (DIPEA) in DMF for 30 min at RT while mixing. Coupling of L-phenylglycine (Phg) contained 3 eq. of Fmoc-L-Phg, 3 eq. of 1-COMU and 4 eq. of 2,6-lutidine in DMF and was repeated twice (each 30 min incubation). Following Phg coupling, the resin was treated with 15 eq. of acetic anhydride and 15 eq. of DIPEA in DCM for 30 min at RT to acetylate any unreacted amine and the Fmoc group was removed with 10% piperidine (2×5 min). After all desired residues were coupled and the N-terminal Fmoc group was removed, the peptide was treated (3×) with 10 eq. of trimesic acid, 3 eq. of HATU, and 15 eq. of DIPEA in DMF for 1 h. The allyl protecting group was removed by treatment in the dark with 0.3 eq. of Pd(PPh₃)₄ and 10 eq. of phenylsilane in dry DCM (3×15 min). The resin was incubated with 1 M HOBt in DMF for 30 min and the peptide was cyclized by using 10 eq. of PyBOP, 10 eq. of HOBt, and 20 eq. of DIPEA for 1 h at RT. Cleavage and deprotection of the bicyclic peptide was performed by incubating the resin with 92.5/2.5/2.5/2.5 (v/v) TFA/TIPS/DMB/H₂O for 3 h at RT. The crude peptide was triturated with cold ethyl ether (3×) and purified by reversed-phase HPLC equipped with a semipreparative C18 column. The purity of all peptides used in this work was confirmed to be ≥95% by analytical HPLC (monitored at 214 nm) and their authenticity was confirmed by high-resolution MALDI-TOF mass spectrometry.

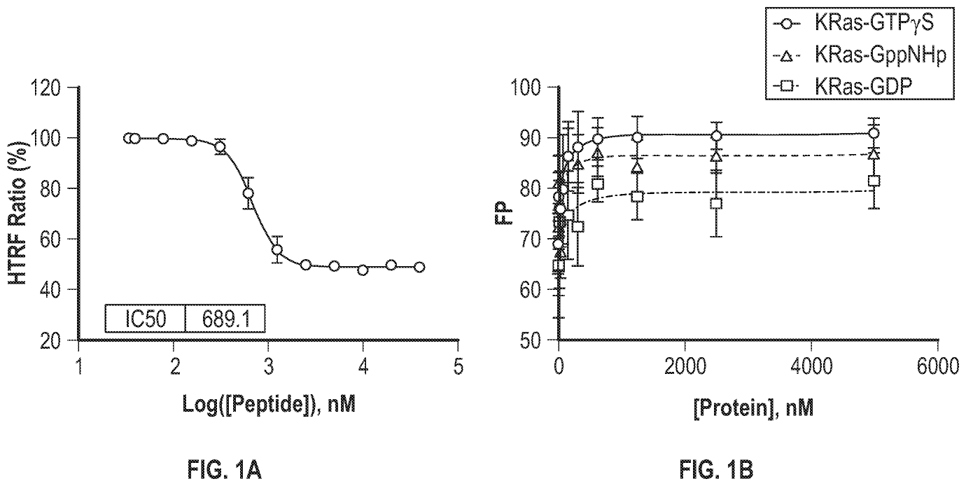

B4-27 Is a Selective Inhibitor of Ras-GTP. B4-27 was labeled with a fluorescein at its C-terminus through a (miniPEG) 2-Lys linker (B4-27^{FAM}) and tested for binding to GTPγS-, GppNHp-, or GDP-loaded KRasG12V by fluorescence polarization (FP). B4-27^{FAM} bound KRas-GTPγS, KRas-GppNHp, and KRas-GDP with Kp values of 42, 21, and 227 nM, respectively (FIG. 1B). Compared to B3, which has Kp values of 1.6 and 9.3 M for KRas-GppNHp and KRas-GDP, respectively, B4-27 represents a 76-fold improvement in Ras binding potency but retains the ˜ 10-fold selectivity for the active vs inactive form of KRas. B4-27^{FAM} was tested for binding to five arbitrarily selected control proteins, including bovine serum albumin (BSA), GST-RBD, purine nucleoside phosphorylase (PNP), protein-tyrosine phosphatase 1B (PTP1B), and mouse double minute 2 homolog (MDM2). B4-27^{FAM} showed weak binding to BSA (KD)=57 μM) but no appreciable binding to GST-RBD, PNP, PTP1B, or MDM2. B4-27 is therefore a selective ligand of the active form of KRas.

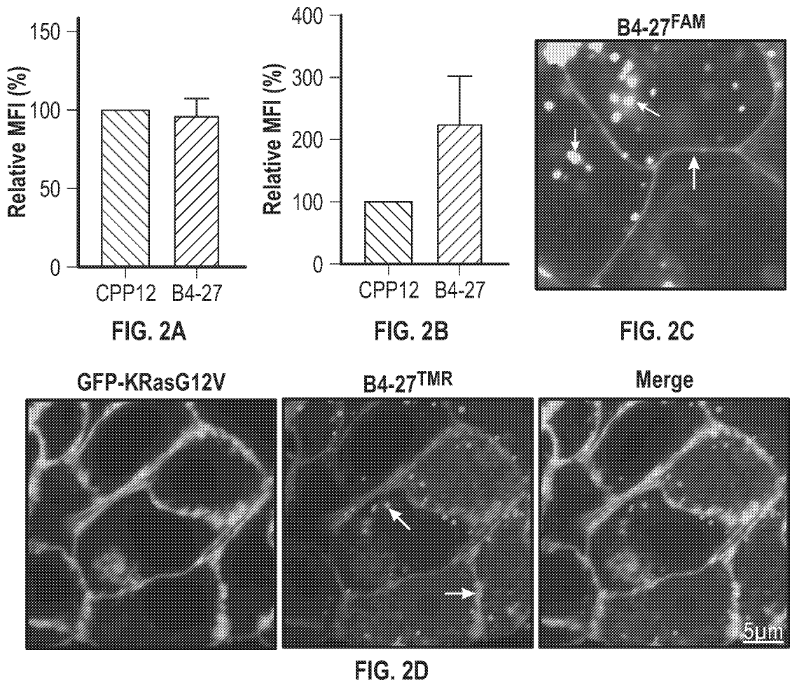

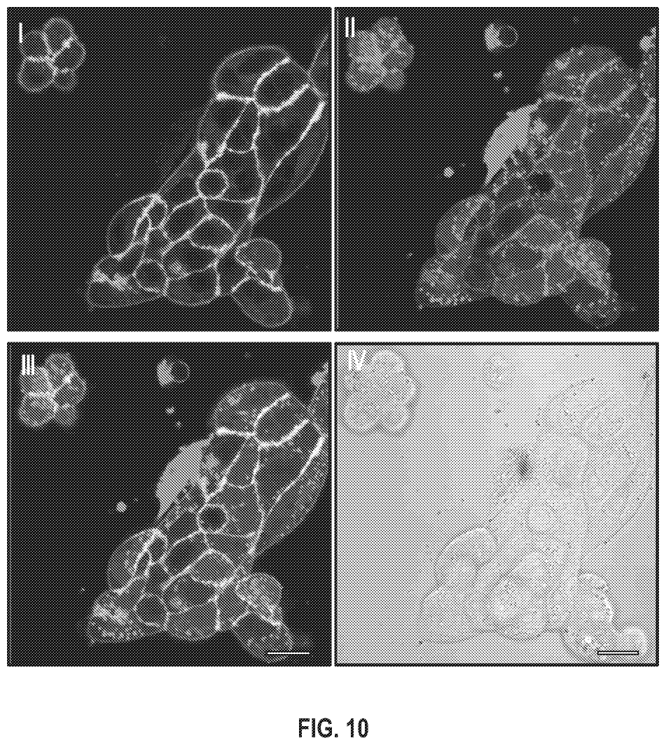

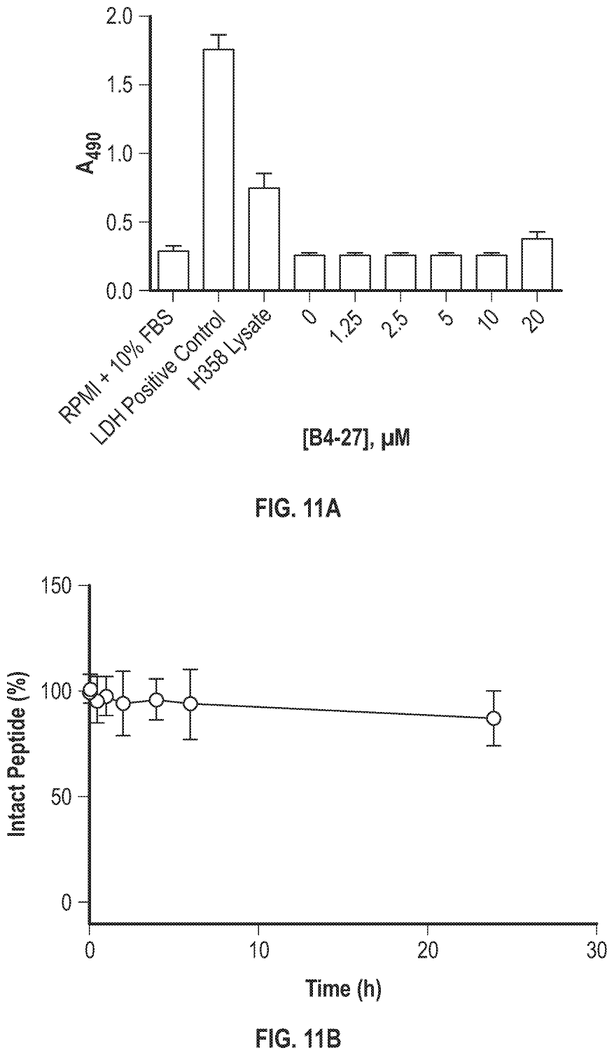

B4-27 Efficiently Enters the Mammalian Cell and Co-localizes with KRas. The cellular entry efficiency of B4-27 was assessed by flow cytometry analysis of HeLa cells treated with B4-27^{FAM} and compared to that of CPP12, one of the most active cyclic cell-penetrating peptides. Cells treated with 1 or 5 μM B4-27^{FAM} showed mean fluorescence intensity (MFI) values of 97 and 226%, respectively, relative to that of CPP12 (100%) (FIGS. 2A, 2B). Under similar conditions, CPP12 has a cytosolic entry efficiency of 120% (defined as the ratio of cytosolic over extracellular concentration). To determine the intracellular distribution of B4-27, HeLa, H358, and A549 cells were examined after treatment with 5 μM B4-27^{FAM} for 2 h by live-cell confocal microscopy. All cells became intensely fluorescent, exhibiting a mixture of diffuse fluorescence throughout the entire cell volume and highly fluorescent puncta in the cytoplasmic region (FIG. 2C, 8A-8C). The presence of diffuse fluorescence indicates that a significant fraction of the internalized B4-27^{FAM} had reached the cytosol, while the fluorescence puncta likely represent the B4-27^{FAM} molecules that had not yet escaped the endosomes. In H358 and A549 cells, a fraction of B4-27^{FAM} molecules was concentrated at the plasma membrane (FIG. 2C), suggesting that it bound to the membrane-associated Ras population. To test this notion, we treated Madin-Darby canine kidney (MDCK) cells stably expressing a green fluorescent protein (GFP)-KRasG12V fusion with 5 μM tetramethylrhodamine-labeled B4-27 (B4-27^{TMR}) and imaged the cells by live-cell confocal microscopy. The plasma membrane-bound B4-27^{TMR} co-localized with the GFP-KRasG12V fusion protein, especially at the interfaces between adjacent cells (FIG. 2d). Interestingly, for plasma membrane regions that are not in direct contact with neighboring cells, there is much less accumulation of GFP-KRasG12V or B4-27^{TMR} (FIG. 10). These observations demonstrate that that B4-27 efficiently enters mammalian cells and a fraction of the inhibitors are bound to Ras proteins at the plasma membrane.

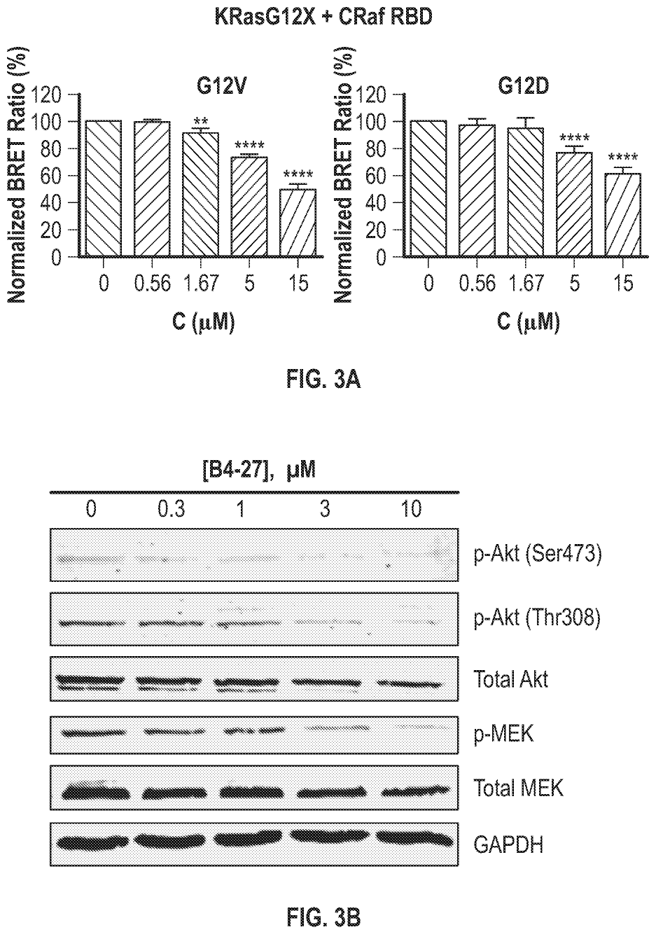

B4-27 Inhibits Intracellular Ras-Effector Interaction and Ras Signaling Pathways. We tested the ability of B4-27 to inhibit the intracellular Ras-effector interactions by using a Ras-Raf BRET2 assay. Human embryonic kidney (HEK293T) cells were transfected with plasmids expressing a Renilla luciferase variant-KRasG12V (or G12D) fusion protein (RLuc8-KRas) as the BRET donor (fusion protein) and a GFP-CRAF Ras-binding domain (RBD) fusion protein as the acceptor. Under normal conditions, interaction between RLuc8-KRas and GFP-CRAF RBD produces a BRET signal in the presence of a luciferase substrate, Coelenterazine 400a; on the other hand, inhibition of the Ras-Raf interaction would decrease the BRET signal. Treatment of HEK293T expressing RLuc8-KRasG12V (or RLuc8-KRasG12D) and GFP-CRAF RBD with B4-27 (0-15 μM) significantly and dose-dependently reduced the BRET signal (FIG. 3A), indicating that B4-27 is capable of blocking KRasG12V and KRasG12D from binding to its downstream effector proteins in a cellular context.

We next examined the ability of B4-27 to inhibit the Ras/Raf/MEK/ERK and Ras/PI3K/Akt signaling pathways. H358 cells were treated with varying concentrations of B4-27 (0-10 μM) for 4 h and stimulated for 10 min with epidermal growth factor (EGF; 50 ng/mL). The cells were lysed and the phosphorylation levels of MEK and Akt were monitored by Western blot analysis using antibodies specific for phosphorylated Akt [anti-p-Akt (Ser473) and anti-p-Akt (Thr308)] and MEK (anti-p-MEK). B4-27 dose-dependently inhibited the phosphorylation of Akt and MEK (IC₅₀≈3 μM), while the total Akt and MEK levels remained relatively constant (FIG. 3B). Thus, B4-27 effectively inhibits the signaling pathways downstream of Ras.

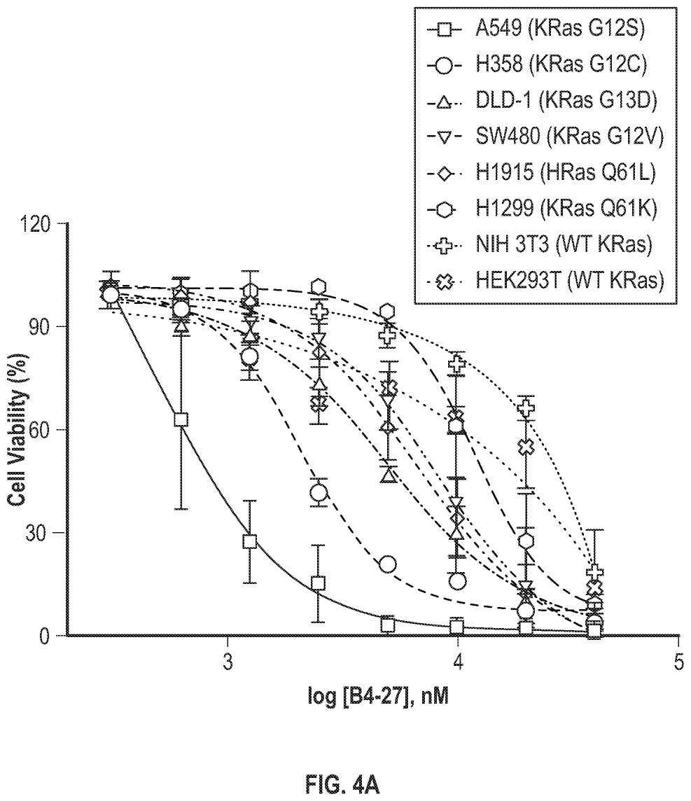

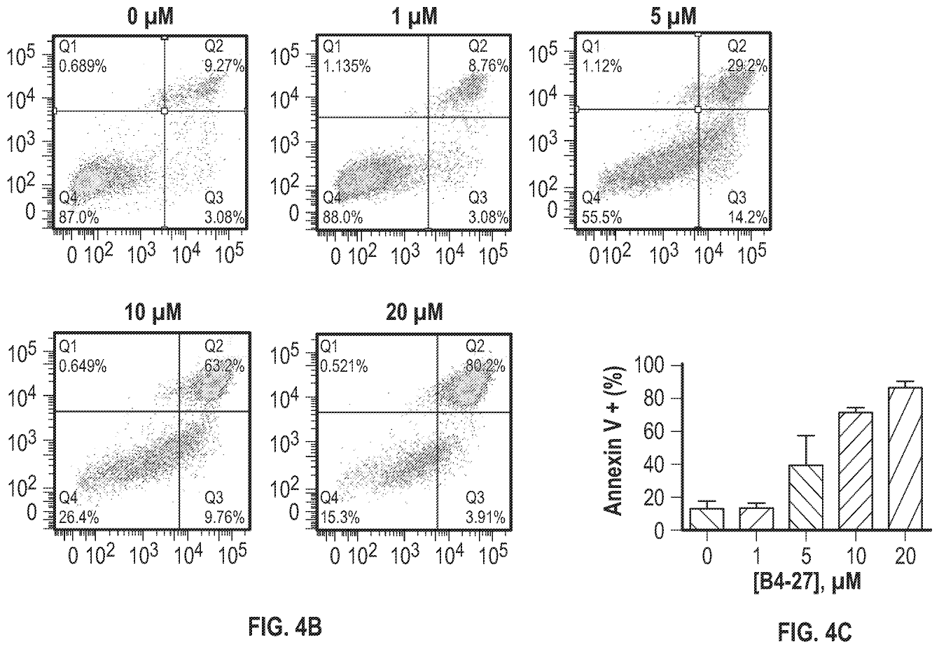

B4-27 Is a Pan-Ras Inhibitor and Induces Apoptosis of Ras Mutant Cancer Cells. Given its ability to block the Ras-Raf interaction (and likely Ras-PI3K interaction as well), B4-27 likely binds at or near the effector-binding site on Ras. Because the effector-binding site is the same for all 4 Ras isoforms, we expected B4-27 to be a pan-Ras inhibitor, capable of inhibiting all 4 Ras isoforms, regardless of the mutational status (i.e., WT and G12, G13, and Q61 mutants). To test this notion, we tested B4-27 against a panel of human cancer cell lines carrying different Ras mutations, including non-small cell lung cancer cell lines (H358, heterozygous KRas^{G12C} and A549, homozygous KRas^{G12S}), colorectal cancer cell lines (DLD-1, heterozygous KRas^{G13D} and SW480, homozygous KRas^{G12V}), a lung cancer cell line carrying mutant HRas (H1915, HRas^{Q61L}), and a lung cancer cell line carrying mutant NRas (H1299, NRas^{Q61K}). B4-27 reduced the viability of all tested Ras mutant cancer cell lines with EC₅₀ values of 0.5±0.3 μM for A549 cells, 2.1±0.2 μM for H358 cells, 4.9±0.7 μM for DLD-1 cells, 8.7±3.4 UM for SW480 cells, 6.8±2.2 μM for H1915 cells, and 11.9±2.3 μM for H1299 cells (FIG. 4A). We also tested B4-27 against two non-cancerous cell lines that carry WT Ras, including a murine fibroblast cell line (NIH 3T3) and a human embryonic kidney (HEK293T) cell line. B4-27 reduced the viability of the non-cancerous cell lines, but only at high concentrations (EC₅₀≥20 μM). Several factors may contribute to the greater sensitivity of Ras mutant cancer cells to B4-27, including more robust endocytic uptake of the inhibitor, reduced binding affinity of Ras mutants to effector proteins, and “addiction” to Ras activity for signaling/survival by cancer cells.

Since simultaneous inhibition of the MEK/ERK and PI3K/Akt signaling pathways is known to cause apoptosis of cancer cells, B4-27 is expected to induce apoptosis in Ras mutant cancer cells. To test this possibility, H358 cells were treated with varying concentrations of B4-27 for 4 h, stained with Alexa Fluor® 488-annexin V and propidium iodide (PI), and analyzed by flow cytometry. The presence of a significant population of annexin V-positive but PI-negative cells at intermediate B4-27 concentrations (5 and 10 μM) and the absence of annexin V-negative and PI-positive cells are consistent cell death by apoptotic mechanisms (FIG. 4B). At 10 μM B4-27, >70% of the cell population had already undergone or was undergoing apoptosis (FIG. 4B, 4C). To determine whether B4-27 caused any cell death by nonspecific mechanisms, e.g., membrane disruption, we examined the cell culture medium of H358 cells after B4-27 treatment for the presence of lactate dehydrogenase (LDH) activity. B4-27 did not cause significant LDH release until concentrations at ≥20 μM (FIGS. 9A-9C). Taken together, our results strongly suggest that B4-27 efficiently enters the cytosol of mammalian cells, binds selectively to WT and mutant Ras proteins, blocks Ras proteins from engaging their downstream effector proteins, and induces apoptosis of Ras mutant cancer cells.

B4-27 Is Metabolically Stable. The proteolytic stability of B4-27 was assessed by incubating it in human serum at 37° C. for up to 24 h and analyzing the reaction mixture by analytical HPLC. Approximately 85% of the B4-27 remained intact after 24 h of incubation, indicating a serum t_{1/2} of >24 h (FIGS. 9A-9C).

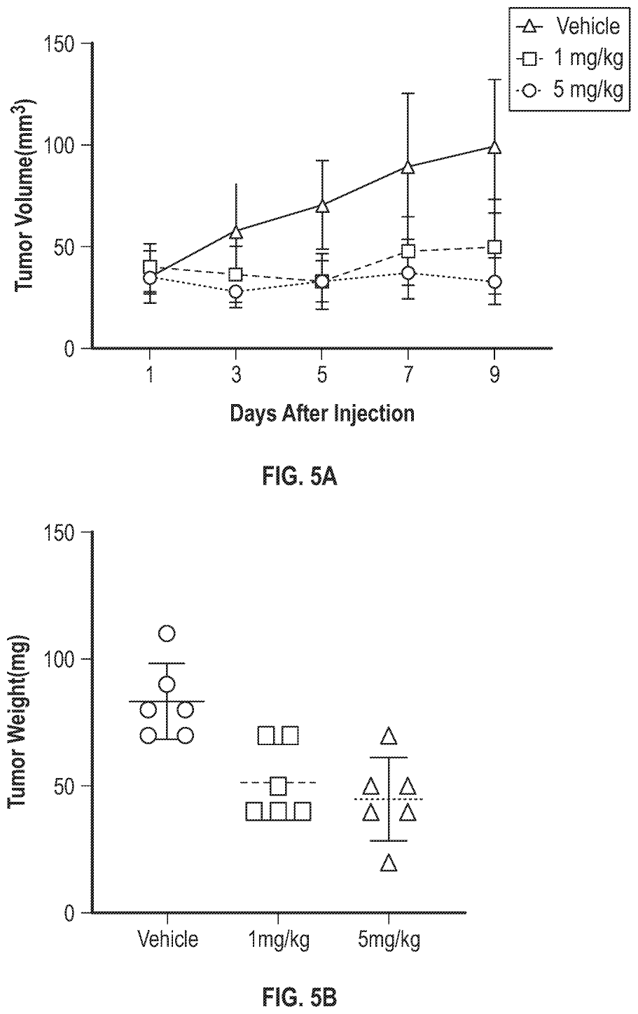

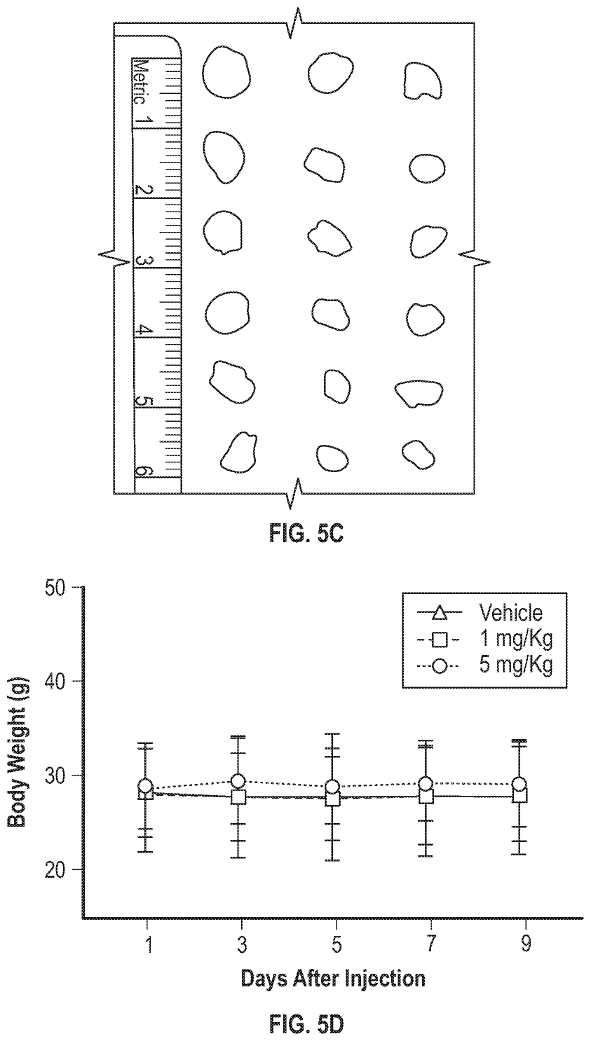

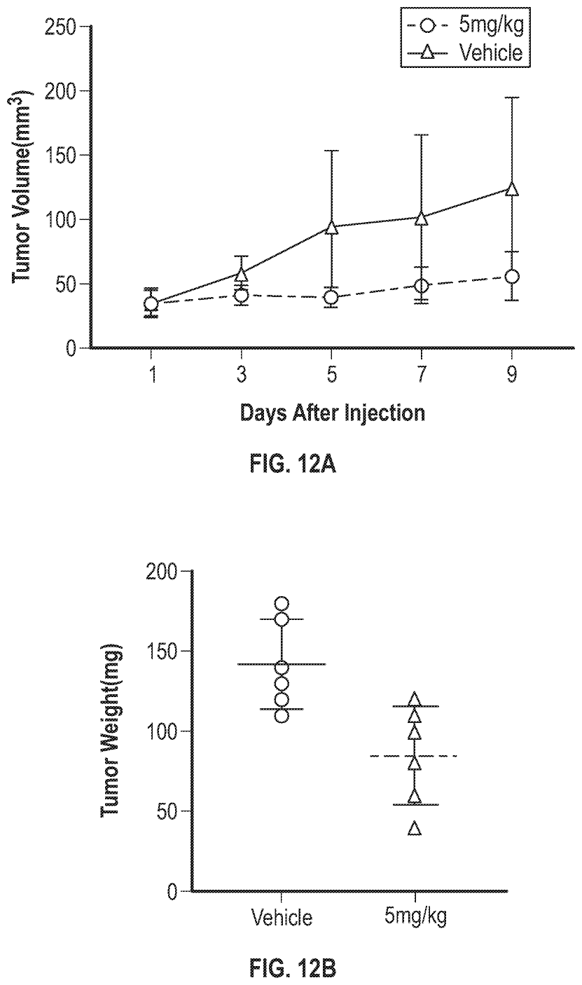

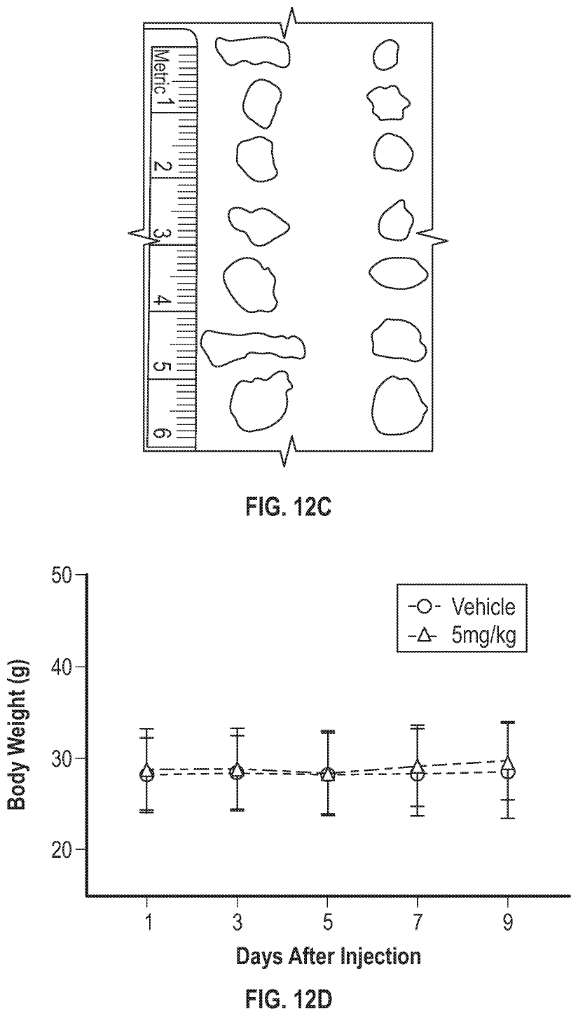

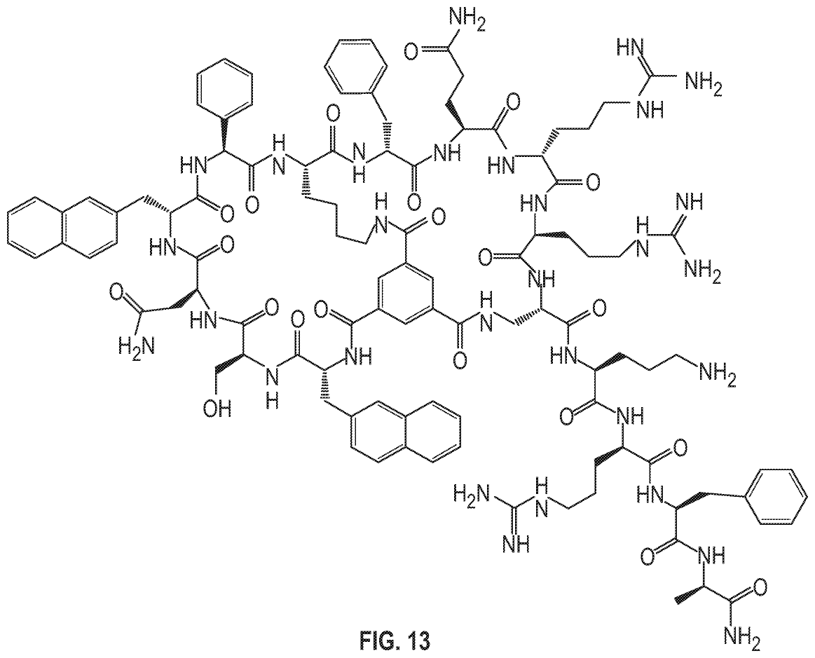

B4-27 Inhibits Tumor Growth in Mouse Xenograft Models. To test whether B4-27 can suppress tumor growth in vivo, we generated an A549 xenograft model. Approximately 1.5 million A549 cells were subcutaneously injected into nude mice. When the tumors grew to ˜40 mm³ in volume, the mice (n=6 in each group) were dosed with B4-27 at 1 or 5 mg/kg per day or vehicle (PBS) through tail vein injection for a total of 9 days. B4-27 significantly suppressed tumor growth at both tested concentrations, with an average tumor volume of ˜50 mm³ for the 1 mg/mL group and ˜35 mm³ for the 5 mg/mL group, while the vehicle-treated group had an average tumor volume of ˜ 100 mm³ on day 9 after injection (FIG. 5A). Importantly, the 5-mg/kg treatment completely halted tumor growth during the 9-day period. The average tumor weight on the day of the harvest was also 39% and 46% less for the 1- and 5-mg/kg treatment groups, respectively, than that of the control group (FIG. 5B, 5C). B4-27 treatment did not result in any significant body weight loss and did not induce any apparent toxicity (FIG. 5D). B4-27 was also tested in an H358 xenograft model and a daily dose of 5 mg/kg largely suppressed the tumor growth after 9 days (FIGS. 12A-12C).

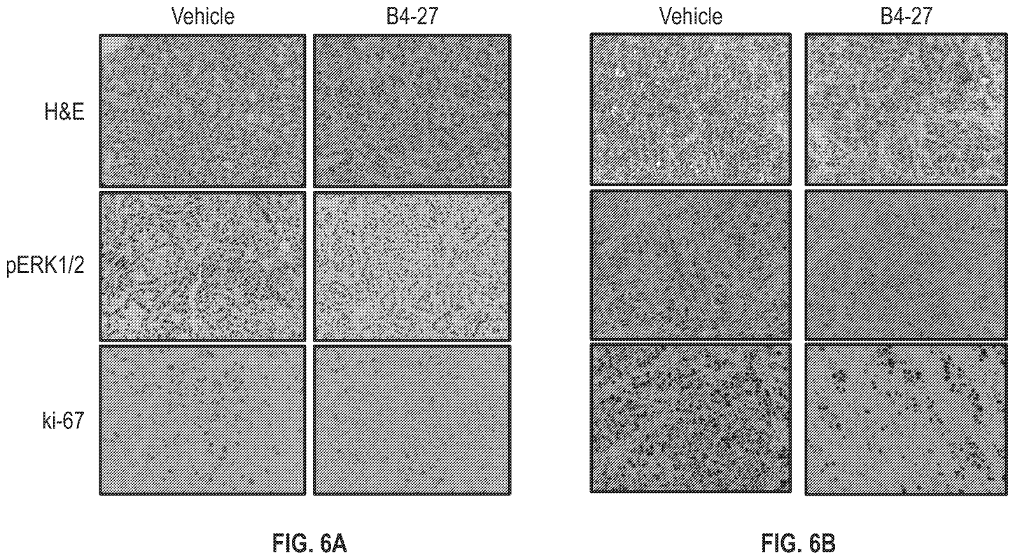

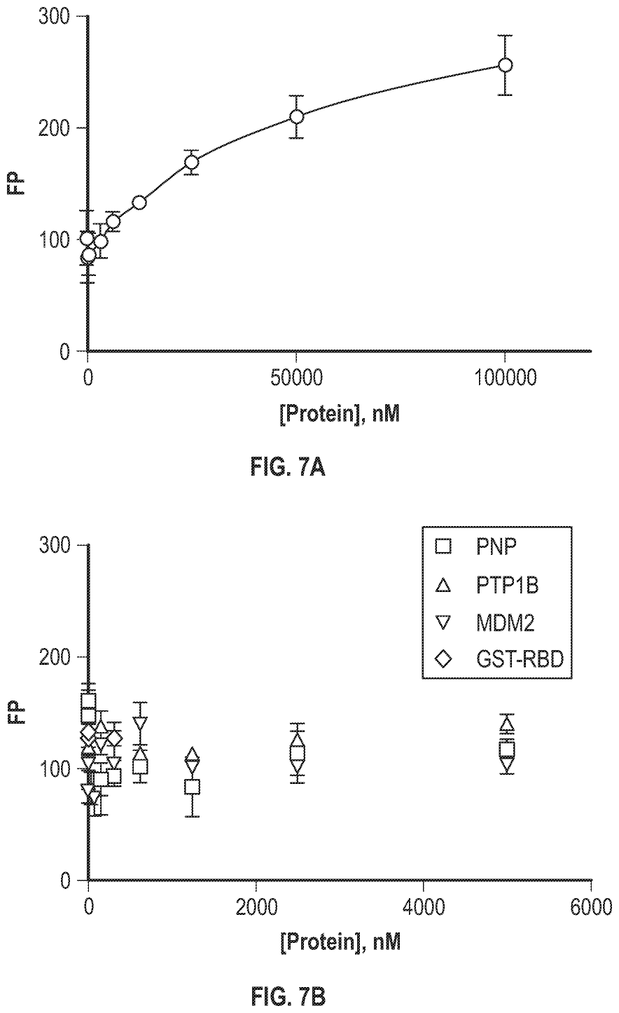

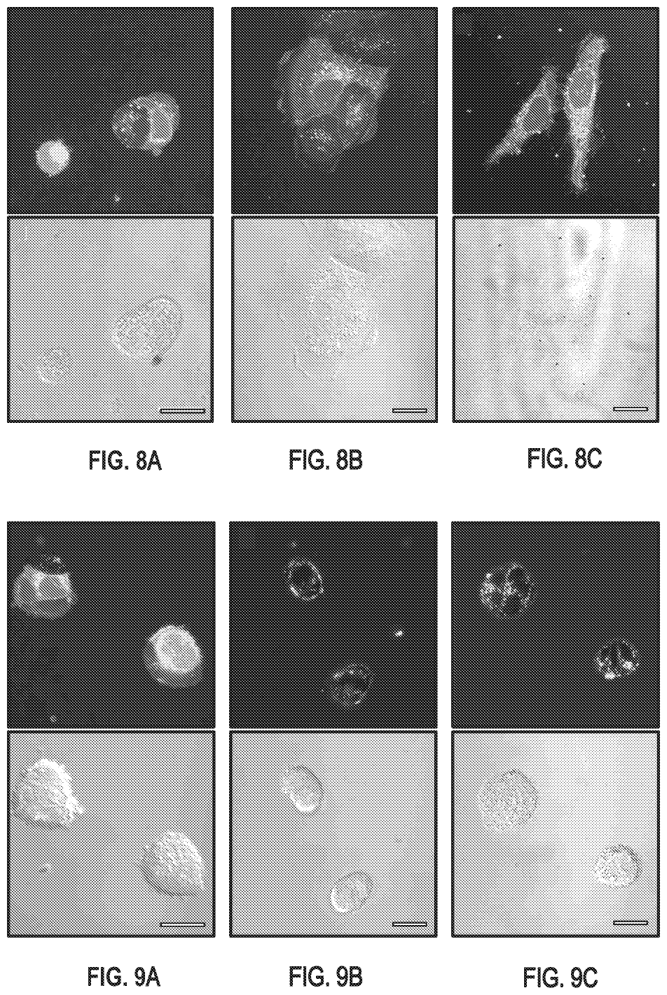

Representative samples of tumors were analyzed by immunohistochemistry (IHC) using hematoxylin and eosin (H&E), pERK, and Ki-67 staining. Immunohistochemical analysis models (FIGS. 6A, 6B), confirming that tumor growth inhibition was caused by suppression of Ras-mediated cell signaling. In addition, IHC revealed a large decrease in cell proliferation as indicated by Ki-67 staining.

Fluorescent Labeling of Peptides. To prepare fluorescently labeled B4-27, Fmoc-Lys (Mtt)—OH and two Fmoc-miniPEG residues were added at its C-terminus during peptide synthesis. After the bicyclic peptide synthesis was complete (while peptide still on resin), the 4-methyltrityl (Mtt) group on the C-terminal Lys was removed by treatment with 2% TFA and 1% triispopropylsilane in DCM (6×5 min). The peptide was incubated overnight with 2 eq. of 5 (6)-carboxyfluorescein or 5 (6)-carboxytetramethylrhodamine, 5 eq. of PyBOP, 5 eq. of HOBT, and eq. of DIPEA in DMF at RT. Peptide cleavage, deprotection, purification, and quality assessment were performed as previously described.

HTRF Assay. Recombinant GST-RBD (50 nM), His₆-KRasG12V (1-186)-HA (50 nM), anti-HA monoclonal antibody labeled with d2 acceptor (CisBio, USA, 2 μg/mL), anti-GST monoclonal antibody labeled with Tb cryptate donor (CisBio, USA, 2.5 μg/mL), and varying concentrations of a peptide (0-40 μM) were mixed in HTRF assay buffer (Vₜₒₜₐₗ=20 μL) in a 384-well plate (Greiner). The plate was incubated for 1 h at RT and the HTRF signals (the donor/acceptor ratio) were measured on a Tecan Infinite M1000 Pro microplate reader using HTRF Terbium program and plotted as a function of the peptide concentration. The data was analyzed using GraphPad Prism 6.0 and IC₅₀ values were obtained by fitting the data to the dose-response inhibition curves.

Fluorescence Polarization. FP experiments were performed by incubating 100 nM B4-27^{FAM} with varying concentrations of KRas loaded with GTPγS, GDP, or GppNHp in HBS in the presence of 1% 2,2,2-trifluoroethanol (TFE). TFE stabilizes the Switch I region of HRas in a physiologically relevant conformation. The solution was incubated for 1 h at RT with gentle mixing and transferred into black 384-well microplates. Fluorescence polarization was measured on Tecan Infinite M1000 plate reader, and titration curves were fitted using GraphPad PRISM v.6 to the following equation:

$$F P = \frac{\begin{matrix}( {A}_{min} + ( {A}_{max} \times \frac{{Q}_{b}}{{Q}_{f}} - {A}_{min} ) \\ ( \frac{( L + x + {K}_{d} ) - \sqrt{( {( L + x + {K}_{d} )}^{2} - 4 L x )}}{2 L} ) )\end{matrix}}{\begin{matrix}( 1 + ( \frac{{Q}_{b}}{{Q}_{f}} - 1 ) \\ ( \frac{( L + x + {K}_{d} ) - \sqrt{( {( L + x + {K}_{d} )}^{2} - 4 L x )}}{2 L} ) )\end{matrix}}$$

where FP is the measured polarization, Amin is the minimum FP value, Amax is the maximum FP value, Q_{b} is the quantum yield of the bound fluorophore, Q_{f} is the quantum yield of the free fluorophore, L is the ligand concentration, Kp is the dissociation constant, and x is the protein concentration.

Cell Culture. NIH 3T3, HEK293T, HeLa, and MDCK-GFP-KRasG12V cells were cultured in Dulbecco's modified eagle's medium (DMEM) supplemented with 10% FBS and 1% penicillin-streptomycin sulfate. H358, H1299, H1915, and DLD-1 cells were cultured in RPMI-1640 medium supplemented with 10% FBS and 1% penicillin-streptomycin sulfate. A549 cells were cultured in F-12K medium supplemented with 10% FBS and 1% penicillin-streptomycin sulfate. Cells were cultured in a humidified incubator at 37° C. in the presence of 5% CO₂.

MTT Cell Viability Assay. H358 cells were seeded in a transparent 96-well plate at a density of 5,000 cells/well in 100 μL of full growth medium and grown overnight. Next day, cells were treated with varying concentrations of a serially diluted peptide (0-40 μM) in 10 μL of assay media containing 10% FBS and incubated at 37° C. with 5% CO₂ for 96 h. After incubation, 10 μL of MTT stock solution (Roche) was added to each well. After an additional 4 h incubation at 37° C., 100 μL of SDS-HCl solubilizing solution was added to each well and the plate incubated overnight at 37° C. The absorbance of the formazan product was measured at 570 nm on a Tecan M1000 plate reader.

Cell-Titer Glo® 2.0 Viability Assay. Cells (H358, DLD-1, A549, SW480, H1915, H1299, NIH 3T3, or HEK293T) were seeded in an opaque 96-well microplates plate at a density of 5,000 cells/well in 100 μL of full growth medium and grown overnight. Next day, cells were treated with varying concentrations of a serially diluted peptide (0-40 μM) in 10 μL of assay media containing 10% FBS and incubated at 37° C. with 5% CO₂ for 96 h. After incubation, the plate was removed and pre-equilibrated to RT before the addition of 100 μL of Cell-Titer Glo® 2.0 reagent (Promega, WI, USA) to each well. The plate was incubated for 15 min on a rotary shaker in the dark and the luminescence was detected on a Tecan Infinite M1000 Pro microplate reader.

Annexin-V/PI Cell Apoptosis Assay. H358 cells were seeded into a 12-well microplate (100,000 cells/well) in 1 mL of RPMI containing 10% FBS and 1% penicillin-streptomycin sulfate. The next day, the media was removed, and each well was washed twice with DPBS before the addition of peptide B4-27 at the desired concentration in 1 mL of RPMI media containing 10% FBS. After 4 h of treatment, the media was collected into a 15-mL falcon tube. Adherent cells were washed with DPBS and removed from each well by treating with 300 μL of 0.25% (w/v) trypsin for 5 min at 37° C. and added to the corresponding falcon tube. Following centrifugation and resuspension in DPBS (repeated twice) to remove any remaining trypsin, the pelleted cells were re-suspended in 100 μL of 1× annexin-binding buffer. Five μL of Alexa Fluor® 488 annexin V and 1 μL of propidium iodide (PI, 100 μg/ml) were added to the cell suspension. The tubes were incubated on a rotary shaker for 15 min at RT to allow for staining, and 400 μL of annexin-binding buffer was added to each sample. The stained cells were immediately analyzed on a BD LSR Fortessa flow cytometer, measuring the emission at both 530 nm and 575 nm.

Intracellular Ras-Raf BRET Assay. HEK293T cells were seeded into a 6-well microplate (650,000 cells/well) in 1 mL of DMEM containing 10% FBS and 1% penicillin-streptomycin sulfate. The next day, cells were transfected with an appropriate amount of BRET-based Ras biosensor using Lipofectamine 2000 transfection reagent. After 24 h of incubation, cells were re-seeded in a white 96-well plate in OptiMEM medium supplemented with 4% FBS. Cells were incubated for 4 h at 37° C. and then treated with peptide B4-27 at 0, 0.56, 1.67, 5, or 15 μM in OptiMEM+4% FBS (final 0.5% DMSO in each well) in quadruplicates. Cells were incubated for 20 h at 37° C. and Coelenterazine 400a (10 UM final concentration) was added to cells right before the measurement. BRET2 reading was measured on a Tecan M1000 plate reader using Dual Luminescence module.

Western Blot Analysis. H358 cells were maintained in RPMI-1640 supplemented with 10% FBS and 1% penicillin-streptomycin sulfate. A549 cells were maintained in DMEM supplemented with 10% FBS and 1% penicillin-streptomycin sulfate. Cells (1×10⁶ cells/well) were seeded in a 6-well plate overnight, washed with DPBS once, and treated with indicated concentrations of peptide B4-27 in full growth media for 4 h. DMSO was kept at 0.5% (v/v) in all wells. Before harvesting, cells were stimulated with EGF (50 ng/mL) for 10 min. The cells were washed twice with cold DPBS, detached by treatment with 0.25% Trypsin-EDTA solution (0.3 mL/well), and all fractions were collected. After centrifugation in a microcentrifuge (500 g, 5 min), cell pellets were lysed on ice for 30 min in IP lysis buffer (150 μL/sample) supplemented with protease and phosphatase inhibitors. Cell lysates were centrifuged at 15000 rpm in a microcentrifuge for 10 min, and the extracted proteins in the supernatant were collected. The total protein concentration was measured by using BCA Protein Assay Kit (Thermo, #23235), and equal amounts of protein were loaded onto different lanes of a 12% SDS-PAGE gel. After separation by electrophoresis, the proteins were transferred electrophoretically to a 0.45 μm nitrocellulose membrane at 4° C. The membrane was first blocked with 10% nonfat dry milk in TBST (20 mM Tris, pH 7.5, 150 mM NaCl, 0.1% (v/v) Tween-20) at 4° C. for 1 h, and then incubated with the proper primary antibody at 4° C. overnight. The antibody sources and conditions used were as follows: anti-MEK and anti-pMEK monoclonal antibodies (1:1000 dilution, Cell Signaling Technologies, 9122 and 9121), anti-Akt and anti-pAkt monoclonal antibodies (1:200 dilution, Cell Signaling Technologies, 9272, 9271 and 9275), anti-GAPDH monoclonal antibodies (1:1000 dilution, Cell Signaling Technologies, 5174). The membrane was washed three times with TBST and incubated with IRDye secondary antibodies (LI-COR, 1:10000 dilution) at RT for 2 h. The membrane was again washed with TBST three times, and fluorescent signals were recorded using a LICOR Odyssey CLx instrument.

Serum Stability Assay. Diluted human serum (25%; H4522 human serum, Sigma) was incubated at 37° C. for 15 min and added to peptide stock in DMSO to ˜100 μM final peptide concentration. The solution was incubated at 37° C. and 50-μL aliquots were withdrawn at various time points. This solution was mixed with 50 μL of 15% trichloroacetic acid (TCA) in MeOH and 50 μL of acetonitrile, and the mixture was stored at 4° C. overnight. Finally, the samples were centrifuged at 15,000 rpm for 10 min in a microcentrifuge, and the supernatant was analyzed by reversed-phase HPLC equipped with an analytical C18 column (Waters). The amount of peptide remaining at each time point was determined by integrating the area under the peptide peak in the resulting HPLC chromatogram (monitored at 214 nm) and comparing to the peptide amount at time zero.

LDH Release Assay. H358 cells were seeded into a transparent 96-well microplate (5,000 cells/well) in 100 μL of RPMI containing 10% FBS and 1% penicillin-streptomycin sulfate. The next day, cells were treated with varying concentrations of serially diluted peptide B4-27 (0-20 μM) in 10 μL of assay media containing 10% FBS. Ten μL of sterile H₂O was added to the media for the spontaneous LDH release control and 10 μL of 10× lysis buffer was added to the media for the maximum LDH release control. After 45 min of treatment at 37° C., 50 μL of each sample, 1× LDH positive control, and RPMI containing 10% FBS and 1% penicillin-streptomycin sulfate were transferred to a 96-well flat-bottom plate. Fifty μL of LDH assay solution was added to each well and the plate was incubated on a rotary shaker for 15 mins at RT in the dark. The LDH reaction was quenched by the addition of 50 μL of stop solution and the absorbances at 490 and 670 nm were measured on a Tecan M1000 plate reader. % LDH release was calculated according to the manufacturer's protocol.

Confocal Microscopy. HeLa and A549 cells were seeded in a 35-mm glass-bottomed microwell dish with 4 compartments (Greiner) at a density of 5×10⁴ cells/mL (300 μL in each compartment) and cultured overnight. H358 cells were seeded similarly at a density of 15×10⁴ cells/mL and cultured overnight. The cells were gently washed with DPBS, and treated for 2 h with fluorescein-labeled B4-27 (1 or 5 μM) in phenol-red free DMEM for HeLa and A549 cells or RPMI for H358 cells containing 1% FBS and 1% penicillin-streptomycin sulfate. After removal of the medium, the cells were gently washed with DPBS twice. Cells were imaged immediately on a Nikon A1R live-cell confocal laser scanning microscope (ECLIPSE Ti-E automated, inverted) equipped with a 20× or 100× oil objective (1.45 N.A.) and a heated (37° C.) chamber supplied with 5% CO₂. The data were analyzed using NIS-Elements AR.

For co-localization analysis, MDCK cells stably expressing GFP-KRasG12V were seeded in a 35-mm glass-bottomed microwell dish with 4 compartments (Greiner) at a density of 5×10⁴ cells/mL (300 μL in each compartment) and cultured overnight. The cells were gently washed with DPBS, and treated for 2 h with TMR-labeled B4-27 (3 or 5 μM) in phenol-red free DMEM containing 1% FBS and 1% penicillin-streptomycin sulfate. After incubation, the media was aspirated, and the cells were gently washed with DPBS twice before addition of fresh phenol-free media. Cells were imaged immediately on a Nikon A1R live-cell confocal laser scanning microscope (ECLIPSE Ti-E automated, inverted) equipped with a 100× oil objective (1.45 N.A.) and a heated (37° C.) chamber supplied with 5% CO₂. For the red channel (TMR), the laser line with 2Ex 561 nm was set at 0.5% laser power. For the green channel (GFP), the laser line with 2Ex 487 nm was set at 0.4% laser power. Images were acquired in Channel Series mode (i.e., each fluorescent channel was imaged independently, rather than simultaneously) to eliminate spectral bleed through. Images were denoised and analyzed using NIS-Elements AR. Colocalization on the membrane was analyzed inside the regions of interest (ROIs) using Pearson's correlation constant.

Flow Cytometry. HeLa, A549 and H358 cells were seeded in a 12-well plate at a density 1.5×10⁵ cells per well and cultured overnight. Next day, fluorescein-labeled B4-27 was added in DMEM or RPMI supplemented with 1% FBS and 1% penicillin-streptomycin sulfate and incubated at 37° C. for 2 h. The cells were washed with cold DPBS twice, detached from the plate with 0.25% trypsin, diluted into cold DPBS and pelleted at 300 g for 5 min at 4° C. The supernatant was discarded and the cells were washed twice with cold DPBS and re-suspended in 200 μL of cold DPBS. The samples were analyzed on a BD FACS LSR II flow cytometer. For the fluorescein-labelled peptides, a 488-nm laser was used for excitation, and the fluorescence was analyzed in the FITC channel.

Mouse Xenograft Models. All animal experiments were performed in compliance with institutional animal care guidelines and according to committee-approved protocols. For A549 xenografts, ˜1.5×10⁶ A549 cells were injected subcutaneously into 8-week-old nude mice. Tumors were allowed to grow to a size of ˜40 mm³, and six mice per group were treated with 100 μL injections of vehicle (1.5% (vol/vol) DMSO in saline) or B4-27 (1 or 5 mg/kg in 1.5% (vol/vol) DMSO in saline) via tail vein injection daily for 9 days and tumor volumes were measured every other day. After 9 days, mice were sacrificed and tumors were collected. Pictures of tumors were taken, and tumor weight was measured.

For H358 xenografts, ˜2×10⁶ H358 cells were injected subcutaneously into 8-week-old nude mice. Tumors were allowed to grow to a size of ˜35 mm³, and seven mice per group were treated with 100-μL injections of vehicle (1.5% (vol/vol) DMSO in saline) or B4-27 (5 mg/kg in 1.5% (vol/vol) DMSO in saline) via tail vein injection daily for 9 days. Body weights were recorded and monitored for any signs of toxicity. Tumors were measured every other day and excised and retained for further analysis on day 9.

Histology and Immunohistochemistry. Tumors were harvested from mice and fixed with 4% formaldehyde buffer for 24 h at RT. Paraffinized specimens were then sectioned (4-μm thick slices), and deparaffinized by xylenes (3×10 min) each followed by dipping in graded alcohols (100%, 95%, 80% and 70%) 5 min each. The slices were quenched for endogenous peroxidase activity with 3% H₂O₂ solution for 15 min and autoclaved at 100° C. for 30 min for antigen retrieval in citric acid buffer, pH 6.0. Sections were blocked with 10% normal goat serum for 30 min at RT and incubated with specific rabbit primary antibodies against Ki-67 (1:200 dilution, Abcam, ab16667) and p-ERK1/2 (1:200 dilution, Cell Signaling Technology, 4376) overnight at 4° C. Subsequently, the sections were incubated with Horse anti-Rabbit IgG secondary antibody (1:500 dilution, BA-1100, Vector labs, Burlingame, CA) for 30 min at RT. After incubation with avidin-biotin ABC complex (PK-4000, Vector labs, Burlingame, CA) followed by staining with DAB solution (SK-4105, Vector labs, Burlingame, CA), slides were washed thoroughly with tap water, counterstained with hematoxylin (H-3401, Vector labs, Burlingame, CA) for 5 s and dipped briefly in graded alcohols (70%, 80%, 95% and 100%), and in xylenes (2× 5 min). Finally, slides were mounted and imaged.

Using the above techniques, the following compounds were prepared and evaluated:

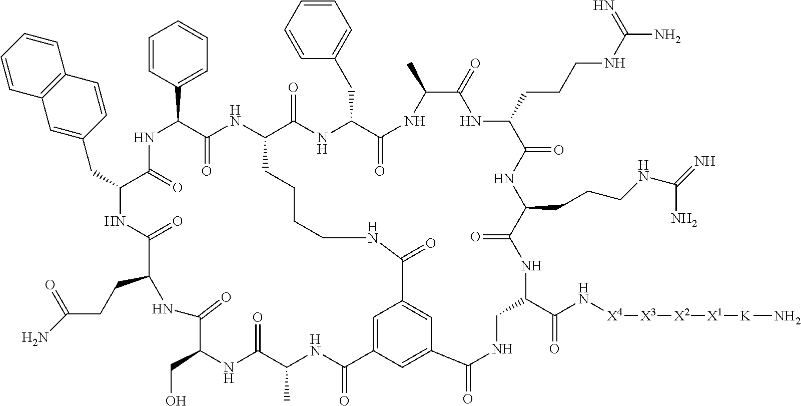

| | Hit Sequence | | | | HTRF IC₅₀ | MTT EC₅₀ |
|---|---|---|---|---|---|---|
| PEPTIDE | X⁴ | X³ | X² | X¹ | (μM)^{a} | (μM)^{a} |
| B3-4 | β-Ala | β-Ala | Lys | null | 4.4 ± 1.3 | 29 ± 6 |
| B3-4-1 | D-Tyr | Asp | Ser | D-Val | NA | NT |
| B3-4-2 | D-Glu | His | D-Pro | D-Tyr | 5.8 ± 1.1 | NA |
| B3-4-3 | D-Thr | Ser | D-Pro | Pip | 8.0 ± 1.0 | NT |
| B3-4-4 | Orn | D-Arg | Fpa | D-Ala | 1.7 ± 0.2 | 16.6 ± 4.5 |
| B3-4-5 | D-Thr | His | D-Glu | D-Arg | 6.9 ± 2.1 | NT |
| B3-4-6 | Ser | Gln | Ile | D-Arg | 9.7 ± 0.8 | NT |
| B3-4-7 | Gly | Gln | D-Leu | D-Arg | 1.7 ± 0.6 | NA |
| B3-4-8 | D-Leu | Ser | D-Nal | D-Arg | 17.5 ± 4.4 | NT |
| B3-4-9 | D-Asn | D-Val | D-Thr | D-Trp | NA | NT |
| B3-4-10 | D-Pro | D-Arg | D-Phe | D-Tyr | NA | NT |
| B3-4-11 | Nle | Gly | Nle | D-Arg | 7.2 ± 2.0 | 24.2 ± 6.6 |
| B3-4-12 | Ala | D-Arg | D-Phe | Fpa | 3.1 ± 0.6 | 18.9 ± 5.8 |
| B3-4-13 | Orn | D-Arg | D-β-homoAla | Fpa | 1.7 ± 0.1 | 30.0 ± 11.3 |
| B3-4-14 | D-Lys | His | D-Tyr | D-Glu | NA | NT |
| B3-4-15 | Orn | D-Val | D-β-homoAla | D-Trp | 2.9 ± 0.8 | NA |
| B3-4-16 | D-Pro | D-Arg | D-Nal | D-Glu | NA | NT |
| B3-4-17 | Orn | D-Asn | D-Tyr | D-Thr | 5.7 ± 0.1 | NA |

^{A}NT, not tested; NA, no significant activity; Fpa = L-4-fluorophenylalanine; Orn = L-ornithine; D-Nal = D-2-napthylalanine

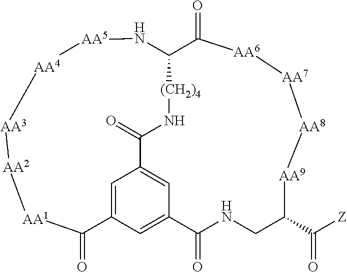

| Peptide | AA¹ | AA² | AA³ | AA⁴ | AA⁵ | AA⁶ | AA⁷ | AA⁸ | AA⁹ | Z |
|---|---|---|---|---|---|---|---|---|---|---|
| B3-4 | Φ | S | Q | Φ | Π | f | A | r | R | -BBK-NH₂ |
| B4 | Φ | S | Q | Φ | Π | f | A | r | R | -ΣrΨa-K-NH₂ |
| B4-1 | Φ | S | N | Φ | Π | f | A | r | R | -ΣrΨa-K-NH₂ |
| B4-2 | Φ | S | N | Φ | Π | f | A | r | R | -ΣrΨa-NH₂ |
| B4-3 | Φ | S | N | Φ | Π | f | A | r | R | -ΣrΨ-NH₂ |
| B4-4 | Φ | S | N | Φ | Π | f | A | r | R | Σr-NH₂ |
| B4-5 | Φ | S | N | Φ | Π | f | A | r | R | -Σ-NH₂ |
| B4-6 | Φ | S | N | Φ | Π | f | A | r | R | -ΣrFa-NH₂ |
| B4-7 | Φ | S | N | Φ | Π | f | A | r | R | -ΣrYa-NH₂ |
| B4-8 | Φ | S | N | Φ | Π | f | A | r | R | -ΣrRa-NH₂ |
| B4-9 | Φ | S | N | Φ | Π | f | A | r | R | -Σrra-NH₂ |
| B4-10 | Φ | S | N | Φ | Π | f | A | r | R | -ΣrZa-NH₂ |
| B4-11 | Φ | S | N | Φ | Π | f | A | r | R | -ΣrWa-NH₂ |
| B4-12 | Φ | S | N | Φ | Π | f | A | r | R | -Σrϑa-NH₂ |
| B4-13 | Φ | S | N | Φ | Π | f | A | r | R | -ΣrΠa-NH₂ |
| B4-14 | Φ | S | N | Φ | Π | f | A | r | R | -ΣrΓa-NH₂ |
| B4-15 | Φ | S | N | Φ | Π | f | A | r | R | -ΣrΛa-NH₂ |
| B4-16 | Φ | S | N | Φ | Π | f | A | r | R | -ΣrPa-NH₂ |
| B4-17 | Φ | S | N | Φ | Π | f | A | r | R | -KrΨa-NH₂ |
| B4-18 | Φ | S | N | Φ | Π | f | A | r | R | -ΘrΨa-NH₂ |
| B4-19 | Φ | S | N | Φ | Π | f | A | r | R | -ΞrΨa-NH₂ |
| B4-20 | Φ | S | N | Φ | Π | f | A | r | R | -NrΨa-NH₂ |
| B4-21 | Φ | S | N | Φ | Π | f | A | r | R | -QrΨa-NH₂ |
| B4-22 | Φ | S | N | Φ | Π | F | A | r | R | -ΣrFa-NH₂ |
| B4-23 | Φ | S | N | Φ | Π | f | a | r | R | -ΣrFa-NH₂ |
| B4-24 | Φ | S | N | Φ | Π | f | ζ | r | R | -ΣrFa-NH₂ |
| B4-25 | Φ | S | N | Φ | Π | f | L | r | R | -ΣrFa-NH₂ |
| B4-26 | Φ | S | N | Φ | Π | f | S | r | R | -ΣrFa-NH₂ |
| B4-27 | Φ | S | N | Φ | Π | f | Q | r | R | -ΣrFa-NH₂ |
| B4-28 | Φ | S | N | Φ | Π | f | H | r | R | -ΣrFa-NH₂ |
| B4-29 | Φ | S | N | Φ | Π | f | N | r | R | -ΣrFa-NH₂ |
| B4-30 | Φ | S | N | Φ | Π | f | Σ | r | R | -ΣrFa-NH₂ |
| B4-31 | Ω | S | N | Φ | Π | f | Q | r | R | -ΣrFa-NH₂ |
| B4-32 | Φ | S | N | Ω | Π | f | Q | r | R | -ΣrFa-NH₂ |
| B4-33 | Φ | N | N | Φ | Π | f | Q | r | R | -ΣrFa-NH₂ |
| B4-34 | Φ | T | N | Φ | Π | f | Q | r | R | -ΣrFa-NH₂ |
| B4-35 | Φ | ζ | N | Φ | Π | f | Q | r | R | -ΣrFa-NH₂ |
| B4-36 | Φ | S | A | Φ | Π | f | Q | r | R | -ΣrFa-NH₂ |
| B4-37 | Φ | S | S | Φ | Π | f | Q | r | R | -ΣrFa-NH₂ |
| B4-38 | Φ | S | N | Φ | Π | ψ | Q | r | R | -ΣrFa-NH₂ |
| B4-39 | Φ | S | N | Φ | Π | λ | Q | r | R | -ΣrFa-NH₂ |
| B4-40 | Φ | S | N | Φ | Π | f | Q | r | R | ΣrFa-NH₂ |
| B4-41 | Φ | S | N | Φ | Π | f | Q | r | R | -ΣrXa-NH₂ |
| B4-42 | Φ | S | N | Φ | Π | f | Q | r | R | -ΣrFv-NH₂ |
| B4-43 | Φ | S | N | Φ | Π | f | Q | r | R | -ΣrFt-NH₂ |
| B4-44 | Φ | S | N | Φ | Π | f | Q | r | R | -ΣrFs-NH₂ |
| B4-45 | Φ | S | N | Φ | Π | f | Q | r | R | -ΣrFn-NH₂ |
| B4-46 | Φ | S | N | Φ | Π | f | Q | r | R | -ΣrFB-NH₂ |

L-AA's are shown as the capitalized one letter code while D-AA's are lowercase. Π=L-phenylglycine; Φ=D-2-napthylalanine; Σ=L-ornithine; Γ=L-3,4-difluorophenylalanine; μ=3-cyclohexyl-L-alanine: Λ=L-3-chlorophenylalanine; λ=D-3-chlorophenylalanine: P=3-(2-pyridyl)-L-alanine: Ω=3-(3-benzo (b) thienyl)-D-alanine; Ψ=L-4-fluorophenylalanine: ψ=D-4-fluorophenylalanine: Z=L-homophenylalanine; Θ=L-2,3-diaminopropionic acid: Ξ=L-2,4-diaminobutyric acid; ζ=L-α-aminobutyric acid; ç=L-homoserine; B=β-alanine: X=L-β-homophenylalanine.

### Sequences and Ras-Binding Affinities of Cyclorasin B3 Analogs

| Peptide | K_{D} (μM) |
|---|---|
| B3 | 2.1 ± 0.9 |
| B3-1 | 0.58 ± 0.12 |
| B3-2 | 0.71 ± 0.14 |
| B3-3 | 0.61 ± 0.13 |
| B3-4 | 0.14 ± 0.06 |
| B3-5 | 1.8 ± 0.4 |
| B3-6 | 2.3 ± 0.3 |
| B3-7 | NA |
| B3-8 | 0.92 ± 0.13 |
| B3-9 | 0.45 ± 0.11 |
| B3-10 | NA |

NA = no significant activity

| | HTRF IC₅₀ | CELLTITER-GLO EC₅₀ |
|---|---|---|
| PEPTIDE | (μM) | (μM) |
| B3-4 | 4.36 ± 1.31 | 22.4 ± 3.7 |
| B4 | 1.72 ± 0.25 | 14.5 ± 2.7 |
| B4-1 | 0.89 ± 0.26 | 7.2 ± 0.8 |
| B4-2 | 0.70 ± 0.24 | 6.0 ± 1.6 |
| B4-3 | 0.55 ± 0.07 | 15.0 ± 1.9 |
| B4-4 | 0.70 ± 0.10 | 15.5 ± 4.4 |
| B4-5 | 1.29 ± 0.47 | 16.6 ± 1.5 |
| B4-6 | 0.51 ± 0.01 | 3.2 ± 1.5 |
| B4-7 | 1.06 ± 0.11 | 19.2 ± 10.1 |
| B4-8 | 1.01 ± 0.06 | 20.4 ± 1.0 |
| B4-9 | 0.70 ± 0.0 | 10.3 ± 8.0 |
| B4-10 | 0.77 ± 0.03 | 9.2 ± 1.1 |
| B4-11 | 2.23 ± 0.75 | NA |
| B4-12 | 0.94 ± 0.33 | 10.9 ± 4.0 |
| B4-13 | 0.76 ± 0.24 | 4.5 ± 3.0 |
| B4-14 | 0.50 ± 0.04 | 4.4 ± 0.3 |
| B4-15 | 0.33 ± 0.01 | 9.0 ± 0.7 |
| B4-16 | 0.41 ± 0.06 | 6.6 ± 0.4 |
| B4-17 | 1.05 ± 0.42 | 4.4 ± 1.5 |
| B4-18 | 1.67 ± 0.17 | NA |
| B4-19 | 0.87 ± 0.05 | 12.0 ± 7.8 |
| B4-20 | 0.28 ± 0.06 | 8.4 ± 5.2 |
| B4-21 | 0.23 ± 0.02 | 7.5 ± 4.0 |
| B4-22 | 0.65 ± 0.37 | 21.1 ± 1.1 |
| B4-23 | 0.52 ± 0.01 | 16.1 ± 8.4 |
| B4-24 | 0.61 ± 0.09 | 7.2 ± 2.7 |
| B4-25 | 0.79 ± 0.02 | 4.8 ± 1.1 |
| B4-26 | 0.56 ± 0.17 | 11.3 ± 8.0 |
| B4-27 | 0.69 ± 0.11 | 2.1 ± 0.1 |
| B4-28 | 1.04 ± 0.16 | 8.2 ± 4.8 |
| B4-29 | 1.25 ± 0.44 | 13.8 ± 3.1 |
| B4-30 | 0.83 ± 0.20 | 6.4 ± 0.8 |
| B4-31 | 0.64 ± 0.09 | 5.2 ± 1.1 |
| B4-32 | 0.71 ± 0.08 | 4.9 ± 1.2 |
| B4-33 | 0.86 ± 0.12 | 17.1 ± 4.9 |
| B4-34 | 1.31 ± 0.29 | 22.9 ± 16.8 |
| B4-35 | 1.16 ± 0.17 | 5.9 ± 0.5 |
| B4-36 | 1.04 ± 0.17 | NA |
| B4-37 | 0.75 ± 0.18 | 17.2 ± 5.6 |
| B4-38 | 0.55 ± 0.03 | 15.1 ± 5.2 |
| B4-39 | 0.71 ± 0.01 | 23.2 ± 6.9 |
| B4-40 | 0.56 ± 0.24 | 5.0 ± 0.4 |
| B4-41 | 0.71 ± 0.09 | 7.4 ± 0.3 |
| B4-42 | 0.61 ± 0.17 | 7.1 ± 1.9 |
| B4-43 | 0.57 ± 0.10 | 2.6 ± 1.2 |
| B4-44 | 0.75 ± 0.03 | 5.3 ± 1.4 |
| B4-45 | 2.23 ± 1.72 | 3.1 ± 0.8 |
| B4-46 | 1.09 ± 0.45 | 11.8 ± 3.4 |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

The disclosure provides for the following example Statements, the numbering of which is not to be construed as designating levels of importance:
1. A compound of Formula 1:

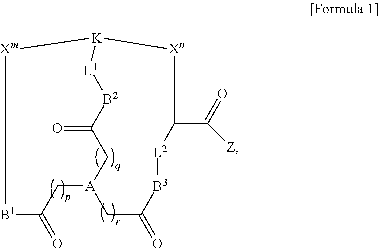

or a pharmaceutically acceptable salt thereof, wherein:
A is selected from N, aryl, C₃₋₈cycloalkyl, C₁₋₈ heterocyclyl, and C₁₋₈ heteroaryl;
p, q, and r are each independently selected from 0, 1, and 2;
B¹, B², and B³ are independently selected from null, O and NR¹;
   wherein R¹ comprises H, or substituted or unsubstituted C₁-C₈ alkyl;
L¹ is selected from substituted or unsubstituted C₁-C₆ alkyl;
L² is selected from substituted or unsubstituted C₁-C₆ alkyl;
X^{m} and Xⁿ independently comprise a sequence of 1-10 amino acids;
K has a structure represented by formula:

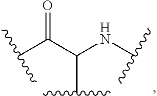 [SMILES: *NC(*)C(*)=O]

wherein each wavy indicates a point of attachment to one of X^{m}, Xⁿ, and L¹; and
Z comprises a sequence of 3-8 amino acids, OR^{π}, or NHR^{π},
wherein RT is selected from H, —(CH₂CH₂O)ₛH, and s is 1-100.
2. The compound of Statement 1, wherein Z has the formula:

*NH—X⁷—X⁶—X⁵—X⁴—X³—X²—X¹—X⁰—C(O)—X^{z}

wherein the asterisk represents the point of attachment to the compound of Formula 1,
X² is OR^{π}, or NHR^{π},
wherein RT is selected from H, —(CH₂CH₂O)ₛH, and s is 1-100.
X⁷ is null, a natural, or non-natural amino acid;
X⁶ is null, a natural, or non-natural amino acid;
X⁵ is null, a natural, or non-natural amino acid;
X⁴ is null, a natural, or non-natural amino acid;
X³ is a natural or non-natural amino acid;
X² is a natural or non-natural amino acid;
X¹ is a natural or non-natural amino acid; and
X⁰ is null, a natural, or non-natural amino acid.
3. The compound of Statement 1 or 2, wherein X⁰ is null, glycine, D-alanine, L-alanine, D-lysine, or L-lysine.
4. The compound of any preceding Statement, wherein X¹ is glycine, D-alanine, L-alanine, D-threonine, L-threonine, D-homoalanine, L-homoalanine, D-valine, L-valine, D-leucine, L-leucine, D-isoleucine, L-isoleucine, β-alanine, D-tert-butyl-alanine, L-tert-butyl-alanine, D-tert-butyl-glycine, L-tert-butyl-glycine, D-methyleucine, and L-methylleucine.
5. The compound of any preceding Statement, wherein X² is glycine, D-alanine, L-alanine, L-phenylalanine, D-phenylalanine, L-homophenyl alanine, D-homophenyl alanine, L-3-chlorophenylalanine, D-3-chlorophenylalanine, L-phenylglycine, D-phenylglycine, L-3,4-difluorophenylalanine, L-3-cyclohexylalanine, L-3-(2-pyridyl)-alanine, D-3,4-difluorophenylalanine, D-3-cyclohexylalanine, D-3-(2-pyridyl)-alanine, L-arginine, or D-arginine
6. The compound of any preceding Statement, wherein X³ is L-arginine, D-arginine, L-homoarginine, or D-homoarginine.
7. The compound of any preceding Statement, wherein X⁴ is D-ornithine, L-ornithine, D-lysine, L-lysine, D-arginine, L-arginine, D-asparagine, L-asparagine, D-glutamine, L-glutamine.
8. The compound of any preceding Statement, wherein X⁵, X⁶, and X⁷ are each null.
9. The compound of any preceding Statement, wherein Z has the formula:

*NH—X⁴—X³—X²—X¹—X⁰—C(O)—X^{z},
wherein the asterisk represents the point of attachment to the compound of Formula 1;
X² is OH or NH₂;
X⁴ is L-ornithine, D-ornithine, L-lysine, or D-lysine;
X³ is D-arginine;
X² is L-phenylalanine, D-phenylalanine, L-phenylglycine, or D-phenylglycine; and
X¹ is L-alanine, D-alanine, L-threonine, D-threonine, or glycine.
10. The compound of any preceding Statement, wherein Z has the formula:

*NH—X⁴—X³—X²—X¹—C(O)—X^{z},
wherein the asterisk represents the point of attachment to the compound of Formula 1;
X² is OH or NH₂;
X⁴ is L-ornithine;
X³ is D-arginine;
X² is L-phenylalanine; and
X¹ is D-alanine.
11. The compound according to any preceding Statement, wherein Z has the formula:

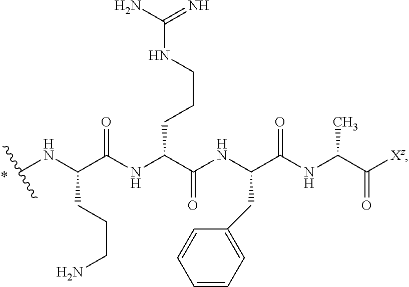

wherein the asterisk represents the point of attachment to the compound of Formula 1 and X² is
OH or NH₂.
12. The compound according to any preceding Statement, wherein A is phenyl, pyridinyl, or imidazolyl.
13. The compound according to any preceding Statement, wherein the compound of Formula 1 has the Formula 1-A:

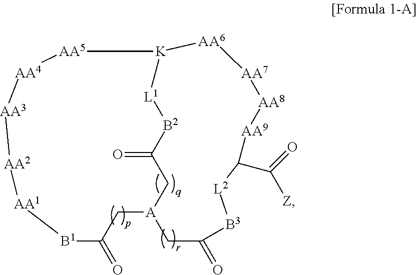

wherein AA¹, AA², AA³, AA⁴, AA⁵, AA⁶, AA⁷, AA⁸, and AA⁹ are each independently selected
from a natural or non-natural amino acid.
14. The compound of Formula 1-A of any preceding Statement, wherein each of AA¹, AA², AA⁴, and AA⁵ are L-amino acids.
15. The compound of Formula 1-A of any preceding Statement, wherein each of AA¹, AA², AA³, AA⁴, and AA⁵ are L-amino acids.
16. The compound of Formula 1-A of any preceding Statement, wherein AA⁶ and AA⁸ are L-amino acids.
17. The compound of Formula 1-A of any preceding Statement, wherein each of AA¹, AA², AA³, AA⁴, AA⁵, AA⁶, and AA⁸ are L-amino acids.
18. The compound of Formula 1-A of any preceding Statement, wherein AA⁷ and AA⁹ are D-amino acids.
19. The compound of Formula 1-A of any preceding Statement, wherein AA¹ is selected from D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, and L-cyclohexylalanine.
20. The compound of Formula 1-A of any preceding Statement, wherein AA² is selected from D-serine, L-serine, D-threonine, L-threonine, D-cysteine, L-cysteine, D-methionine, and L-methionine.
21. The compound of Formula 1-A of any preceding Statement, wherein AA³ is selected from D-glutamine, L-glutamine, D-asparagine, and L-asparagine.
22. The compound of Formula 1-A of any preceding Statement, wherein AA⁴ is selected from D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, and L-cyclohexylalanine.
23. The compound of Formula 1-A of any preceding Statement, wherein AA⁵ is selected from L-phenylglycine, D-phenylglycine, D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, L-cyclohexylalanine, D-tert-butylglycine, L-tert-butylglycine, D-valine, L-valine, D-leucine, L-leucine, D-alanine, L-alanine, D-isoleucine, L-isoleucine.
24. The compound of Formula 1-A of any preceding Statement, wherein AA⁶ is L-phenylglycine, D-phenylglycine, D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, L-cyclohexylalanine, D-tert-butylglycine, L-tert-butylglycine, D-valine, L-valine, D-leucine, L-leucine, D-alanine, L-alanine, D-isoleucine, L-isoleucine.
25. The compound of Formula 1-A of any preceding Statement, wherein AA⁷ is selected from D-glutamine, L-glutamine, D-homoglutamine, L-homoglutamine, D-asparagine, and L-asparagine.
26. The compound of any preceding Statement, wherein AA⁸ is selected from D-arginine, L-arginine, D-histidine, L-histidine, D-lysine, L-lysine, D-ornithine, L-ornithine, D-2,3-diaminopropionic acid, L-2,3-diaminopropionic acid, D-homoarginine, L-homoarginine, D-homolysine, L-homolysine, D-2,4-diaminobutyric acid, and L-2,4-diaminobutyric acid.
27. The compound of Formula 1-A of any preceding Statement, wherein AA⁹ is selected from D-arginine, L-arginine, D-histidine, L-histidine, D-lysine, L-lysine, D-ornithine, L-ornithine, D-2,3-diaminopropionic acid, L-2,3-diaminopropionic acid, D-homoarginine, L-homoarginine, D-homolysine, L-homolysine, D-2,4-diaminobutyric acid, and L-2,4-diaminobutyric acid.
28. The compound of Formula 1-A of any preceding Statement, wherein AA¹ is selected from L-naphthylalanine, L-phenylalanine, L-4-fluorophenylalanine, L-3,4-difluorophenylalanine, L-2,4-difluorophenylalanine, L-cyclohexylglycine, L-naphthylglycine, and L-cyclohexylalanine. In further embodiments, AA¹ is selected from L-naphthylalanine, and L-naphthylglycine.
29. The compound of Formula 1-A of any preceding Statement, wherein AA² is selected from L-serine, L-threonine, L-cysteine, and L-methionine. In certain embodiments, AA² is selected from L-serine and L-threonine,
30. The compound of Formula 1-A of any preceding Statement, wherein AA³ is selected from L-glutamine and L-asparagine.
31. The compound of Formula 1-A of any preceding Statement, wherein AA⁴ is selected from L-naphthylalanine, L-phenylalanine, L-4-fluorophenylalanine, L-3,4-difluorophenylalanine, L-2,4-difluorophenylalanine, L-cyclohexylglycine, L-naphthylglycine, and L-cyclohexylalanine. In further embodiments, AA⁴ is selected from L-naphthylalanine, and L-naphthylglycine.
32. The compound of Formula 1-A of any preceding Statement, wherein AA⁵ is selected from L-phenylglycine, L-phenylalanine, L-cyclohexylglycine, L-cyclohexylalanine.
33. The compound of Formula 1-A of any preceding Statement, wherein AA⁶ is D-phenylalanine.
34. The compound of Formula 1-A of any preceding Statement, wherein AA⁷ is selected from D-glutamine, D-homoglutamine, and D-asparagine.
35. The compound of Formula 1-A of any preceding Statement, wherein AA⁸ is selected from L-arginine, L-histidine, and L-homoarginine.
36. The compound of Formula 1-A of any preceding Statement, wherein AA⁹ is selected from D-arginine, D-histidine, D-lysine, D-ornithine, D-2,3-diaminopropionic acid, D-homoarginine, D-homolysine, and D-2,4-diaminobutyric acid.
37. The compound of Formula 1-A of any preceding Statement, L¹ is an unsubstituted n-butylene chain and L² is an unsubstituted methylene chain.
38. The compound of Formula 1-A of any preceding Statement, L¹ is an unsubstituted n-butylene chain and L² is an unsubstituted n-butylene chain.
39. The compound of Formula 1-A of any preceding Statement, L¹ is an unsubstituted methylene chain and L² is an unsubstituted n-butylene chain.
40. The compound of Formula 1-A of any preceding Statement, L¹ is an unsubstituted methylene chain and L² is an unsubstituted methylene chain.
41. The compound of Formula 1-A of any preceding Statement, having the formula:

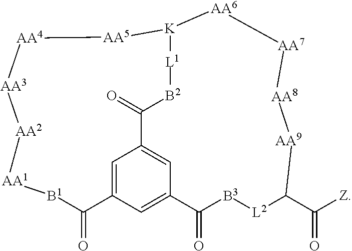

42. The compound of Formula 1-A of any preceding Statement, having the formula:

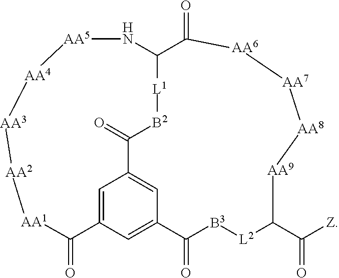

43. The compound of Formula 1-A of any preceding Statement, having the formula:

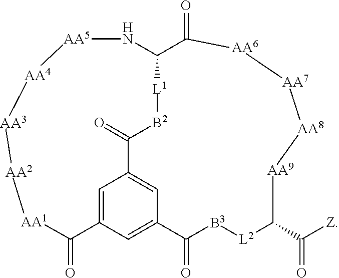

44. The compound of Formula 1-A of any preceding Statement, having the formula:

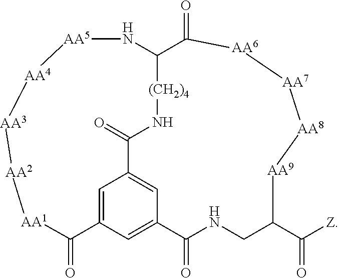

45. The compound of Formula 1-A of any preceding Statement, having the formula:

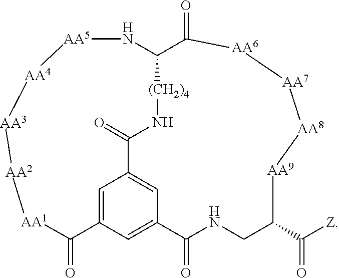

46. The compound according to any preceding Statement, wherein the compound of Formula 1 has the Formula 1-B:

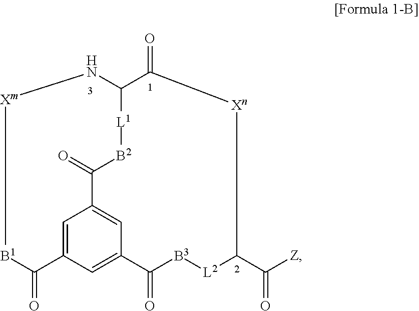

wherein Xⁿ has the formula:

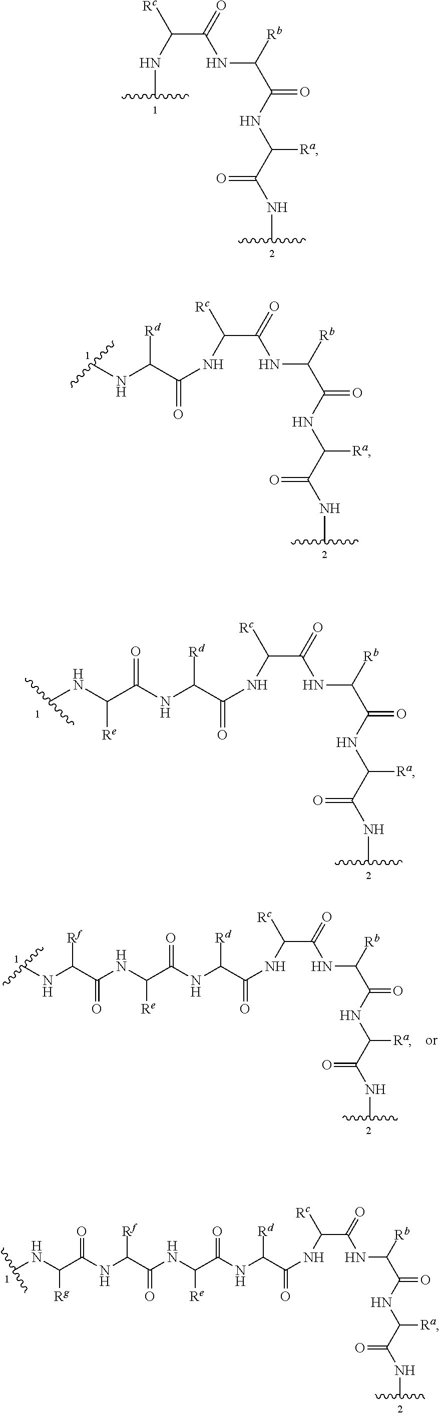

wherein wavy line 1 indicates the point of attachment to carbon I in Formula 1-B, wavy line 2 indicates the point of attachment to carbon 2 in Formula 1-B.

R^{a} has the formula:

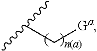

wherein n(a) is from 0-6, and G^{a} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, or CH(OH)CH₃.

R_{b} has the formula:

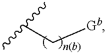

wherein n(b) is from 0-6, and G^{b} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{c} has the formula:

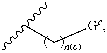

wherein n(c) is from 0-6, and G^{c} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{d} has the formula:

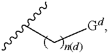

wherein n(d) is from 0-6, and G^{d} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{c} has the formula:

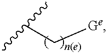

wherein n(e) is from 0-6, and G^{c} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{f} has the formula:

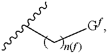

wherein n(b) is from 0-6, and G^{f} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{g} has the formula:

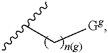

wherein n(g) is from 0-6, and G^{g} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃;
X^{m} has the formula:

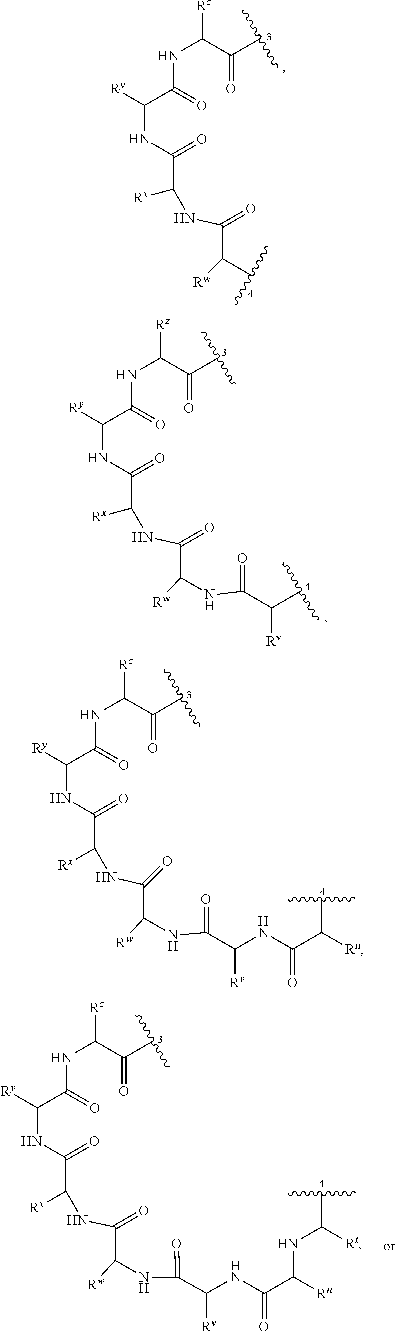

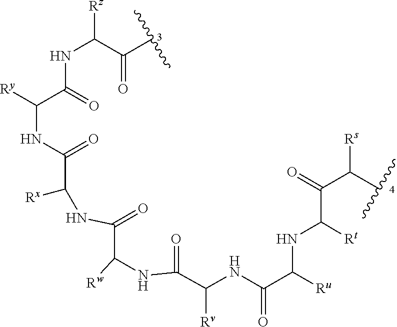

wherein wavy line 3 indicates the point of attachment to nitrogen 3 in Formula 1-b, wavy line 4 indicates the point of attachment to B1,
R^{z} has the formula:

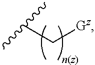

wherein n(z) is from 0-6, and G^{z} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{y} has the formula:

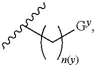

wherein n(y) is from 0-6, and G^{y} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{x} has the formula:

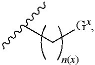

wherein n(x) is from 0-6, and G^{x} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{w} has the formula:

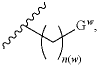

wherein n(w) is from 0-6, and G^{w} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{v} has the formula:

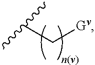

wherein n(v) is from 0-6, and G^{v} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{u} has the formula:

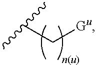

wherein n(u) is from 0-6, and G^{u} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R′ has the formula:

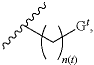

wherein n(t) is from 0-6, and G^{t} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

R^{s} has the formula:

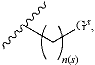

wherein n(s) is from 0-6, and G^{s} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
47. The compound of Formula 1-B of any preceding Statement, wherein G^{a} is methyl, isopropyl, n-butyl, n-propyl, 3-methylprop-1-yl, 2-methylprop-1-yl, 1-napthyl, phenyl, 2-naphthyl, 3-naphthyl, or cyclohexyl.
48. The compound of Formula 1-B of any preceding Statement, wherein G^{a} has the formula:

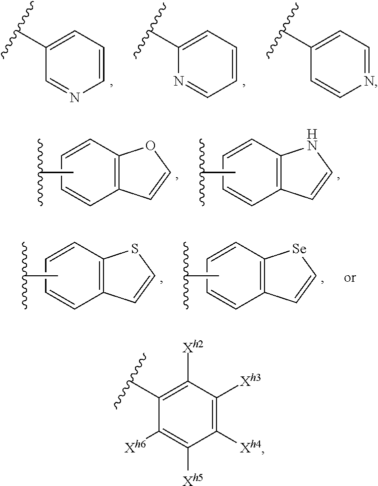

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
49. The compound of Formula 1-B of any preceding Statement, wherein n(a) is 1, 2, or 3, and G^{a} is CONH₂, NH₂, or NHC(═NH)NH₂.
50. The compound of Formula 1-B of any preceding Statement, wherein G^{a} is NHC(═NH)NH₂.
51. The compound of Formula 1-B of any preceding Statement, wherein G^{b} is methyl, isopropyl, n-butyl, n-propyl, 3-methylprop-1-yl, 2-methylprop-1-yl, 1-napthyl, 2-napthyl, 3-napthyl, or cyclohexyl.
52. The compound of Formula 1-B of any preceding Statement, wherein G^{b} has the formula:

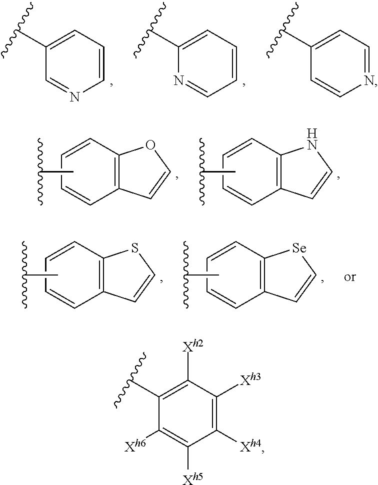

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
53. The compound of Formula 1-B of any preceding Statement, wherein n(b) is 1, 2, or 3, and G^{b} is CONH₂, NH₂, or NHC(═NH)NH₂.
54. The compound of Formula 1-B of any preceding Statement, wherein G^{b} is NHC(═NH)NH₂.
55. The compound of Formula 1-B of any preceding Statement, wherein G^{c} is methyl, isopropyl, n-butyl, n-propyl, 3-methylprop-1-yl, 2-methylprop-1-yl, 1-naphthyl, 2-naphthyl, 3-naphthyl, or cyclohexyl.
56. The compound of Formula 1-B of any preceding Statement, wherein G^{c} has the formula:

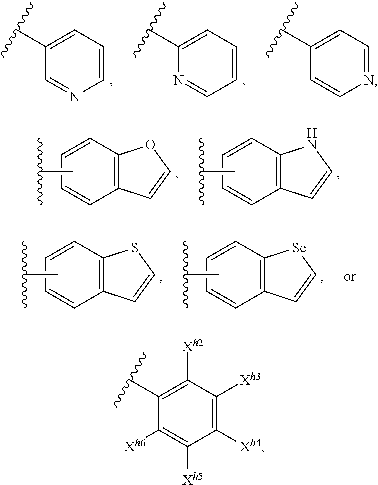

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
57. The compound of Formula 1-B of any preceding Statement, wherein n(c) is 1, 2, or 3, and G^{c} is CONH₂, NH₂, NHC(═NH)NH₂.
58. The compound of Formula 1-B of any preceding Statement, wherein G^{c} is CONH₂.
59. The compound of Formula 1-B of any preceding Statement, wherein G^{d} is methyl, isopropyl, n-butyl, n-propyl, 3-methylprop-1-yl, 2-methylprop-1-yl, 1-napthyl, 2-naphthyl, 3-naphthyl, or cyclohexyl.
60. The compound of Formula 1-B of any preceding Statement, wherein G^{d} has the formula:

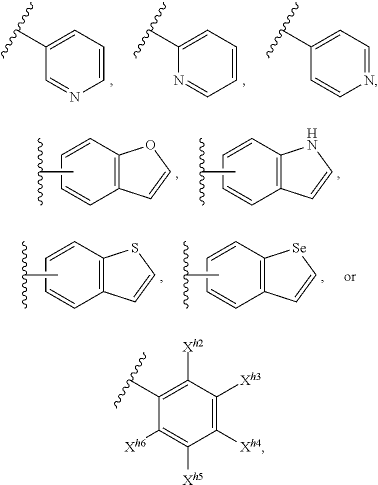

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
61. The compound of Formula 1-B of any preceding Statement, wherein n(d) is 0 or 1, and G^{d} is has the formula:

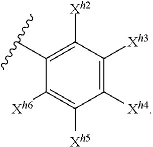

wherein: X^{h2}, X^{h3}, X^{h4}, X^{h5}, and X^{h6} are each hydrogen;
X^{h4} is F or Cl, and X^{h2}, X^{h3}, X^{h5}, and X^{h6} are each hydrogen; or X^{h3} is F or Cl, X^{h4} is F or Cl, and X^{h2}, X^{h5}, and X^{h6} are each hydrogen.
62. The compound of Formula 1-B of any preceding Statement, wherein G^{e} is methyl, isopropyl, n-butyl, n-propyl, 3-methylprop-1-yl, 2-methylprop-1-yl, 1-naphthyl, 2-naphthyl, 3-naphthyl, or cyclohexyl.
63. The compound of Formula 1-B of any preceding Statement, wherein G^{e} has the formula:

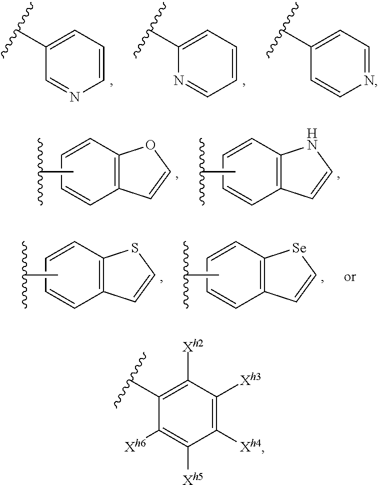

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
64. The compound of Formula 1-B of any preceding Statement, wherein G^{f} is methyl, isopropyl, n-butyl, n-propyl, 3-methylprop-1-yl, 2-methylprop-1-yl, 1-naphthyl, 2-naphthyl, 3-naphthyl, or cyclohexyl.
65. The compound of Formula 1-B of any preceding Statement, wherein G^{f} has the formula:

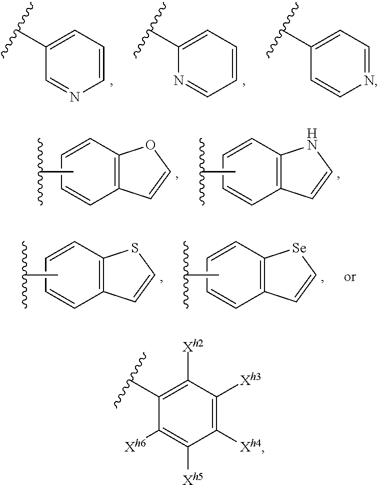

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
66. The compound of Formula 1-B of any preceding Statement, wherein G8 is methyl, isopropyl, n-butyl, n-propyl, 3-methylprop-1-yl, 2-methylprop-1-yl, 1-naphthyl, 2-naphthyl, 3-naphthyl, or cyclohexyl.
67. The compound of Formula 1-B of any preceding Statement, wherein G8 has the formula:

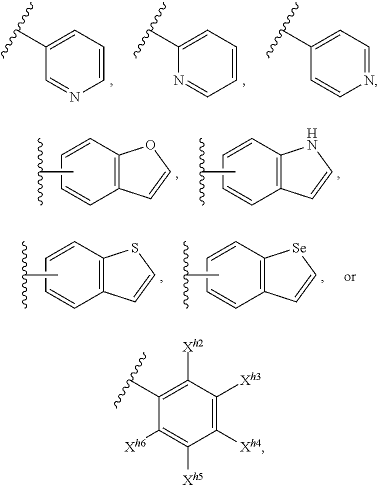

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
68. The compound of Formula 1-B of any preceding Statement, wherein G^{z} is methyl, isopropyl, n-butyl, n-propyl, 3-methylprop-1-yl, 2-methylprop-1-yl, 1-naphthyl, 2-naphthyl, 3-naphthyl, or cyclohexyl.
69. The compound of Formula 1-B of any preceding Statement, wherein G^{z} has the formula:

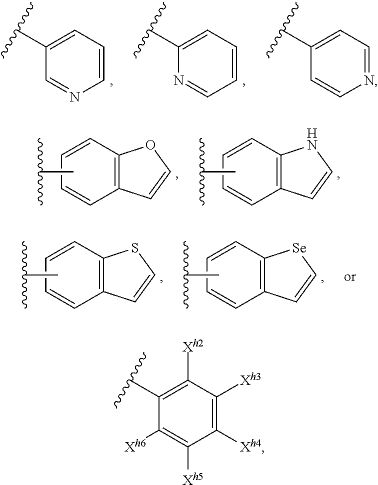

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
70. The compound of Formula 1-B of any preceding Statement, wherein n(z) is 0 or 1, and G^{z} is has the formula:

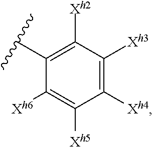

wherein: X^{h2}, X^{h3}, X^{h4}, X^{h5}, and X^{h6} are each hydrogen;
X^{h4} is F or Cl, and X^{h2}, X^{h3}, X^{h5}, and X^{h6} are each hydrogen; or
X^{h3} is F or Cl, X^{h4} is F or Cl, and X^{h2}, X^{h5}, and X^{h6} are each hydrogen.
71. The compound of Formula 1-B of any preceding Statement, wherein G^{y} is methyl, isopropyl, n-butyl, n-propyl, 3-methylprop-1-yl, 2-methylprop-1-yl, 1-naphthyl, 2-naphthyl, 3-naphthyl, or cyclohexyl.
72. The compound of Formula 1-B of any preceding Statement, wherein G^{y} has the formula:

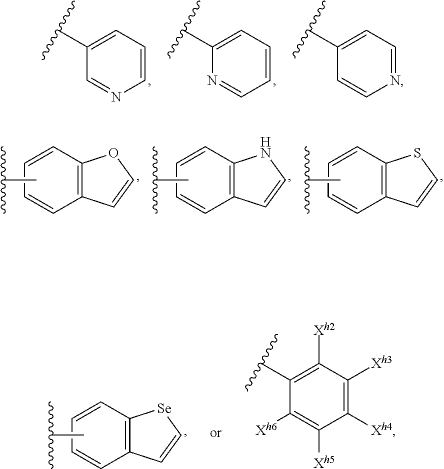

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
73. The compound of Formula 1-B of any preceding Statement, wherein G^{x} is methyl, isopropyl, n-butyl, n-propyl, 3-methylprop-1-yl, 2-methylprop-1-yl, 1-naphthyl, 2-naphthyl, 3-naphthyl, or cyclohexyl.
74. The compound of Formula 1-B of any preceding Statement, wherein n(x) is 1, 2, or 3, and G^{x} is CONH₂, NH₂, NHC(═NH)NH₂.
75. The compound of Formula 1-B of any preceding Statement, wherein G is CONH₂.
76. The compound of Formula 1-B of any preceding Statement, wherein G^{w} is methyl, isopropyl, n-butyl, n-propyl, 3-methylprop-1-yl, 2-methylprop-1-yl, 1-naphthyl, 2-naphthyl, 3-naphthyl, or cyclohexyl.
77. The compound of Formula 1-B of any preceding Statement, wherein G^{w} has the formula:

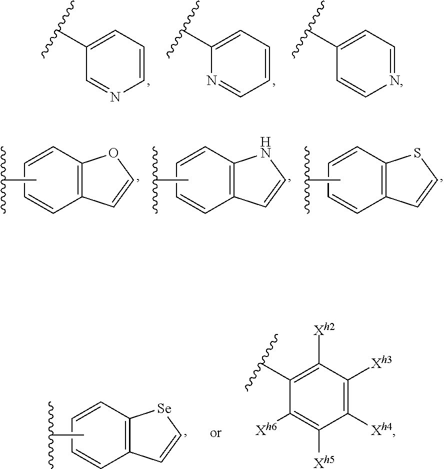

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
78. The compound of Formula 1-B of any preceding Statement, wherein n(w) is 0 and G^{w} is CH(CH₃)OH.
79. The compound of Formula 1-B of any preceding Statement, wherein N (w) is 1, and G^{w} is OH.
80. The compound of Formula 1-B of any preceding Statement, wherein G^{v} is methyl, isopropyl, n-butyl, n-propyl, 3-methylprop-1-yl, 2-methylprop-1-yl, 1-naphthyl, 2-naphthyl, 3-naphthyl, or cyclohexyl. 81. The compound of Formula 1-B of any preceding Statement, wherein G^{v} has the formula:

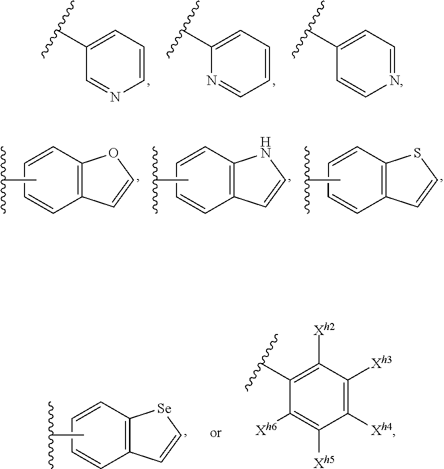

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

82. The compound of Formula 1-B of any preceding Statement, wherein n(v) is 0 or 1 and G^{v} has the formula:

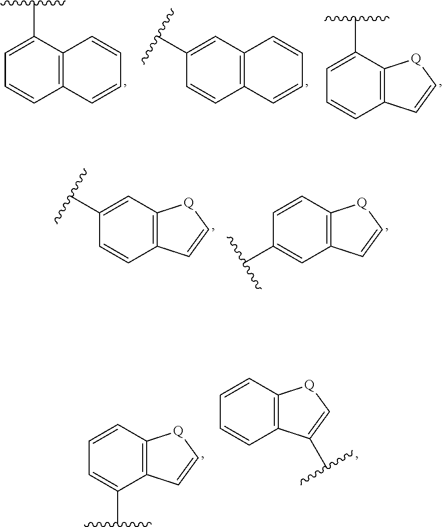

wherein Q is O, NH, S, or Se.
83. The compound of Formula 1-B of any preceding Statement, wherein G^{u} is methyl, isopropyl, n-butyl, n-propyl, 3-methylprop-1-yl, 2-methylprop-1-yl, 1-naphthyl, 2-naphthyl, 3-naphthyl, or cyclohexyl.
84. The compound of Formula 1-B of any preceding Statement, wherein G^{u} has the formula:

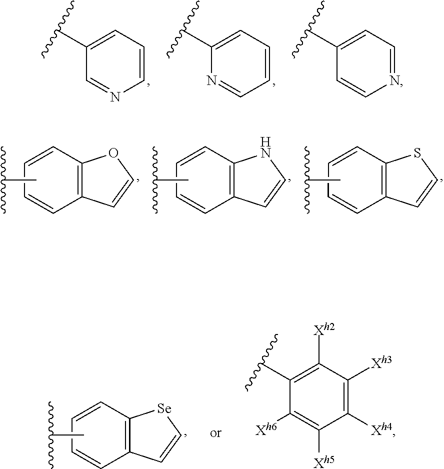

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
85. The compound of Formula 1-B of any preceding Statement, wherein G^{t} is methyl, isopropyl, n-butyl, n-propyl, 3-methylprop-1-yl, 2-methylprop-1-yl, 1-naphthyl, 2-naphthyl, 3-naphthyl, or cyclohexyl.
86. The compound of Formula 1-B of any preceding Statement, wherein G^{t} has the formula:

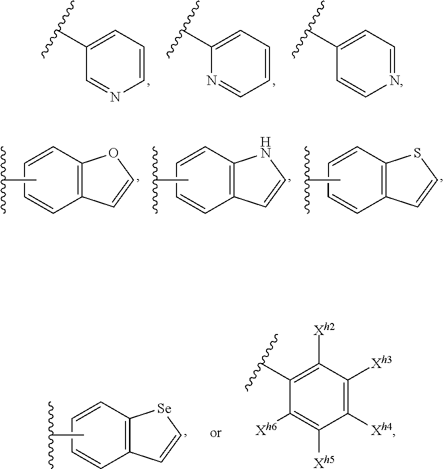

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
87. The compound of Formula 1-B of any preceding Statement, wherein G′ is methyl, isopropyl, n-butyl, n-propyl, 3-methylprop-1-yl, 2-methylprop-1-yl, 1-naphthyl, 2-naphthyl, 3-naphthyl, or cyclohexyl.
88. The compound of any preceding Statement, wherein G′ has the formula:

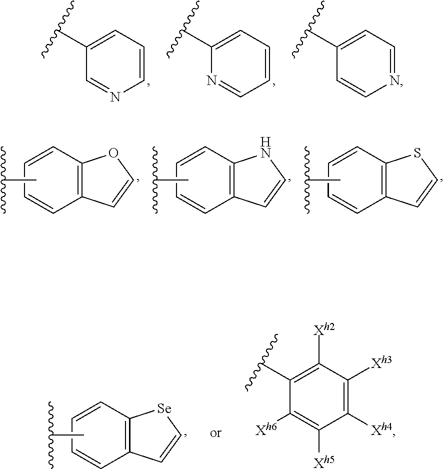

wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.
89. The compound of Formula 1-B of any preceding Statement, wherein X^{m} has the formula:

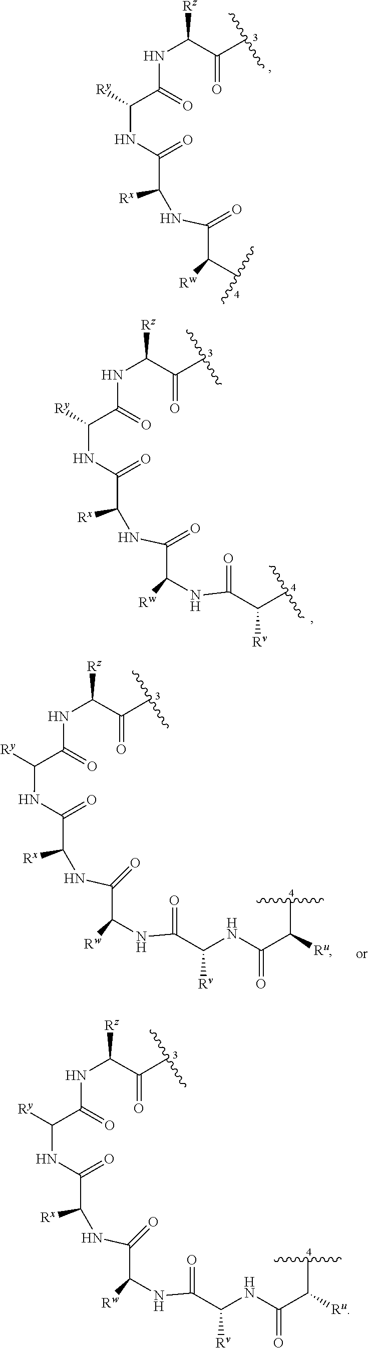

The compound of Formula 1-B of any preceding Statement, wherein X^{m} has the formula:

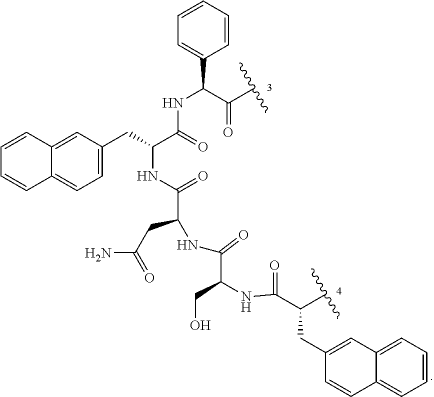 [SMILES: *C(=O)[C@@H](NC(=O)[C@@H](Cc1ccc2ccccc2c1)NC(=O)[C@H](CC(N)=O)NC(=O)[C@H](CO)NC(=O)[C@H](*)Cc1ccc2ccccc2c1)c1ccccc1]

90. The compound of Formula 1-B of any preceding Statement, wherein Xⁿ has the formula:

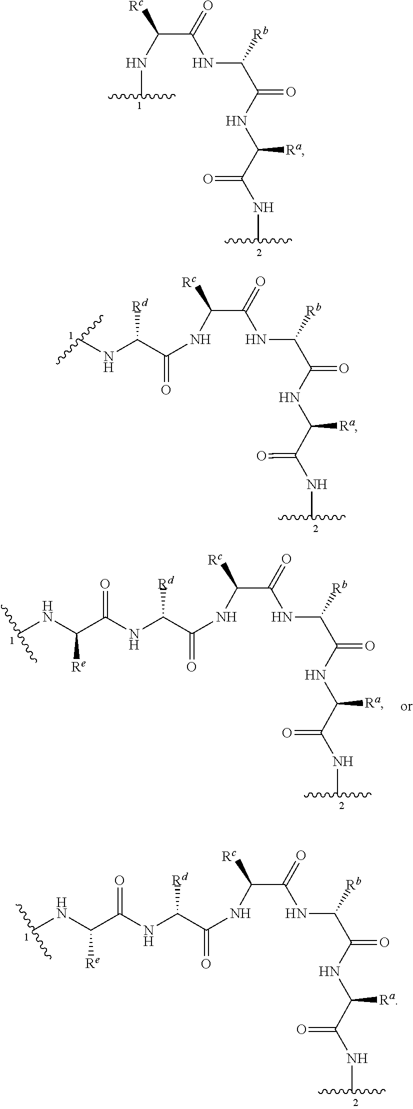

91. The compound of Formula 1-B of any preceding Statement, wherein Xⁿ has the formula:

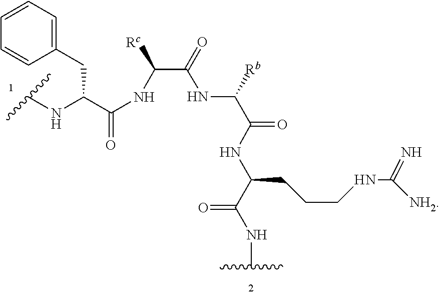

92. The compound of Formula 1-B of any preceding Statement, having the formula:

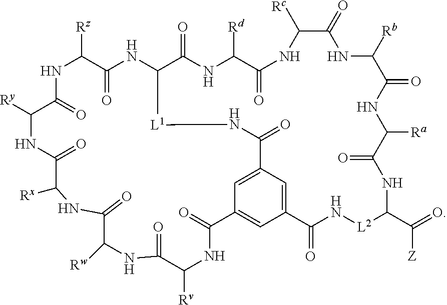

93. The compound of Formula 1-B of any preceding Statement, having the formula:

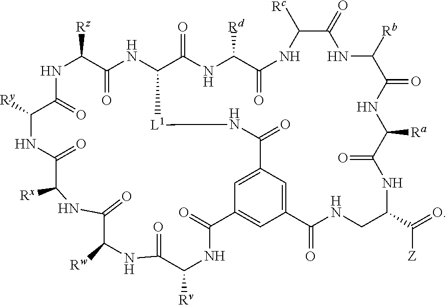

94. The compound of Formula 1-B of any preceding Statement, having the formula:

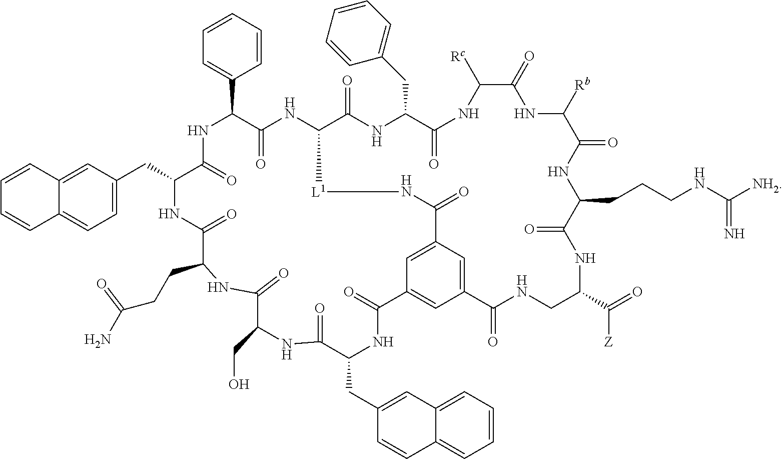

95. The compound of Formula 1-B of any preceding Statement, having the formula:

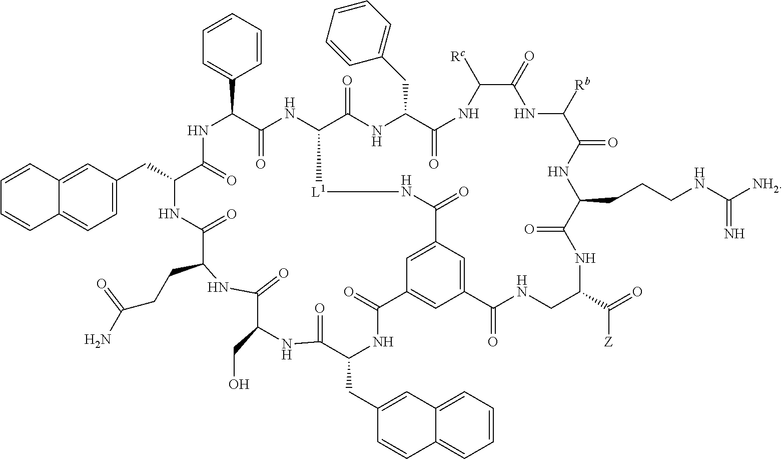

96. The compound of Formula 1-B of any preceding Statement, having the formula:

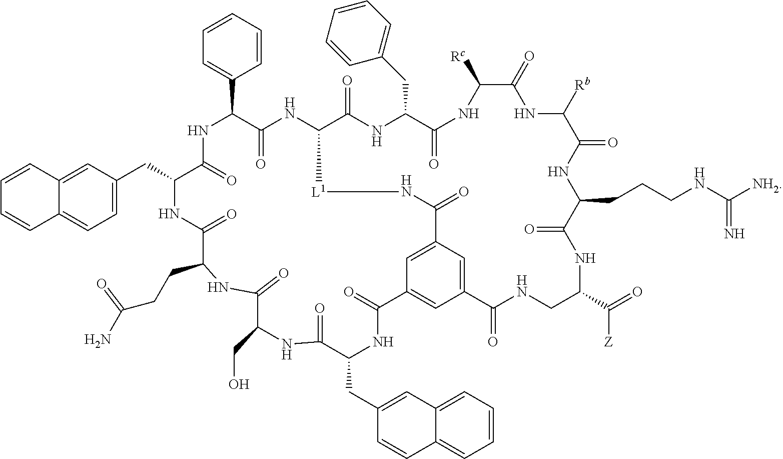

97. A pharmaceutical composition, comprising the compound of any preceding Statement and a pharmaceutically acceptable excipient.
98. A method of treating cancer, comprising administering to a patient in need thereof a compound or composition of any preceding Statement.
99. A method of treating cancer, comprising administering to a patient in need thereof a compound or composition of any preceding Statement, wherein the cancer is characterized by overexpression, activation, amplification, or mutation of Ras.
100. A method of treating cancer, comprising administering to a patient in need thereof a compound or composition of any preceding Statement, wherein the cancer is characterized by overexpression, activation, amplification, or mutation of KRas, HRas, or NRas.
101. A method of treating cancer, comprising administering to a patient in need thereof a compound or composition of any preceding Statement, wherein the cancer is characterized by overexpression, activation, amplification, or mutation of Ras at Gly12, Gly13, or Gln61.
102. A method of treating cancer in a patient in need thereof, comprising:
   a) determining if the cancer is associated with one or more overexpression, activation, amplification, or mutation of Ras; and
   b) administering to the patient in need thereof a compound or composition of any preceding Statement.

1-103. (canceled)

104. A compound of Formula 1: 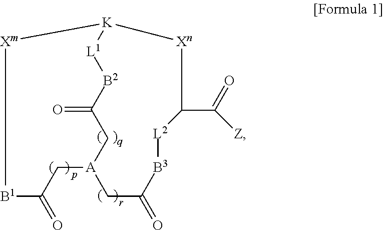
or a pharmaceutically acceptable salt thereof, wherein:
A is selected from N, aryl, C₃₋₈cycloalkyl, C₁₋₈ heterocyclyl, and C₁₋₈ heteroaryl;
p, q, and r are each independently selected from 0, 1, and 2;
B1, B2, and B3 are independently selected from null, O and NR¹;
wherein R¹ is in each case independently H, substituted C₁-C₈ alkyl, or unsubstituted C₁-C₈ alkyl;
L¹ is selected from substituted C₁-C₆ alkyl or unsubstituted C₁-C₆ alkyl;
L² is selected from substituted C₁-C₆ alkyl or unsubstituted C₁-C₆ alkyl;
X^{m} and Xⁿ independently comprise a sequence of 1-10 amino acids;
K has a structure represented by formula: 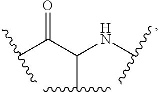 [SMILES: *NC(*)C(*)=O]
wherein each wavy indicates a point of attachment to one of X^{m}, Xⁿ, and L¹; and
Z comprises a sequence of 3-8 amino acids, OR^{π}, or NHR^{π},
wherein R^{π} is selected from H, —(CH₂CH₂O)ₛH, and s is 1-100.

105. The compound of claim 104, wherein Z has the formula:
(SEQ ID NO: 1)
*NH-X⁷-X⁶-X⁵-X⁴-X³-X²-X¹-X⁰-C(O)-X^{z},
wherein the asterisk represents the point of attachment to the compound of Formula 1,
X^{z} is OR^{π}, or NHR^{π},
wherein RT is selected from H, —(CH₂CH₂O) SH, and s is 1-100,
X⁷ is null, a natural, or non-natural amino acid;
X⁶ is null, a natural, or non-natural amino acid;
X⁵ is null, a natural, or non-natural amino acid;
X⁴ is null, a natural, or non-natural amino acid;
X³ is a natural or non-natural amino acid;
X² is a natural or non-natural amino acid;
X¹ is a natural or non-natural amino acid; and
X⁰ is null, a natural, or non-natural amino acid.

106. The compound of claim 105, wherein:
X⁰ is null, glycine, D-alanine, L-alanine, D-lysine, or L-lysine;
X¹ is glycine, D-alanine, L-alanine, D-threonine, L-threonine, D-homoalanine, L-homoalanine, D-valine, L-valine, D-leucine, L-leucine, D-isoleucine, L-isoleucine, β-alanine, D-tert-butyl-alanine, L-tert-butyl-alanine, D-tert-butyl-glycine, L-tert-butyl-glycine, D-methyleucine, and L-methylleucine;
X² is glycine, D-alanine, L-alanine, L-phenylalanine, D-phenylalanine, L-homophenyl alanine, D-homophenyl alanine, L-3-chlorophenylalanine, D-3-chlorophenylalanine, L-phenylglycine, D-phenylglycine, L-3,4-difluorophenylalanine, L-3-cyclohexylalanine, L-3-(2-pyridyl)-alanine, D-3,4-difluorophenylalanine, D-3-cyclohexylalanine, D-3-(2-pyridyl)-alanine, L-arginine, or D-arginine;
X³ is L-arginine, D-arginine, L-homoarginine, or D-homoarginine;
X⁴ is D-ornithine, L-ornithine, D-lysine, L-lysine, D-arginine, L-arginine, D-asparagine, L-asparagine, D-glutamine, L-glutamine; and
X⁵, X⁶, and X⁷ are each null.

107. The compound of claim 106, wherein Z has the formula:
(SEQ ID NO: 3)
*NH-X⁴-X³-X²-X¹-C(O)-X^{z},
wherein the asterisk represents the point of attachment to the compound of Formula 1;
X² is OH or NH₂;
X⁴ is L-ornithine;
X³ is D-arginine;
X² is L-phenylalanine; and
X¹ is D-alanine.

108. The compound of claim 104, wherein the compound of Formula 1 has the Formula 1-A: 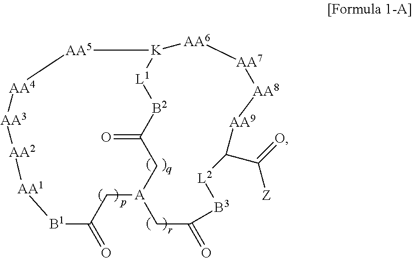
wherein AA¹, AA², AA³, AA⁴, AA⁵, AA⁶, AA⁷, AA⁸, and AA⁹ are each independently selected from a natural or non-natural amino acid.

109. The compound of claim 108, wherein
AA¹ is selected from D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, and L-cyclohexylalanine;
AA² is selected from D-serine, L-serine, D-threonine, L-threonine, D-cysteine, L-cysteine, D-methionine, and L-methionine;
AA³ is selected from D-glutamine, L-glutamine, D-asparagine, and L-asparagine;
AA⁴ is selected from D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, and L-cyclohexylalanine;
AA⁵ is selected from L-phenylglycine, D-phenylglycine, D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, L-cyclohexylalanine, D-tert-butylglycine, L-tert-butylglycine, D-valine, L-valine, D-leucine, L-leucine, D-alanine, L-alanine, D-isoleucine, L-isoleucine;
AA⁶ is L-phenylglycine, D-phenylglycine, D-naphthylalanine, L-naphthylalanine, D-phenylalanine, L-phenylalanine, D-tyrosine, L-tyrosine, D-tryptophan, L-tryptophan, D-4-fluorophenylalanine, L-4-fluorophenylalanine, D-3,4-difluorophenylalanine, L-3,4-difluorophenylalanine, D-2,4-difluorophenylalanine, L-2,4-difluorophenylalanine, D-cyclohexylglycine, L-cyclohexylglycine, D-naphthylglycine, L-naphthylglycine, D-cyclohexylalanine, L-cyclohexylalanine, D-tert-butylglycine, L-tert-butylglycine, D-valine, L-valine, D-leucine, L-leucine, D-alanine, L-alanine, D-isoleucine, L-isoleucine;
AA⁷ is selected from D-glutamine, L-glutamine, D-homoglutamine, L-homoglutamine, D-asparagine, and L-asparagine;
AA⁸ is selected from D-arginine, L-arginine, D-histidine, L-histidine, D-lysine, L-lysine, D-ornithine, L-ornithine, D-2,3-diaminopropionic acid, L-2,3-diaminopropionic acid, D-homoarginine, L-homoarginine, D-homolysine, L-homolysine, D-2,4-diaminobutyric acid, and L-2,4-diaminobutyric acid; and
AA⁹ is selected from D-arginine, L-arginine, D-histidine, L-histidine, D-lysine, L-lysine, D-ornithine, L-ornithine, D-2,3-diaminopropionic acid, L-2,3-diaminopropionic acid, D-homoarginine, L-homoarginine, D-homolysine, L-homolysine, D-2,4-diaminobutyric acid, and L-2,4-diaminobutyric acid.

110. The compound of claim 104, wherein:
L¹ is an unsubstituted n-butylene chain and L² is an unsubstituted methylene chain;
L¹ is an unsubstituted n-butylene chain and L² is an unsubstituted n-butylene chain;
L¹ is an unsubstituted methylene chain and L² is an unsubstituted n-butylene chain; or
L¹ is an unsubstituted methylene chain and L² is an unsubstituted methylene chain.

111. The compound of claim 108, having the formula:

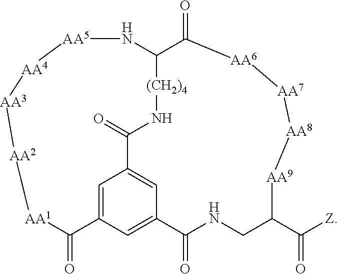

112. The compound of claim 104, wherein the compound of Formula 1 has the Formula 1-B: 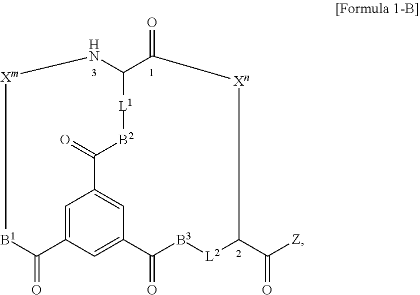
wherein Xⁿ has the formula: 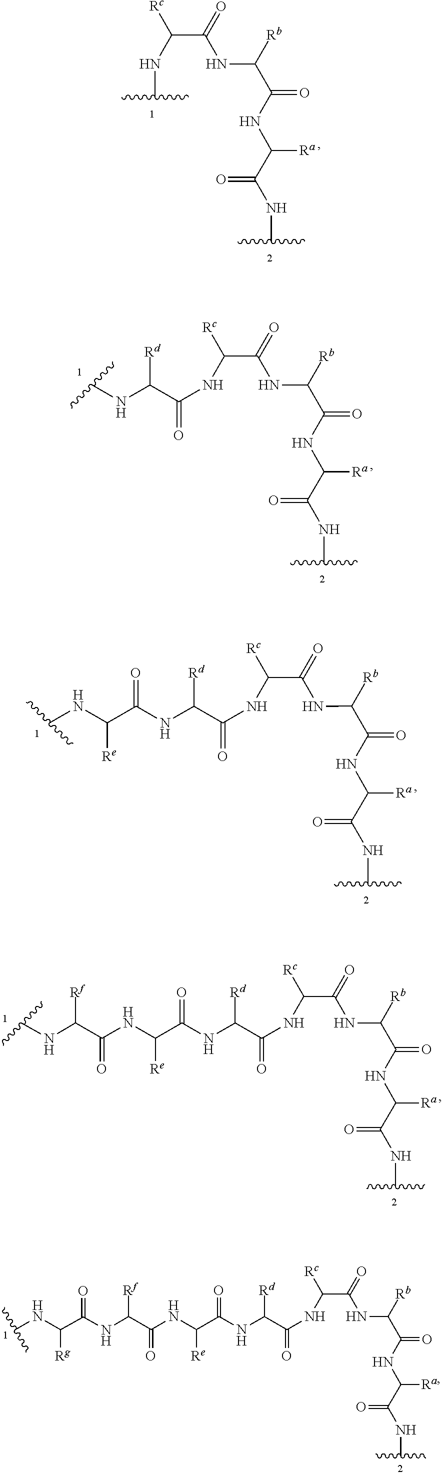
wherein wavy line 1 indicates the point of attachment to carbon 1 in Formula 1-B, wavy line 2 indicates the point of attachment to carbon 2 in Formula 1-B,
R^{a} has the formula: 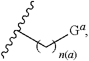
wherein n(a) is from 0-6, and G^{a} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, or CH(OH)CH₃.
R_{b} has the formula: 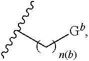
wherein n(b) is from 0-6, and G^{b} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{c} has the formula: 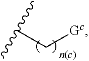
wherein n(c) is from 0-6, and G^{c} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{d} has the formula: 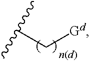
wherein n(d) is from 0-6, and G^{d} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{e} has the formula: 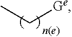
wherein n(e) is from 0-6, and G^{e} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{f} has the formula: 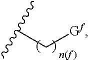
wherein n(b) is from 0-6, and G^{f} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{g} has the formula: 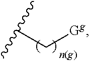
wherein n(g) is from 0-6, and G^{g} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃;
X^{m} has the formula: 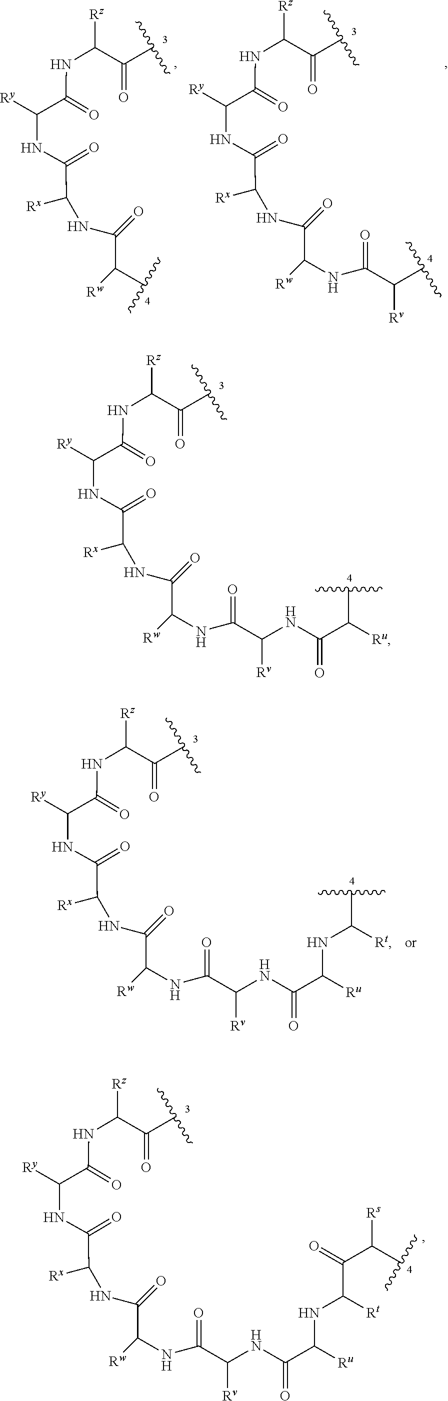
wherein wavy line 3 indicates the point of attachment to nitrogen 3 in Formula 1-b, wavy line 4 indicates the point of attachment to B¹,
R^{z} has the formula: 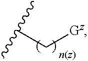
wherein n(z) is from 0-6, and G^{z} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{y} has the formula: 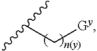
wherein n(y) is from 0-6, and G^{y} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{x} has the formula: 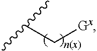
wherein n(x) is from 0-6, and G^{x} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{w} has the formula: 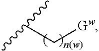
wherein n(w) is from 0-6, and G^{w} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{v} has the formula: 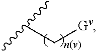
wherein n(v) is from 0-6, and G^{v} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{u} has the formula: 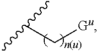
wherein n(u) is from 0-6, and G^{u} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{t} has the formula: 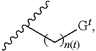
wherein n(t) is from 0-6, and G^{t} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.
R^{s} has the formula: 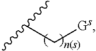
wherein n(s) is from 0-6, and G^{s} is hydrogen, C₁₋₆alkyl, C₃₋₈cycloalkyl, C₆₋₁₀ aryl, C₁₋₁₀ heteroaryl, OH, SH, SCH₃, COOH, CONH₂, NH₂, NHC(═NH)NH₂, COOH, CONH₂, or CH(OH)CH₃.

113. The compound of claim 112, wherein G^{a} is methyl, isopropyl, n-butyl, n-propyl, 3-methylprop-1-yl, 2-methylprop-1-yl, 1-napthyl, phenyl, 2-naphthyl, 3-naphthyl, or cyclohexyl, or G^{a} has the formula: 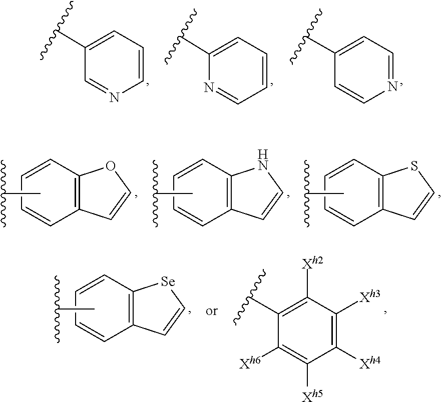
wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

114. The compound of claim 112, wherein n(a) is 1, 2, or 3, and G^{a} is CONH₂, NH₂, or NHC(═NH)NH₂.

115. The compound of claim 112, wherein G^{c} is methyl, isopropyl, n-butyl, n-propyl, 3-methylprop-1-yl, 2-methylprop-1-yl, 1-naphthyl, 2-naphthyl, 3-naphthyl, or cyclohexyl; or G^{c} has the formula: 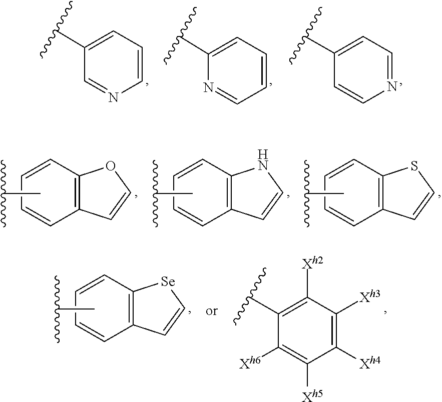
wherein:
X^{h2} is selected from H, OH, F, or Cl;
X^{h3} is selected from H, OH, F, or Cl;
X^{h4} is selected from H, OH, F, or Cl;
X^{h5} is selected from H, OH, F, or Cl;
X^{h6} is selected from H, OH, F, or Cl.

116. The compound of claim 112, wherein n(c) is 1, 2, or 3, and G^{c} is CONH₂, NH₂, NHC(═NH)NH₂.

117. The compound of claim 111, wherein X^{m} has the formula:

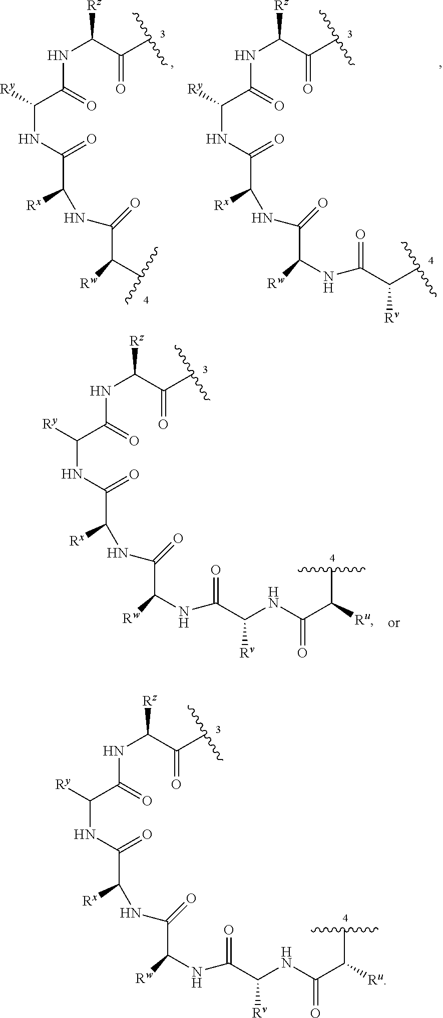

118. The compound of claim 112, wherein X^{m} has the formula:

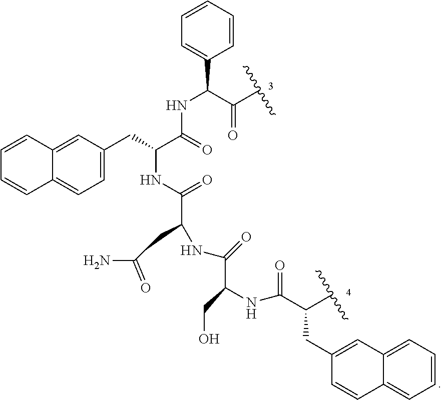 [SMILES: *C(=O)[C@@H](NC(=O)[C@@H](Cc1ccc2ccccc2c1)NC(=O)[C@H](CC(N)=O)NC(=O)[C@H](CO)NC(=O)[C@H](*)Cc1ccc2ccccc2c1)c1ccccc1]

119. The compound of claim 112, wherein Xⁿ has the formula:

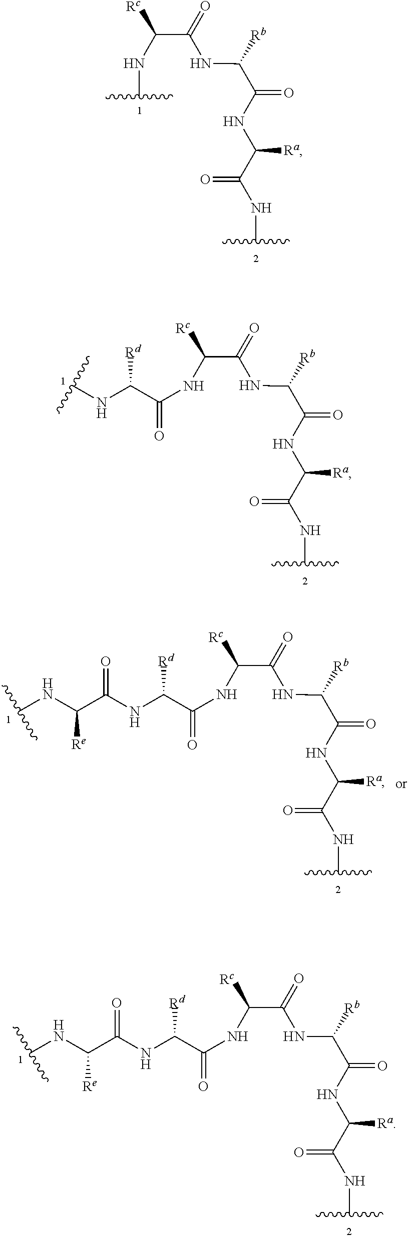

120. The compound of claim 112, wherein Xⁿ has the formula:

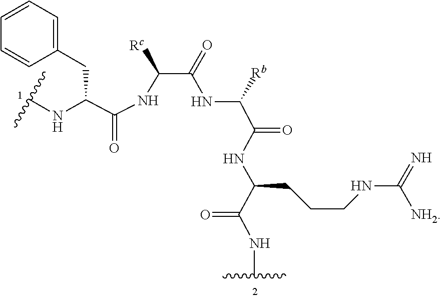

121. The compound of claim 112, having the formula:

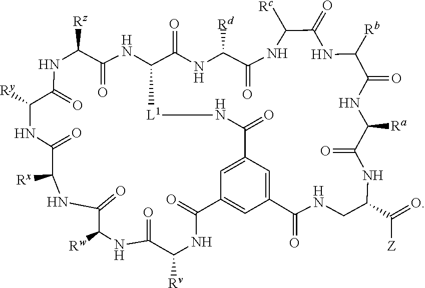

122. The compound of claim 112, having the formula:

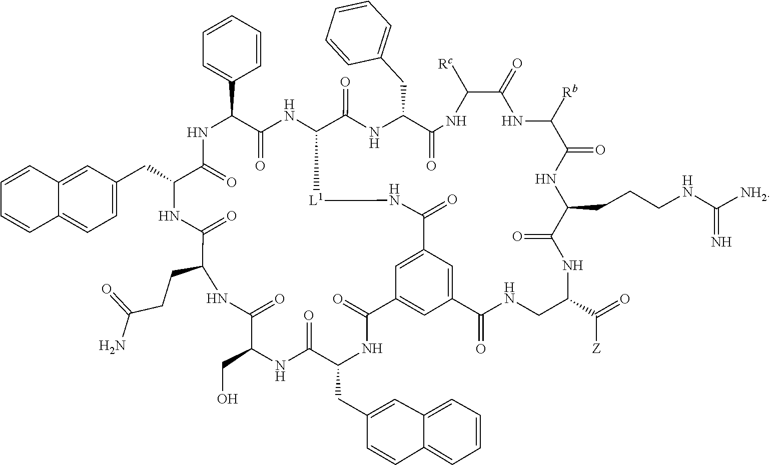

123. A method of treating cancer, comprising administering to a patient in need thereof a compound of claim 104.


SEQUENCE LISTING (3 sequences)

SEQ ID NO: 1 - protein, 8 aa, Artificial Sequence
  A synthetic sequence
  VARIANT (1)...(4): Xaa = a natural or non-natural amino acid
  SITE (1)...(4): Position may be absent
  VARIANT (5)...(7): Xaa = a natural or non-natural amino acid
  VARIANT 8: Xaa = absent or a natural or non-natural amino acid
  SITE 8: Position may be absent
XXXXXXXX

SEQ ID NO: 2 - protein, 5 aa, Artificial Sequence
  A synthetic sequence
  VARIANT 1: Xaa = L-ornithine, D-ornithine, L-lysine, or D-lysine
  SITE 2: Xaa = D-arginine
  VARIANT 3: Xaa = L-phenylalanine, D-phenylalanine, L-phenylglycine, or D-phenylglycine
  VARIANT 4: Xaa = from L-alanine, D-alanine, L-threonine, D-threonine, or glycine
  VARIANT 5: Xaa = a natural or non-natural amino acid
  SITE 5: Position may be absent
XRXXX

SEQ ID NO: 3 - protein, 4 aa, Artificial Sequence
  A synthetic sequence
  SITE 1: Xaa = L-ornithine
  SITE 2: Xaa = D-arginine
  SITE 4: Xaa = D-alanine
XRFA